(19) United States
(12) Patent Application Publication
(10) Pub. No.: US 2019/0294902 A1
(43) Pub. Date: Sep. 26, 2019

(54) METHOD AND APPARATUS FOR PROGRAMMABLE, REAL-TIME, MULTI-DOMENSIONAL OBJECT PATTERN RECOGNITION ALGORITHMS (OPRA)

(71) Applicant: Dario CROSETTO, DeSoto, TX (US)

(72) Inventor: Dario CROSETTO, DeSoto, TX (US)

(21) Appl. No.: 16/345,263

(22) PCT Filed: Oct. 30, 2017
(86) PCT No.: PCT/US2017/059133

§ 371 Date: Apr. 26, 2019

Related U.S. Application Data
(63) Continuation-in-part of application No. 15/338,256, filed on Oct. 28, 2016, now abandoned.

(51) Int. Cl. G06K 9/00; G16H 30/20; A61B 6/03; A61B 6/00

(52) U.S. Cl. CPC G06K 9/00986; G16H 30/20; G06K 2209/05; G06K 9/00214; A61B 6/5217; A61B 6/037

(57) ABSTRACT

The present 3D-Flow OPRA is a revolutionary electronic instrument for multiple applications: advancing science, saving lives, finding and tracking fast moving objects, etc. It allows to build a flexible, scalable, technology-independent, cost effective powerful tool to uncover the unknown and to confirm or exclude the existence of a subatomic particle predicted by theoretical physicists. When used for Medical Imaging applications the 3D-Flow OPRA allows to accurately measure minimum abnormal biological processes of diseases at an early curable stage such as cancer in a 3D-CBS (3-D Complete Body Screening), improving diagnosis and prognosis to maximize reduction of premature deaths and minimize cost per each life saved. Both instruments, the 3D-Flow OPRA and the 3DCBS can benefit from the additional ER/DSU invention also described in this non-provisional patent application, which allows to record real data from detectors.

### CROSS REFERENCE TO RELATED APPLICATIONS (IF ANY). (RELATED APPLICATIONS MAY BE LISTED ON AN APPLICATION DATA SHEET, EITHER INSTEAD OF OR TOGETHER WITH BEING LISTED IN THE SPECIFICATION.)

This application claim prior provisional application filed Oct. 28, 2016, Ser. No. 15/388,256 entitled “METHOD AND APPARATUS TO BUILD NEW ELECTRONIC INSTRUMENTS AND DEVICES: THE 3D-FLOW OPRA TO SOLVE APPLICATIONS OF FAST, REAL-TIME, MULTI-DIMENSION OBJECT PATTERN RECOGNITION ALGORITHMS (OPRA) AND THE 3D-CBS (3-D COMPLETE BODY SCREENING) TO ACCURATELY MEASURE MINIMUM ABNORMAL BIOLOGICAL PROCESSES OF DISEASES AT AN EARLY CURABLE STAGE SUCH AS CANCER IMPROVING DIAGNOSIS AND PROGNOSES FOR MAXIMIZING REDUCTION OF PREMATURE DEATHS AND MINIMIZE THE COST PER EACH LIFE SAVED”, the disclosure of which is incorporated herein in its entirety by reference thereto.

### STATEMENT OF FEDERALLY SPONSORED RESEARCH OR DEVELOPMENT (IF ANY)

No Federally sponsored research for this application.

### THE NAMES OF THE PARTIES TO A JOINT RESEARCH AGREEMENT IF THE CLAIMED INVENTION WAS MADE AS A RESULT OF ACTIVITIES WITHIN THE SCOPE OF A JOINT RESEARCH AGREEMENT

This claimed invention was not made as a result of activities within the scope of a joint research agreement, it is solely the invention of the author Dario B. Crosetto.

### REFERENCE TO A “SEQUENCE LISTING,” A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC. THE TOTAL NUMBER OF COMPACT DISC INCLUDING DUPLICATES AND THE FILES ON EACH COMPACT DISC SHALL BE SPECIFIED

No “Sequential Listing”, table, or computer listing appendix on a compact discs is submitted with this application.

### BACKGROUND OF THE INVENTION

The innovative 3D-Flow parallel-processing architecture, and fault-tolerant system, recognized valuable by major, public, scientific reviews, proven feasible and functional in hardware, breaks the speed barrier in real-time applications such as in High Energy Physics (HEP) and Medical Imaging. It is flexible, scalable, programmable, modular, and technology-independent since it is able to migrate to the most advanced and cost-effective technology.

It creates a revolution in the field because it provides experimenters with a new instrument to test the efficacy of their theory and apparatus just as an oscilloscope or a Logic State Analyzer tests the theory of a circuit design and that its implementation is working properly.

The 3D-Flow instrumentation for fast, real-time Object Pattern Recognition (OPRA) on data arriving in parallel from thousands of sensors at a very high speed with its accessories such as LHC TER/DSU, 3D-CBS/DSU, RAU, ATCA-PRAI, etc. is like an Oscilloscope or Logic State Analyzer with their accessories such as a Trimode differential probe, a DDR3 SODIMM Interposer, etc.

To understand the difference between the new 3D-Flow OPRA, an oscilloscope and a Logic State Analyzer, instrumentations existing for many years, I provide here a short description of each one—what is it and what it is used for.

What is an oscilloscope? It is a type of electronic instrument that allows observation of constantly varying signal voltages, usually as a two-dimensional plot of one or more signals as a function of time.

Modern digital instruments may calculate and display these properties directly. However, oscilloscopes are somewhat limited with only two or four input channels to correlate a small number of digital, analog and serial signals.

Which applications can benefit from an oscilloscope? It is used to visualize and measure the characteristic of signals and to troubleshoot malfunctioning equipment. The usefulness of an oscilloscope is not limited to the world of electronics. With the proper transducer, an oscilloscope can measure all kinds of phenomena. A transducer is a device that creates an electrical signal in response to physical stimuli, such as sound (microphone), mechanical stress, pressure, light, or heat.

What is a Logic State Analyzer? A logic analyzer is an electronic instrument that captures and displays multiple signals from a digital system or digital circuit. It may convert the captured data into timing diagrams, protocol decodes, state machine traces, assembly language, or may correlate assembly with source-level software. Logic Analyzers have advanced triggering capabilities, and are useful when a user needs to see the timing relationships between many signals in a digital system. A logic analyzer can be triggered on a complicated sequence of digital events, then capture a large amount of digital data from the system under test.

Which applications can benefit from a Logic State Analyzer? Logic analyzers provide an ideal tool to verify and debug complex designs for electrical engineers. Logic analyzers are useful when multiple signals must be observed simultaneously, as well as when you need to look at a system's signals in the same way its hardware does. The biggest difference from the oscilloscope is the extra input channels it offers.

The implementation of the verifiable 3D-Flow system with the capability of extracting ALL valuable information from radiation, which can save taxpayers billions of dollars by providing a very powerful tool to discover new particles and benefit humanity with an effective early cancer detection potentially saving millions of lives. Unlike the traditional Level-1 Trigger algorithm implementation in “cabled-logic” which provides the capability to execute one algorithm, or the CERN-CMS approach of executing 128 algorithms, or the costly lower performance FPGA approach, the 3D-Flow architecture allows the execution of trillions of different algorithms like a generic processor, but has the advantage over any Pentium, ARM, SPARC, Hypercube, etc., processor/architecture in that it can execute specialized instructions (or “OPRA steps” for an optimized Object Pattern Recognition Algorithm) to identify particles with the capability to execute at each “step” up to 26 operations such as add, subtract, compare with 24 values, etc., in less than 3 nanoseconds. The 3D-Flow performance is further increased by its bypass switch and NEWS communication channels with neighbors.

Certainty of results can be obtained by accurately measuring a few controllable variables, but funding innovations that can accurately measure these variables is not provided and the dissemination of these technological breakthrough are even boycotted, oppressed, suppressed.

We have known for over 60 years (confirmed by experimental results) that when cancer is detected at an early stage it can be successfully cured and save 5% of all cancer deaths. However, we do not have effective drugs to cure the majority of late stage cancers (these very expensive drugs on average prolong the patient's life for a few months when cancer is detected at an advanced stage).

What is needed to save many lives, therefore, is an effective early cancer detection because we have already the means (surgery, radiation therapy, drugs, etc.) to cure cancer if it is detected at an early stage.

The logical investigation is to look at the best signals provided by the mutation of the very first normal cells into cancerous cells and accurately detect those signals to obtain an effective early detection.

Among changes in odor, temperature, tissue conductivity, density, fluorescence, etc., caused by the start of the development of cancer, the most reliable is a change in metabolism because cancer cells are growing faster than normal cells and consume from 5 to 70 times more nutrient than cancer cells. Associating a radioisotope emitting 511 keV photons in opposite directions to a molecule of nutrient (Oxygen-15, Ammonia, C-11, Glucose FDG, Rubidium-82, etc.) and accurately measuring the impact point of the photons in the detector (particle detection), gives us the possibility to track the minimum abnormal consumption of nutrient from body cells in different organs and parts of the body. Measuring a few of these variables (changes in tissue density, fluorescence, etc.) can further improve the certainty of results, and for this reason we have multimodality instrumentations (PET/CT, PET/MRI, PET/Ultrasound, etc.), blood tests and ultimately the biopsy to look at the cells' structures through a microscope.

Because the most important variables that need to be measured in a non-invasive test that can be extended to a large population at a low radiation dose and low examination cost depend on accurately extracting and measuring all the relevant information for specific modality (511 keV for PET, 140 keV for SPECT, 60 keV for CT, etc.) from the many signals generated from radiation, it is necessary to address improvements and techniques in particle detection.

The measurements of the few characteristics (or variables) of these photons are controllable. They are: photon's total energy, arrival time, coordinates of the impact point in the detector, high signal-to-noise ratio and the capability to capture as many signals as possible at a very high input data rate and filtering them from the many other signals from radiation.

Inventions (the 3D-CBS, “3-D Complete Body Screening” and the MR/3D-CBS) and new techniques can improve measurements of each of the above variables. Calculations and logical reasoning can predict the advantages from each new technique. I trust that a scientist will recognize and explain research in physics where the variables are limited, and calculations and logical reasoning can predict with high accuracy the expected results of an invention designed to solve technical problems. Whereas in the field of medicine, because our body is very complex, what was stated at the end of the movie that “the true nature of a body cell no one fully understood” is true, as the variables in the body are thousands or even millions, difficult if not impossible to control; this allows scientists to make only a “guess” rather than estimate results with any accuracy.

A. Background Material Relative to these Inventions Related to the Project's Research Plan

The challenge in High Energy Physics experiments is to identify good events at the lowest cost per each good event captured from data relative to over a billion collisions per second arriving from the detector at a rate higher than 40 million events per second.

As analogy, it would be like watching a movie from a projector showing high resolution frames, each containing billions of bacteria (or a live event) sped up to 40, 80, or over 100 million frames per second instead of the usual 24 frames per second, and trying to identify and extract all frames containing a rare bacteria with a specific shape occurring on average once every 10 billion frames, without being able to slow the movie down or store the data for later examination.

Typically a few micrometers in length, bacteria have a number of shapes, ranging from spheres to rods and spirals.

The 3D-Flow system is basically a “decision box” or “Trigger” having the task to process accurately and at great speed each high resolution image and decide whether to keep it or trash it without the possibility to store all of them because in one day the data would fill all the hard drives on the planet.

Finding the rare Higgs boson-like particle or any new particle among billions generated each fraction of a second that experimenters would like to find when the data received satisfy their Object Pattern Real-Time Recognition Algorithm is like finding a rare bacterium among billions of bacteria shown at very high speed.

In Medical Imaging application the challenge is to extract all valuable information from radiation (emitting hundred millions signals per second) related to tumor markers (or other biological processes) in order to reduce the radiation dosage to the patient and identify the smallest irregular biological process at the lowest cost per valid signal captured from these tumor markers. This would provide an effective early cancer detection (or detection of anomalies in other biological processes) at a very low radiation dosage and at an affordable examination cost.

The challenge for both applications is to design a “decision box” called Trigger that can analyze each frame in real-time and can find the rare object or new particle in physics (or anomalous biological process in medicine).

Crosetto's basic invention is the 3D-Flow architecture capable of executing experimenters' desired programmable complex Object Pattern Real-Tune Recognition Algorithms (OPRA), while sustaining an input data rate of over 80 million events per second from over a billion collisions per second, with zero dead-time, and a lower cost per each event captured than other approaches.

For Medical Imaging applications, his basic 3D-Flow invention is combined with other inventions he conceived after the year 2000. They allow all valuable information to be extracted from the tumor markers (linked to radiation) at the lowest possible cost.

Crosetto has explained his basic invention in one page. (See FIGS. 9, 10 and 11) and used a practical analogy to explain his inventive concept to middle school students in a book [36] and to high school students in a video &&].

Besides contributing to the creation of new powerful tools for the discovery of new particles, Crosetto's goal is to reduce cancer deaths and cost.

One way to increase accountability and achieve this goal would be to demand that
1. funding agencies which use taxpayer money to fund projects request a plan to demonstrate the efficacy of the proposed project that should be tested on a test-bench like is submitting this proposal in FIG. 38, FIG. 39, FIG. 40, FIG. 41 and FIG. 42, where the “Hardware Detector Simulator” on the left of the figure send at the desired speed from 1 to 80 MHz 8,192, 16-bit words to the proposed system and the “Results Analyzer Unit” on the right of the figure verifies if ALL “rare” events embedded in the most difficult noisy environment of pileup, and ambiguous pattern to be recognized are found in real-time by the proposed project
2. funding agencies which use taxpayer and donation money to fight cancer, whether through a new drug, vaccine, medical imaging device, or healthy lifestyle promotion, etc., estimate the reduction of cancer deaths and cost they expect to attain with their project (or combined with other existing techniques) and present a plan to test it on a sample population. For example, test the plan on 10,000 people ages 55-74 taken from a location where the mortality rate has been constant for the past 20 years. A difference or no difference in mortality rate will quantify the success or failure of the proposed solution. (See FIG. 68).

1. Benefits of PI's Inventions in the Fields of HEP Particle Detection and Medical Imaging with a Single Modular Industrialized Board with 68×3D-Flow Processors.

### The 3D-Flow DAQ IBM PC Board FIG. 14 for Particle Detection and Photon Detection in PET and PET/CT

In December 2002 Crosetto designed and in March 2003 built and tested in hardware a modular board FIG. 14 system that would guarantee a difference between any two clock signals among hundreds of thousands of channels of less than 40 picoseconds.

The article title “3D-Flow DAQ IBM PC board for Photon Detection in PET and PET/CT” was presented at the IEEE-NSS-MIC Conference in 2003 and is available at the Conference Record. M3-130. http://www.crosettofoundation.com/uploads/105.pdf

A study of the scientific literature before and after my invention shows that the effort to develop fast, expensive, high power consumption Ga-As. ECL, etc., circuits for level-1 trigger has become obsolete because my invention can use cost-effective technology, sustain a high input data rate, while at the same time executing complex real-time uninterruptable algorithms for a time period longer than the interval between two consecutive input data. However, as occurs with many breakthroughs, it takes time to fully uncover and develop all the benefits that I integrated with other inventions in detector assembly, segmentation, and coupling of detectors to the electronics that go beyond the original 3D-Flow parallel-processing architecture. The basic 3D-Flow invention, together with other inventions I developed after the year 2000 it can greatly benefit medical imaging applications, thus making effective early cancer detection possible while lowering radiation exposure and the examination cost.

The role of Positron Emission Technology (PET) should be changed with use of the 3D-CBS (Three Dimensional Complete Body Screening) for maximizing the capture of signals that will detect minimum abnormal metabolism (or other biological processes), achievable by capturing simultaneously and accurately as many signals as possible from the tumor markers from all organs of the body in order to identify the smallest anomaly, at the lowest cost per signal captured and requiring the minimum radiation to the patient. This paper provides scientific arguments for setting new parameters for industry to establish the correct relation between the goal of obtaining substantial reduction in cancer deaths and the implementation of innovations and technology that will provide the expected results through early cancer detection

The “Cancer Research Projects Comparison Table”, reporting so far 124,737 cancer research projects already funded for a total of $37 Billion, translates into practice the above points 1 and 2 and when “implemented consistently” becomes the tool that could lead to a substantial reduction of premature cancer deaths and cost for each life saved. (www.crosettofoundation.org/table.php?lang=en). The projects with the highest potential to reduce cancer deaths and the ones that are a waste of money will stand out first using the powerful tools of the table and then through the dialogue among their Principal Investigators who would be required to support their claims with scientific arguments.

For the first objective the table allows searching and sorting data on: 1. Projects that provide the highest estimate, supported by scientific arguments, of cancer deaths, 2. Active projects that have received conspicuous funding without providing an estimate of results as far as cancer death reduction, 3. Projects ended without providing results in reduction of cancer deaths, and other information.

For the second objective, since it is impossible to set up a live discussion among all 124,000 projects, it makes sense to compare any project under consideration with one which received awards, high funding or which claims (supported by solid scientific arguments) the highest reduction in cancer deaths. A practical example of an ongoing Public, Open “Scientific Procedure” is the one being implemented for some time, which involves emails to experts, workshops at CERN, meetings at BNL, as well as at the University and Polyclinic S. Matteo of Pavia to make the scientific truth prevail on projects with highest potential to reduce cancer deaths and cost [46] (www.crosettofoundation.org/uploads/408.pdf. After several meetings broadcast worldwide over several years on the subject of evaluating in depth the innovative 3D-CBS technology for early cancer detection by the author Dario Crosetto, on Oct. 28, 2010 a worldwide meeting (connected via EVO Caltec system to U.S., Canada, CERN, etc.) was organized at the University of Pavia (Italy) to compare the 3D-CBS project that has been waiting for funding for more than a decade with the Axial-PET project by Christian Joram that won the first prize at the Workshop “Physics for Health” at CERN on Feb. 3, 2010. Surprising and shocking facts are emerging from this scientific procedure, some of which are summarized with testimonials in the YouTube video available at: http://www.youtube.com/watch?v=65M15 ddlvU. This scientific procedure is core to the implementation of the comparison table and should not be limited to these two projects but by using this table, any cancer research project that can claim higher results in reducing cancer deaths and costs can be compared with those that received awards.

### BRIEF SUMMARY OF THE INVENTION

What is the 3D-Flow OPRA? The 3D-Flow OPRA is a new electronic instrument and device to solve target application problems of fast, real-time multi-dimension Object Pattern Recognition (OPRA) on data arriving in parallel from a matrix of thousands of sensors (or transducers) at a very high speed that are sent to an equivalent matrix of thousands of 3D-Flow processors.

The 3D-Flow architecture provides data exchange (2×2, 3×3, 4×4, 5×5, . . . ) with neighboring processors, while its bypass switch allows the execution of uninterruptable algorithms for a time longer than the interval between two consecutive input data sets. Each 3D-Flow processor can execute several different programmable “OPRA steps,” called OPRAS, each consisting of up to 26 operations such as adding, subtracting, comparing to 24 values, etc., in less than 3 nanoseconds.

The result is that it can execute users' desired programmable complex Object Pattern Real-Time Recognition Algorithms (OPRA) comparing the desired object (shape and detailed characteristics) with billions of objects per second, while sustaining an input data rate of several million frames per seconds, with zero dead-time.

It can measure all kinds of phenomena and identify all kinds of objects in 3-D that create an electrical signal in response to physical stimuli.

For example, a shape of different colors, a shape of different levels of heat, a shape of different levels of sound volume and frequencies, a shape of different energies, a shape of different mechanical stress, a shape of different pressure, a shape of different light, the characteristics of a specific subatomic particle measured from signals generated by CCD, APD, PMT, SiPM, PADs, silicon strip detectors, wire-chambers, drift-chambers, etc.

It can find an object that can match several of these properties combined. For example:
a) by combining on the same channel to the 3D-Flow processor array the information from a movie camera in the visible light with another in the infrared light, with a radar, a lidar, etc. Using thousands of movie cameras, each covering a tiny field of view and sending the images to a 3D-Flow OPRA system in a VME crate with up to 5,120 channels or to a VXI 3D-Flow OPRA crate with up to 10,240 channels. For example, each channel carries the information of 8-14 bit color, 8-14 bit heat (infrared light), other bits for the radar and Lidar information, etc.
b) by combining on the same channel to the 3D-Flow processor array the information from PMT, SiPM, Pad transducers on the calorimeter Trigger Tower 108, from a wire chamber, drift chamber, silicon strip detector, etc. in the same view angle. Using a detector with 2,304 detector arrays in a PET/3D-CBS device or 8,192 Trigger Towers 108 in HEP applications. For example, each channel carries 8-bit energy information, a few bits of time information and spatial resolution, etc.

Data from a matrix of different types of sensors or transducers are transferred to an equivalent matrix of 3D-Flow processors at the maximum rate of 1.28 Gbps per channel. The maximum transfer rate for a 5,120 channel VME crate 3D-Flow OPRA system is 6.5 Tbps and for a 10,240 channels VXI 3D-Flow OPRA system is 13.1 Tbps.

The 3D-Flow OPRA has advanced triggering capabilities, and are useful when a user needs to find a specific object or multiple objects with a specific timing relationship between them, or needs to see the timing relationship between several objects in a digital system. It can trigger on a specific multidimensional object which has been identified by measuring different kinds of phenomena that create an electrical signal. It can trigger on a complicated sequence of digital events.

When trigger conditions are met, it can save the data of the event(s) with the rare object(s) and time-stamp for subsequent visualization by the user.

It can correlate and trigger on two identified objects located far apart in the array but within a specific relation in time; for example, it can identify back-to-back photons in the annihilation of a positron with an electron in the 3D-CBS Medical Imaging application.

Which Applications can Benefit from the 3D-Flow OPRA?

In HEP, this 3D-Flow OPRA system or instrumentation can be used for Level-1 Trigger to extract all valuable information from radiation to capture the new desired particle theorized by physicists.

In Medical Imaging, this 3D-Flow OPRA system or instrumentation can be used to extract all valuable information from radiation to enable the detection of cancer and many other diseases at an early curable stage, as well as allowing a significant reduction in the radiation dose given to the patient. Its effectiveness will save many lives [¹] and the low examination cost will reduce healthcare costs.

In a multi-lens movie camera application, the 3D-Flow OPRA system or instrumentation can be used to recognize objects from data arriving from thousands of movie cameras each looking at tiny details with a very small field of view.

And it can benefit many other real-time applications.

Features & Implementation Types and Costs

Because the main objective is fast object pattern recognition on data arriving at a very high speed which needs to be exchanged between neighboring processors, one of the most important requirements is to keep the length of the cables between adjacent 3D-Flow processor boards as short as possible and the entire system in a small compact box. Neighboring processors in different chips or boards will require the longest time to transfer data (e.g. a 30 mm wire requires more than 100 picoseconds).

Although the 3D-Flow architecture that I invented in 1992 can satisfy experimenters' requirements to execute programmable complex algorithms at the highest LHC bunch-crossing rate with zero dead-time, using longer cables will increase the algorithm's execution time and the number of 3D-Flow processor layers; this in turn will increase the power consumption of the system, making the system bigger as it needs to dissipate more power which will further increase the cable length in a cycle that will increase the cost of the system until an optimized geometry balances all these parameters. (The number of layers of 3D-Flow processors needed is calculated by dividing the time needed to execute an algorithm by the time interval between two consecutive input data and rounding the result up to the next integer). This explains why keeping the cable length between adjacent processors in different chips, boards and crates as short as possible and keeping the system as compact as possible will maximize performance at the lowest cost.

In 1994, I proposed the implementation of a 1280 channel 3D-Flow system in a cylindrical geometry 1 m in diameter×1.8 m tall (reflecting the cylindrical geometry of the detector: calorimeter in HEP and PET in Medical Imaging) with the longest cable between adjacent 3D-Flow processors boards only 13 cm. The same cylindrical geometry remains today the most cost-effective solution to achieve highest performance at the lowest cost, and with today's technology the cylindrical dimensions for an 8,192 channel 3D-Flow system for Level-1 Trigger of a large experiment at LHC would be 40 cm in diameter and 80 cm tall with the longest cable between 3D-Flow processor boards only 8 cm.

However, for practicality, one can give up some cost-efficiency and use the most convenient form factor which can be VXI, VME, ATCA, Micro-ATCA, VPX, etc.

In this proposal, FIG. 2, FIG. 3, FIG. 4, I have selected VXI 490 495 for the large boards and in FIG. 5, VME 300 for the smaller boards; however, if there is a specific requirement from CERN or another user, they can be implemented in any form factor because the backplane is custom designed. Among the considerations made in selecting VXI and VME was the fact that they are more economical per volume of electronic circuits implemented, that there are already many of these crates at CERN that can be reused from dismissed electronics, and that the 43,008×3D-Flow processors for a 8,192 channel 3D-Flow system fit into a compact VXI volume 36 cm×36 cm×24 cm, and 25,600×3D-Flow processors for 4,096 channels fit into a compact VME cube 16 cm×16 cm×16 cm minimizing the distance to exchange data between neighboring processors in different chips and boards.

After proving feasibility and functionality of the 3D-Flow invention in hardware in two modular boards each with 68×3D-flow processors implemented in large FPGAs, the major components to build a 3D-Flow system have been quoted by several companies with a reputable record of working products (some have a catalogue of products they build and commercialize). Some components of the 3D-Flow system have been quoted by two or three companies in competition showing feasibility.

The following report is a comparison between the cost of this new instrument—the 3D-Flow OPRA system with some of its accessories and the price of Oscilloscopes and Logic State Analyzers with some of their accessories. Note that the price per channel of the mainframe unit and cables or probes of the accessories of the 3D-Flow OPRA system is very competitive with the price of the mainframe and probes of the oscilloscopes and Logic State Analyzers.

The budgetary quote of the mainframe 3D-Flow system in a VXI and VME form factor and its accessories reported below when ordered with this R&D proposal is based on the estimates received in the quotes provided in the budget justification of this proposal. When these products become available by a manufacturing company that includes them in their catalogue, the price will be determined by the manufacturing company according to the market value.

TABLE 1
Price comparison for Mainframe Instrumentation

| | | Cost per |
|---|---|---|
| Item | Unit cost | channel |
| 4 ch. Oscilloscope 33 GHz (Tektronix DPS77004SX) | $479,000 | S119,750 |
| 4 ch. Oscilloscope 23 GHz (Tektronix MSO72304DX) | $203,000 | S50,750 |
| 4 ch. Oscilloscope 4 GHz (Tektronix MS0070404C) | $44,900 | S11,225 |
| 136 ch., Logic State Analyzer (Tektronix TLA7BB4 + TLA7012) | $99,600 | $732 |
| 2,304 ch., 3D-Flow OPRA System, VME version with 14,400 × 3D-Flow | $46,500$ | $20 |
| processors for 3D-CBS Medical Imaging for detection of cancer and many | | |
| other diseases at an early curable stage, for accurate diagnoses, prognoses | | |
| and efficiently monitoring of treatments. (Crosetto - Patent Pending - | | |
| 2304ch-20MHz-64bit-120-OPRAS) | | |
| 4,096 ch., 3D-Flow OPRA System, VME version with 26,500 × 3D-Flow | $78,500 | $19 |
| processors for a programmable, zero dead-time Level-1 trigger for LHC | | |
| experiments. (Crosetto - Patent Pending - 4096ch-80MHz-16bit-30- | | |
| OPRAS) | | |
| 8,192 ch., 3D-Flow OPRA System, VXI 490 version with 43,008 × 3D- | $98,000 | $12 |
| Flow processors (see FIG. 69, FIG. 70, FIG. 71 for a programmable, zero | | |
| dead-time Level-1 Trigger for LHC experiments for the discovery of new | | |
| particles. (Crosetto - Patent Pending - 8192ch-80MHz-16bit-20-OPRAS) | | |

TABLE 2
Price Comparison for Probes

| | | Cost per |
|---|---|---|
| Item | Unit cost | channel |
| 4 Oscilloscope Probe - 23 GHz - 4 × 23 GHz TriMode Differential Probe | $86,000 | $21,500 |
| (Tektronix P7520A). | | |
| 4 Oscilloscope probe - 4 GHz - 4 × 4 GHz Probe (Tektronix P7504) | $23,600 | $5,900 |
| 136 ch. Logic State Analyzer Probe 4 × 34 Probes (Tektronix P6910) | $37,600 | $276 |
| 2,304 ch. 3D-Flow OPRA Probe - 18 × 128 × 10 Gbps per Twinax 0.5 m | $2,700 | $1.17 |
| cable and 2 × 400-pin connectors (Crosetto - Patent Pending) | | |
| 4,096 ch. 3D-Flow OPRA Probe - 32 × 128 × 10 Gbps per Twinax 0.5 m | $4,800 | $1.17 |
| cable and 2 × 400-pin connectors (Crosetto - Patent Pending) | | |
| 8,192 ch. 3D-Flow OPRA Probe 10 Gbps per Twinax 1 m cable and 2 × | $10,880 | $1.32 |
| 400-pin connectors (Crosetto - Patent Pending) | | |

TABLE 3
Price Comparison for Pattern Generators

| | | Cost per |
|---|---|---|
| Item | Unit cost | channel |
| 2 ch. Arbitrary Waveform Generator 1.2 Gsps, 14-bit per channel | $47,300 | $23,650 |
| (Tektronix AWG5012C) | | |
| 64 ch. Pattern Generator 300 MHz (Tektronix PG3A ) | $22,600 | $353.12 |
| 181 ch. DDR3 SODIMM interposer (Nexus Technologies NEX- | $57,700 | $318.78 |
| SODDR3INTR-XL) | | |
| 2,304 ch. Pattern Generator & Event Recorder @ 320 MHz (640 Mbps) | $23,000 | $9.98 |
| per channel, 3D-CBS ER/DSU for testing the 3D-Flow OPRA for the 3D- | | |
| CBS Medical Imaging applications. (Crosetto - Patent Pending) | | |
| 8,192 ch. Pattern Generator & Event Recorder @ 320 MHz (640 Mbps) | $40,000 | $4.88 |
| per channel, LHC TER/DSU for testing the 3D-Flow OPRA for a | | |
| programmable, zero dead-time Level-1 Trigger for LHC experiments. | | |
| (Crosetto - Patent Pending) | | |
| 4,096 ch. Pattern Generator & Event Recorder @ 640 MHz (1280 Mbps) | $60,000 | $14.64 |
| per channel, LHC TER/DSU fortesting the 3D-Flow OPRA for a | | |
| programmable, zero dead-time Level-1 Trigger for LHC experiments. | | |
| (Crosetto - Patent Pending) | | |

B. Verifiable Invention: This Proposal Provides a Complete Set of Tools Able to Verify that the 3D-Flow Invention Delivers What it Promises: The Capability of Discovering New Particles, and a Reduce Cancer Deaths and Costs when Tested on a Sample Population

FIG. 12, FIG. 34 and FIG. 38 illustrates the invention process flow from concept, to simulation, to the design of details, to the verification in hardware of the different parts, to the testing on a sample population.

VERIFICATION: The 3D-Flow System has been proven feasible and functional in hardware in two modular boards. Over ten companies with a record of products showing competence have provided quotes to build all parts showing feasibility. Efficacy of the 3D-Flow system in extracting ALL information from radiation can be verified on a test bench with the DSU.

FINAL VERIFICATION IN SAVING LIVES AND REDUCING HEALTH CARE COSTS: Fund the NRE, fund the 3D-CBS device, test it on 10,000 people ages 55-74 taken from a location where the mortality rate has been constant for the past 20 years.

The expectation is that it will reduce cancer deaths by 33% in 6 years and 50% in ten years and cut at least in half the cost per each life saved, thus significantly reducing healthcare costs. Why an invention shown feasible and beneficial has not been funded?

A. The 3D-Flow Invention Advances Science by Providing a Very Powerful Tool for Discovering New Particles

The proposed implementation of the 3D-Flow system is verifiable for any implementation selected. The left section of FIG. 39, FIG. 41 and FIG. 42 shows the Detector Simulator Unit (DSU) 200 accommodating 16×DSU boards 210 that could replace the billion dollar LHC experimental setup with a unit costing approximately $40,000 generating 15 million (or 60 million) events (each with a size 8,000 channels×16-bit) stored in 256 GB (or 1 TB) RAM memory sending them out at the current 40 MHz LHC bunch-crossing rate with the possibility to double this rate to verify that this proposed 3D-Flow system implemented in one crate will also work ten years plus from now when the LHC will a much higher luminosity and it will be necessary to acquire 32-bit from each of the 8,192 trigger channels carrying information from additional sub-detectors.

2. A 1.3 TB/Sec 3D-Flow System for 8,192 Channels Implemented in One VXI Crate Having the Capability to Execute Up to 20 OPRA Steps/Event/Channel @80 MHz, 16-Bit/Channel

The left column of FIG. 39 shows the LHC TER-Trigger Event-Recording and Simulator 200 (generically known as: DSU—Detector Simulator Unit):
This would provide the opportunity for any university or research center to have on the test bench of their laboratory a box of electronics costing approximately $40,000 providing the exact functionality of a multi-billion dollar LHC detector and collider except that it would be in their lab generating up to 16-bit/channel events at the current LHC 40 MHz bunch-crossing rate, or 32/bit/channel events from future experiments, and a rough environment of very high luminosity planned to be available 10 to 15 years from now.
   This way, “remote” experimenters at their home university will not only be able to test the current Level-1 Trigger with real raw trigger data and prepare a working system for 10 to 15 years from now, but they will also be able to manually edit any raw data of the 15-120 million events stored in the memory of the DSU 210, OR 220, creating the most difficult patterns, with pileup events and other very noisy conditions to make it more difficult for the 3D-Flow System to extract all valuable information from the most unexpected environments. This would thoroughly test the performance and capability of the 3D-Flow System to analyze up to 32-bit/data per channel carrying the information from billions of collisions per second arriving in parallel from 8,192 channels at 40 million events/sec and filtering the good events.
This would provide the opportunity for CERN and other laboratories with particle accelerators and detectors that run million-dollar or billion-dollar experiments, to record for 0.2 seconds 256 GB of real trigger row data (or for 1.6 seconds, recording 2 TB of data, if the 128×2 GB SODIMM memory modules are replaced with 128×16 GB SODIMM memory modules) from all the detectors (calorimeter, tracking, etc.) FIG. 2 connected through the PRAI boards 130, 140 (Patch Panel Regrouping Associates Ideas) to the DSU crate. The 256 GB (or 2 TB) raw-data file can then be sent to the Scientific Associates at universities in different parts of the world who are participating in the experiments and have designed parts of the electronics. This 256 GB (or 2 T) of raw-data recorded at CERN or other accelerator sites by a TER/DSU unit (Trigger Event Recorder, the same DSU unit working in acquisition mode rather than generating signals to be sent to the 3D-Flow System in Simulator mode) can be loaded into the DSU System on the test bench at the remote laboratory of a university participating to the experiment. This precious data will provide the Scientific Associates at the remote university the means to not only test the functionality and performance of the 3D-Flow System in real-time, but to also check that their electronics installed on the detector at CERN (or other site) are working properly.
   At the remote laboratory of the university, several tests can be performed on these 256 GB (or 2 TB) of trigger raw data. For example, the data can be analyzed to check that the electronics of all 8,192 channels are generating expected data or to identify any dead channels. If an electronic channel or module is shown to be defective, the experimenters can efficiently plan a trip from their university to CERN or other accelerator site with a working module to replace the defective one.
Another advantage of having a TER/DSU unit at CERN (or another site performing similar experiments), is to use the TER/DSU unit at the accelerator-detector site to record 256 GB (or 2 T) of trigger raw data with an LHC beam at different known energies (or the detectors can be stimulated with LED light or other known sources that will generate an electrical signal from the detector of a known energy and duration in time). This recorded trigger raw data from different stimuli to the detectors at known energy values will be very useful to the scientific associates at different universities to determine the pedestal values to be subtracted for each channel and the gain they have to store in the lookup tables of each of the 8,192 channels of their electronics. This will be essential for the calibration of all parts of the instruments (CMS, Atlas, etc.) to avoid discrepancies in data such as the Higgs boson-like particle whose energy was recorded as 125.3 GeV and 126.5 GeV in different experiments.

The center column of FIG. 39 shows the 3D-Flow OPRA system for 8,192 channels capable of extracting from 8-64 million events (32-bit×8,192) arriving at a rate of 1.3 TB/sec, ALL valuable information from radiation using up to 40 steps of Object Pattern Real-Time Recognition Algorithms executed in parallel on each of the 32,768×3D-Flow processors @ $1 each (10,240×3D-Flow processors out of the total 43,008 processors in the system are used by the 3D-Flow pyramid to funnel data to a single output channel).

The right column of FIG. 39 shows the Results Analyzer Unit (RAU) which verifies that the 3D-Flow system has extracted all valuable information from radiation. (The rare particles found by the 3D-Flow system satisfying experimenters' Level-1 Trigger algorithm). See FIG. 39.

B. The 3D-Flow Invention Used in the 3D-CBS (3-D Complete Body Screening) Benefits Humanity with the Potential to Save Millions of Lives and Reduce Healthcare Costs when Used in Medical Imaging as an Early Detection Tool on Asymptomatic People and to Accurately Prognoses and Efficiently Monitor the Treatment of Many Diseases

1. A 368 GB/Sec 3-Flow System for 2304 Channels Implemented in One VME Crate with the Capability to Execute Up to 120 OPRA Steps/Event/Channel @20 MHz 64-Bit/Channel

The left column of FIG. 40 shows the 3D-CBS ER-Event Recording- and Simulator (generically known as: DSU—Detector Simulator Unit):

It provides the opportunity for any laboratory developing and improving the 3D-CBS or other PET detectors at a university or research center to have on the test bench of their laboratory a box of electronics costing approximately $25,000 providing the exact functionality of million dollar PET detector. See FIG. 40.

C. The 3D-Flow Invention Breaks the Speed Barrier in Real-Time Applications; it is Flexible, Scalable, Programmable, Modular, Technology-Independent; it can be Built in Different Platforms (e.g. VME, VXI, VPX, ATCA, μTCA, Etc.); it can Extract all Valuable Information from the Most Noisy and Rough Radiation Environment

1. A 1.3 TB/Sec 3D-Flow System for 8,192 Channels Implemented in Two VME Crates Having the Capability to Execute Up to 30 OPRA Steps/Event/Channel @80 MHz, 16-Bit/Channel

See FIG. 41 and FIG. 5.
2. the capability to execute up to 35 OPRA steps/event/channel @80 MHz, 16-bit/channel

See FIG. 42.

### BRIEF DESCRIPTION OF THE SEVERAL VIEWS OF THE DRAWING (IF ANY)

FIG. 1—Breakthrough invention. 3D-Flow OPRA—a revolutionary electronic instrument for multiple applications: advancing science, saving lives, fighting terrorism . . . . The figure illustrates 3D-Flow OPRA electronic instrument that can be implemented in a 36 cm cube of electronics, which is capable of executing pattern recognition algorithms in real-time of multidimensional objects (different ideas, or algorithms are represented as a light bulb) by analyzing all data arriving at ultra-high speed from a matrix of thousands of transducers at over 20 TB/seconds with zero dead time. It provides three examples of possible applications: a) discovering new particles (Level-1 Trigger); b) saving millions of lives and reducing healthcare costs with the 3D-CBS (3-D Complete Body Screening); and c) fighting terrorism (identifying potential threats, find a needle in a haystack)

FIG. 2—Layout of the 3D-Flow programmable system satisfying the requirements for the Level-1 Trigger of current large HEP experiments and their future upgrades. The PRAI-ATCA crate 180 in the center of the figure receives trigger raw data events from the detectors 105, 110, 115, 120 on different connectors, speeds, protocols and formats on electronic board PRAI-B 130, synchronizing them, formatting each event into 8,192 channels×16-bits and sending it via a dual backplane 135, using the board PRAI-B 140 every 25 ns, or 12.5 ns @1.3 TB/sec through 64×128 channels to the 3D-Flow system 9U×400 mm boards 410, or 420 housed in crate 490.

FIG. 3—Many crates of electronics in HEP experiments would be replaced by a single VXI 3D-Flow crate 490 providing a much more powerful tool to uncover the unknown and to confirm or exclude the existence of a subatomic particle predicted by theoretical physicists. The one crate 3D-Flow system has the capability of executing experimenters' desired programmable complex Object Pattern Recognition Algorithm (OPRA) for the Level-1 Trigger, while sustaining an input data rate over 80 million events per second from over a billion collisions per second, with zero dead-time, at a lower cost (compared to current approaches) per each good event captured.

FIG. 4—Details of the VXI implementation of 8,192 channels 3D-Flow OPRA system for Level-1 Trigger. The system extracts all valuable information using Object Pattern Real-Time Recognition Algorithms (OPRA) from 80 million events/second (radiation) at 1.3 TB/second transfer rate from over a billion collisions/second, using 43,008×3D-Flow processors @ $1 each. Data are received at the front end of the 3D-Flow OPRA boards 410 or 420 inserted in crate 490 via 512×16 Twinax (see FIG. 48 and FIG. 49) ribbon cables 145 which are soldered on a small board 645 at the receiving end of crate 490 and are soldered on a small board 646 (see FIG. 59) at the sending crate 180.

FIG. 5 show the details of the layout of the two VME crates 300, each housing 16×3D-Flow, 256 channels boards 310 or 320 connected to eight ATCA blades, each with 1024 channels received from detectors such as Atlas CMS, etc. To show the path of eight 2×16-Twinax equal length ribbon cables on each of the 64 connectors, I have used the rainbow colors to facilitate following their path. Data are received at the front end of the 3D-Flow OPRA boards 310 or 320 inserted in two VME crates 300 via 2×256×16 Twinax (see FIG. 48 and FIG. 49) ribbon cables 145 which are soldered on a small board 545 at the receiving end of crate 300 and are soldered on a small board 546 (see FIG. 58) at the sending crate 180.

FIG. 6—Details of the VME implementation of the 2,304 channels, 14,400×3D-Flow processors System for the 3D-CBS. Center figures represent the nine VME boards, 2 cm wide housed in the “3D-FLOW SYSTEM” crate connected through 2,304 Twinax ribbon cables to the “FRONT-END ELECTRONICS, A/D converters” crate. The 3D-CBS has the capability to extract ALL valuable information from radiation, reduces considerably the amount of radiation required to be administered to each patient, and enables for the first time an effective early detection of cancer and other diseases in a single examination covering all organs of the body in just four minutes. Because it gives doctors very precise information, they are better equipped to make accurate diagnoses, prognoses and efficiently monitor treatment. Additional benefits are a reduction in the cost of screening examinations and the cost of healthcare. Data are received from the “FRONT-END ELECTRONICS, A/D converters” crate to the “3D-Flow System” crate at the front end of the 3D-Flow OPRA boards 310 or 320 inserted in upper crate 300 via 72×16 Twinax (see FIG. 48 and FIG. 49) ribbon cables 145 which are soldered on a small board 545 at the receiving end of the upper crate 300 and are soldered on a small board 546 (see FIG. 58) at the sending crate 180. Bottom left represents the whole 3D-CBS detector, the detector element coupled to PMT or SiPM and the type of signals generated that are sent to the bottom “FRONT-END ELECTRONICS, A/D converters” crate.

FIG. 7—the 3D-Flow architecture from concept (left) to implementation in two FPGA 715 (Field Programmable Gate Array) to board 700 with 68×3D-Flow processors implemented in FPGA, to an ASIC 750 with 64×3D-Flow processors in a chip, to a VME board 310 with 1,600×3D-Flow processors, to the electronic system for the 3D-CBS with 14,400×3D-Flow processors in a crate 300.

FIG. 8—The 3D-Flow Logical Unit 710 assembled in layers and stack 720 architecture for a pipeline process of frames, each frame entirely processed in one processor

FIG. 9—Description of the first 4 steps of a 12 steps sequence of the 3D-Flow parallel-processing architecture for one input/output channel of a stack of 3D-Flow processors as shown in FIG. 34. On the right of the figure there is a graphic representation of the flow of the data for the first 4 steps. On the left of the figure is shown in a table the 12 steps of the flow of input data, processing time in different processors and output results.

FIG. 10—Illustration of the 12 steps sequence of the flow of the input data and output results in the 3D-Flow parallel-processing architecture for one input/output channel of a stack of 3D-Flow processors as shown in FIG. 34.

FIG. 11—Description of the last 4 steps of a 12 steps sequence of the 3D-Flow parallel-processing architecture for one input/output channel of a stack of 3D-Flow processors as shown in FIG. 34. On the right of the figure there is a graphic representation of the flow of the data for the last 4 steps. On the left of the figure is shown in a table the 12 steps of the flow of input data, processing time in different processors and output results.

FIG. 12—Illustrates the conceptual interrelation between components of the 3D-Flow system for application in medical imaging and in physics experiments (input data from different detectors, possibility to execute different algorithms, and generation of different results) and the flow of the data in the system.

FIG. 13—3D-Flow software tools: Simulator for a 3D-Flow System with thousands of processors.

FIG. 14—Photo of the 3D-Flow DAQ-DSP IBM PC modular board with 68×3D-Flow processors suitable to build 3D-Flow systems for detector of any size and proving feasibility and functionality.

FIG. 15—VME 3D-Flow single board, components on both sides, 256 channels, 1600×3D-Flow processors

FIG. 16—VME 3D-Flow single board, tentative components layout on the front of the board for a 256 channels, 1600×3D-Flow processors.

FIG. 17—VME 3D-Flow single board, tentative components layout on the back of the board for a 256 channels, 1600×3D-Flow processors.

FIG. 18—VME 3D-Flow mother board, 256 channels, 832×3D-Flow processors

FIG. 19—VME 3D-Flow daughter board, 256 channels, 768×3D-Flow processors

FIG. 20—VME 3D-Flow mother board, tentative components layout for a 256 channels, 832×3D-Flow processors.

FIG. 21—VME 3D-Flow daughter board, tentative components layout for a 256 channels, 768×3D-Flow processors.

FIG. 22—VXI 3D-Flow board, 84 ASICs, 1,024 channels, 5,376×3D-Flow processors.

FIG. 23—VXI 3D-Flow board, 40 ASICs, tentative components layout on the front of the board for a 1,024 channels, 2,560×3D-Flow processors.

FIG. 24—VXI 3D-Flow board, 44 ASICs, tentative components layout on the back of the board for a 1,024 channels, 2,816×3D-Flow processors.

FIG. 25—VXI 3D-Flow board, 68 ASICs, 1,024 channels, 4,352×3D-Flow processors.

FIG. 26—VXI 3D-Flow board 430, 66 ASICs, 512 channels, 4,224×3D-Flow processors.

FIG. 27—VXI 3D-Flow board 430, 56 ASICs, tentative components layout on the front of the board for a 512 channels, 3,584×3D-Flow processors.

FIG. 28—VXI 3D-Flow board 430, 10 ASICs, tentative components layout on the back of the board for a 512 channels, 640×3D-Flow processors.

FIG. 29—layout of the backplane carrying the connections between neighboring processors in the 3D-Flow array located in different boards. This implementation is similar for VME and VXI crates.

FIG. 30—Experimental data over half century show that we are not winning the war on cancer with a reduction of mortality rate of less than 5%, while for the heart disease for the same period was over 50%, while the cost of cancer has increased over 100 fold. The figure illustrated the path to identify the most deadly and costly calamity in the world, and the approach to take to solve the problem.

FIG. 31—Illustration why it is important to extract ALL valuable information from radiation which is related to visualizing abnormal biological processes enabling early cancer detection.

FIG. 32—3D-CBS for measuring anatomical and functional parameters

FIG. 33. The 3D-CBS on the right provides precise information on the minimum abnormal biological process with a number (top of the fraction) measured versus a number (bottom of the fraction) considered normal of the metabolic activity (or any biological process useful to the physician to identify abnormalities leading to degenerative diseases such as cancer). On the left of the figure is shown the information provided to the physicians from current PET.

FIG. 34—3D-CBS Logical Design with its functions split for engineering them into hardware.

FIG. 35—Technological advantages of the 3D-CBS compared to current PET

FIG. 36—The 3D-CBS a single examination replacing mammogram, PAP-Test, colonoscopy and PSA examination.

FIG. 37—3D-CBS Physical layout and electronics crate

FIG. 38—Illustrates the physical layout of the different components from the DSU unit 205 generating radiation data recorded from a PET detector 102 in the left column, the 3D-Flow OPRA under test (crate 300 housing data processing boards 310 and the coincidence board 360) with the task to extract all valuable information from the radiation data (tumor markers) in the center column and the RAU unit 240 to the right, analyzing the results found and measuring the efficiency of the system. The lower layer from left to right shows the components related to the application of improving medical imaging.

FIG. 39—3D-FLOW VERIFIABLE SYSTEM for 8,192 Channels 20 OPRA steps/16-bit-channel @ 80 MHz. Detector Simulator (see crate 200 housing boards 210 or 220 using SODIMM 205. See FIG. 41, FIG. 42, FIG. 43, FIG. 44, FIG. 48, FIG. 49, FIG. 50, FIG. 51, FIG. 52) with the capability to generate up to 131,072-bit/event sent at 1.3 TB/sec transfer rate to the 3D-Flow System. 3D-Flow System crate 410 (center) housing data processing boards 410 or 420 and the channel reduction board 460 extracts all valuable information from radiation events arriving every 12.5 ns from 8,192 detector channels, 16-bit/channel, executing max 20 OPRA steps/event. Result Analyzer unit 215 in crate 240 (right) verifying all events containing valuable information have been extracted from radiation by the 3D-Flow System.

FIG. 40—3D-FLOW VERIFIABLE SYSTEM for 2,304 Channels, 120 OPRA steps/64-bit-channel @ 20 MHz. Detector Simulator (see crate 200 housing boards 210 or 220 using SODIMM 205. See FIG. 41, FIG. 42, FIG. 43, FIG. 41, FIG. 44, FIG. 45, FIG. 46, FIG. 47) with the capability to generate up to 32,544-bit/event sent at 368 GB/sec transfer rate to the 3D-Flow System. 3D-Flow System crate 300 (center) housing data processing boards 310 or 320 and the channel reduction board 360 extracts all valuable information from radiation events arriving every 50 ns from 2,304 detector channels, 64-bit/channel, executing max 120 OPRA steps/event. Result Analyzer unit 218 in crate 240 (right) verifying all events containing valuable information have been extracted from radiation by the 3D-Flow System.

FIG. 41—3D-FLOW VERIFIABLE SYSTEM for 8,192 Channels, 30 OPRA steps/16-bit-channel @ 80 MHz. Detector Simulator (left) generating 131,072-bit/event sent at 1.3 TB/sec transfer rate to the 3D-Flow System. 3D-Flow System (center) extracts all valuable information from radiation events arriving every 12.5 ns from 8,192 detector channels, 16-bit/channel, executing max 30 OPRA steps/event. Result Analyzer unit (right) verifying all events containing valuable information have been extracted from radiation by the 3D-Flow System.

FIG. 42—3D-FLOW VERIFIABLE SYSTEM for 8,192 Channels, 35 OPRA steps/16-bit-channel @ 80 MHz. Detector Simulator (see crate 200 housing boards 210 or 220 using SODIMM 205. See FIG. 41, FIG. 42, FIG. 43, FIG. 44, FIG. 45, FIG. 46, FIG. 47) with the capability to generate up to 131,072-bit/event sent at 1.3 TB/sec transfer rate to the 3D-Flow System. 3D-Flow System crates 495 (center) housing data processing boards 430 and the channel reduction board 460 extracts all valuable information from radiation events arriving every 12.5 ns from 8,192 detector channels, 16-bit/channel, executing max 35 OPRA steps/event. Result Analyzer unit 215 in crate 240 (right) verifying all events containing valuable information have been extracted from radiation by the 3D-Flow System.

FIG. 43—Specification logical drawing VME ER/DSU board using Altera FPGA for a 320 MHz, 512-bit long word, DDR: 640 Mbps×512=40.96 GB/sec Transfer rate.

FIG. 44—Specification and tentative layout of the components on the PCB for the 320 MHz VME ER/DSU board (front of the board).

FIG. 45—Specification and tentative layout of the components on the PCB for the LHC TER/DSU board (back of the board).

FIG. 46—Specification logical drawing VME ER/DSU board using Xilinx FPGA for a 640 MHz, 512-bit long word, DDR: 1280 Mbps×512=81.92 GB/sec Transfer rate.

FIG. 47—Specification and tentative layout of the Altera FPGA components on the PCB for the VME ER/DSU board (back of the board).

FIG. 48—Micro Twinax 2-16 ribbon ribbon cable 145 specifications.

FIG. 49—Micro Twinax ribbon cable 145 performance data and insertion loss & return loss graphs.

FIG. 50—Assembly details of the eight Micro Twinax 16×2 ribbon cables to the small boards at both ends of the ribbon, which house SamTec SEAF-40-03.5-S-10-2-A, 1.27 mm pitch connector on each board. The small board is 59.62 mm wide and 70 mm long. It has pads on one side, at one end of the small PCB to accommodate the 400-pin connector. At the other end of the small PCB on both sides has two columns and two rows of 16×2 pads to solder four Micro Twinax 16×2 ribbons. The connector is secured with four screws to the larger application PCB.

FIG. 51—Pin assignment for SEAM connectors at 36 Gbps LVDS signals

FIG. 52—SamTec SEAF8, 0.80 mm pitch connectors specifications

FIG. 53—Pin assignment of the LVDS signals to SamTec connector SEAM-40-03.5-S-10-2-A.

FIG. 54—Details of how the eight Micro Twinax 16×2 ribbon cables are assembled to the small boards at both ends of the ribbon which house SamTec SEAF8-40-05.0-S-10-2-K, 0.80 mm pitch connector on each board. The small board is 37.84 mm wide and 75 mm long. At one end of the small PCB there are pads on one side to accommodate the 400-pin connector. At the other end of the small PCB on both sides are four columns of 16×2 pads to solder four Micro Twinax 16×2 ribbons. The connector is secured with four screws to the larger application PCB. The eight 16×2 ribbons and the small PCB are tightened together with a strain reliever and this assembly is tightened to the larger application PCB with a second strain reliever. Connectors SEAM8-40-S02.0-S-10-2-K mating with SEAF8-40-05.0-S-10-2-K have a stacking height of 6.9 mm, mounted 80 mm from the edge of the larger application PCB board.

FIG. 55—Details of how the eight Micro Twinax 16×2 ribbon cables are assembled to the small 90° shaped boards at both ends of the ribbon which house SamTec SEAF8-40-05.0-S-10-2-K, 0.80 mm pitch connector on each board. The small 90° shaped board is 37.84 mm wide and 100 mm long along the connector end and 62 mm along the ribbon cable end. At one end of the small PCB there are pads on one side to accommodate the 400-pin connector. At the other end at 90° with respect to the connector, on both sides there are four columns of 16×2 pads to solder four Micro Twinax 16×2 ribbons. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 56—Details of how the eight Micro Twinax 16×2 ribbon cables are assembled to the small boards at both ends of the ribbon which house a SamTec SEAF8-40-05.0-S-10-2-K, 0.80 mm pitch connector on each board. The small board is 38 mm wide and 128 mm long. At one end of the small PCB there are pads on one side to accommodate the 400-pin connector. At the other end of the small PCB on both sides at 90° with respect to the connector there are two columns of two rows of 16×2 pads to solder four Micro Twinax 16×2 ribbons at 90° with respect to the connector. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 57—Details of how the eight Micro Twinax 16×2 ribbon cables are assembled to the small boards at both ends of the ribbon which house SamTec SEAF8-40-05.0-S-10-2-K 0.80 mm pitch connector on each board. The small board is 38 mm wide and 180 mm long. At one end of the PCB there are pads on one side to accommodate the 400-pin connector. At the other end of the small PCB, on both sides at 90° with respect to the connector is one column of four rows of 16×2 pads to solder four Micro Twinax 16×2 ribbons at 90° with respect to the connector. The connector is secured with four screws to the larger application PCB. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 58—Details of how the eight Micro Twinax 145 16×2 ribbon cables are assembled to the small boards at both ends of the ribbon which house SamTec SEAF8-40-05.0-S-10-2-K 0.80 mm pitch connector on each board. The small board 545 and 546 is 38 mm wide and 210 mm long. It has a cut on one side of the PCB board as long as the width of the four ribbon cables to create a window on adjacent PCBs assemblies where the four ribbon cables can cross from one side of the PCBs to the other side. At one end of the small PCB there are pads on one side to accommodate the 400-pin connector. At the other end of the PCB, on both sides at 90° with respect to the connector is one column of four rows of 16×2 pads to solder four Micro Twinax 16×2 ribbons at 90° with respect to the connector. The connector is secured with four screws to the larger application PCB. The side of the small PCB board opposite the connector has a hole at the corner in order to secure the board to a frame relieving any mechanical strain on the connector, on the four screws tightening the connector to the board, and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 59—Details of how the eight Micro Twinax 145 16×2 ribbon cables are assembled to the small boards at both ends of the ribbons which house SamTec SEAF8-40-05.0-S-10-2-K 0.80 mm pitch connector on each board. The small board 645 and 646 is 38 mm wide and 300 mm long. It has a cut on one side of the PCB board as long as the width of the four ribbon cables to create a window on adjacent PCBs assemblies where the eight ribbon cables can cross from one side of the PCBs to the other side. At one end of the small PCB there are pads to accommodate the 400-pin connector. At the other end of the PCB at 90° with respect to the connector there is one column of eight rows of 16×2 pads to solder eight Micro Twinax 16×2 ribbons at 90° with respect to the connector. In one small PCB the eight ribbons are soldered on the same side of the connector, while on the other board they are soldered on the side opposite the connector. The connector is secured with four screws to the larger application PCB. The side of the small PCB board opposite the connector has a hole at the corner in order to secure the board to a frame to relieve any mechanical strain on the connector, on the four screws tightening the connector to the board and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 60—Details of how the eight Micro Twinax 16×2 ribbon cables are assembled to the small boards at both ends of the ribbon which house SamTec SEAF8-40-05.0-S-10-2-K, 0.80 mm pitch connector on each board. The small board is 38 mm wide and 300 mm long. It has a cut on one side of the PCB board as long as the width of the four ribbon cables to create a window on adjacent PCBs assemblies where the eight ribbon cables can cross from one side of the PCBs to the other side. At one end of the small PCB there are pads to accommodate the 400-pin connector. At the other end of the PCB at 90° with respect to the connector is one column of eight rows of 16×2 pads to solder eight Micro Twinax 16×2 ribbons at 90° with respect to the connector. In one small PCB the eight ribbons are soldered on the same side of the connector, while on the other board they are soldered on the side opposite the connector. The connector is secured with four screws to the larger application PCB. The side of the small PCB board opposite the connector has a hole at the corner in order to secure the board to a frame to relieve any mechanical strain on the connector, on the four screws tightening the connector to the board, and to hold the weight of the ribbon cables. The eight 16×2 ribbons and the small PCB are tightened together with a strain reliever. To increase the number of I/O channels to the larger PCB board, connector SEAF8-40-05.0-S-10-2-K should be assembled at both ends of the small PCB connected to the ribbon cables; while at the edge of the larger application PCB board the mating connector SEAM8-40-S02.0-S-10-2-K should be assembled providing a stacking height of 7.0 mm; while at 25 mm from the edge of the larger application PCB board the mating connector SEAM8-40-S05.0-S-10-2-K should be assembled providing a stacking height of 10 mm.

FIG. 61—3D-CBS detector assembly 102 with a length of the detector (FOV longer than 1 m, consisting of the crystal 330 with slits of equal length, or a solid crystal 331 with no slits (cuts) with rectangular or pyramidal shape (larger face external to the cylinder, smaller face internal to the cylinder, the crystal may have a dimension larger than the sensor 324), one element SiPM 334 with a surface smaller than the crystal coupled to the inner face of the crystal through a pyramid shape light-guide, a PMT, SiPM or APD with a single sensor or an array of sensors 324 coupled to the external face of the crystal.

FIG. 62—Detail of the assembly of one electronic channel of the detector consisting of an APD or SiPM 334 coupled to the inner face of the crystal through a pyramid shape light-guide and an outer sensor 324 (PMT, SiPM or APD) coupled to the outer face of the crystal. The detector may consist of a crystal 330 with slits of equal length, or a solid crystal 331 with no slits (cuts) with rectangular or pyramidal shape (larger face external to the cylinder, smaller face internal to the cylinder, the crystal may have a dimension larger than the sensor 324), one element SiPM 334 with a surface smaller than the crystal coupled to the inner face of the crystal through a pyramid shape light-guide, a PMT, SiPM or APD with a single sensor or an array of sensors 324 coupled to the external face of the crystal.

FIG. 63—Detail of the same crystal assembly as in FIG. 62, showing 330 crystals with slits (cuts) of equal length, with adjacent crystals and the center of gravity algorithm for better spatial resolution.

FIG. 64—Different shapes and lengths of the 3D-CBS detector optimized for low cost and maximum efficiency.

FIG. 65—Photon detection programmable algorithm with the 3D-Flow processor. Each processor gathers the information from its detector element and the 8 neighbors and acts like the head of a cluster without boundary limitation. Any further operations can be executed upon the 9 data (the one received from the detector and its 8 neighbors) by the CPU of the 3D-Flow processor, which can, in a single cycle, execute up to 26 operations, including all normal arithmetic and logic operations from a standard computer to greatly improve signal-to-noise ratio.

FIG. 66—Photon detection programmable algorithm with the 3D-Flow processor. Each processor gathers the information from its detector element and the 24 (5×5) neighbors and acts like the head of a cluster without boundary limitation. Any further operations can be executed upon the 25 data (the one received from the detector and its 24 neighbors) by the CPU of the 3D-Flow processor, which can, further improve signal-to-noise ratio.

FIG. 67—Specification of the electronics of the alternative Explorer project with limited efficiency and higher cost than the 3D-CBS

FIG. 68—Specification of the detector and sensors assembly of the alternative Explorer project with limited efficiency and higher cost than the 3D-CBS

FIG. 69—Comparison table between the Explorer project and the 3D-CBS

FIG. 70—Estimated lives saved and variable revenue plan for 30 years for the MasSpec Pen

FIG. 71—Timeline for the development of the 3D-Flow OPRA and the 3D-CBS systems

FIG. 72—Estimated lives saved and variable revenue plan for 30 years for the 3D-CBS (3-D Complete Body Screening).

### DETAILED DESCRIPTION OF THE INVENTION

A. The Revolutionary Scientific Advantages of the 3D-Flow Invention which Extracts All Valuable Information from Radiation when Applied to HEP Experiments

The 3D-Flow System provides a very powerful tool to the HEP community because it extracts all valuable information from radiation.

In HEP applications, the 3D-Flow architecture provides data exchange with neighboring processors while its bypass switch allows the execution of uninterruptable algorithms for a time longer than the interval between two consecutive input data. The result is it can execute experimenters' desired programmable complex Object Pattern Recognition Level-1 Trigger Algorithms (OPRA), while sustaining an input data rate of over 80 million events per second from over a billion collisions per second, with zero dead-time, at a lower cost per each good event captured than other approaches.

Many physics experiments would have already benefitted from this powerful tool to discover new particles and save a great amount of money if DOE had provided the NRE cost in 1995 to build the 3D-Flow chip after funding its study. After receiving $906,000 from the DOE in 1995 and paying Synopsys, one of the best companies designing integrated circuits that generated the RTL files for the silicon foundry to build 4×3D-Flow processors in 350 nanometer technology in a chip, funding went to more costly projects with lower performance. My 3D-Flow invention was recognized valuable by many scientists who wrote letters of appreciation after 1992. In 1993, it underwent a formal scientific review at FERMILab gaining further recognition. In 1994, it was recognized by DOE as a breakthrough invention benefitting science and published on page 216 in the DOE Technology Transfer book DOE/LM-0002 DE94005148. In 2001 it was proven feasible and functional in hardware in 2×FPGAs (Field Programmable Gate Array), each containing 4×3D-Flow processors, interconnected in a cube 2×2×2 structure. In 2003 it was proven feasible and functional in two modular industrialized boards, each with 68×3D-Flow processors in 17 FPGA, interconnected within the board in a (4×4×4) cube structure+1 FPGA of 4 processors for channel reduction. The successful testing of the communication between the two modular boards proved that 3D-Flow systems can be built for detectors of any size.

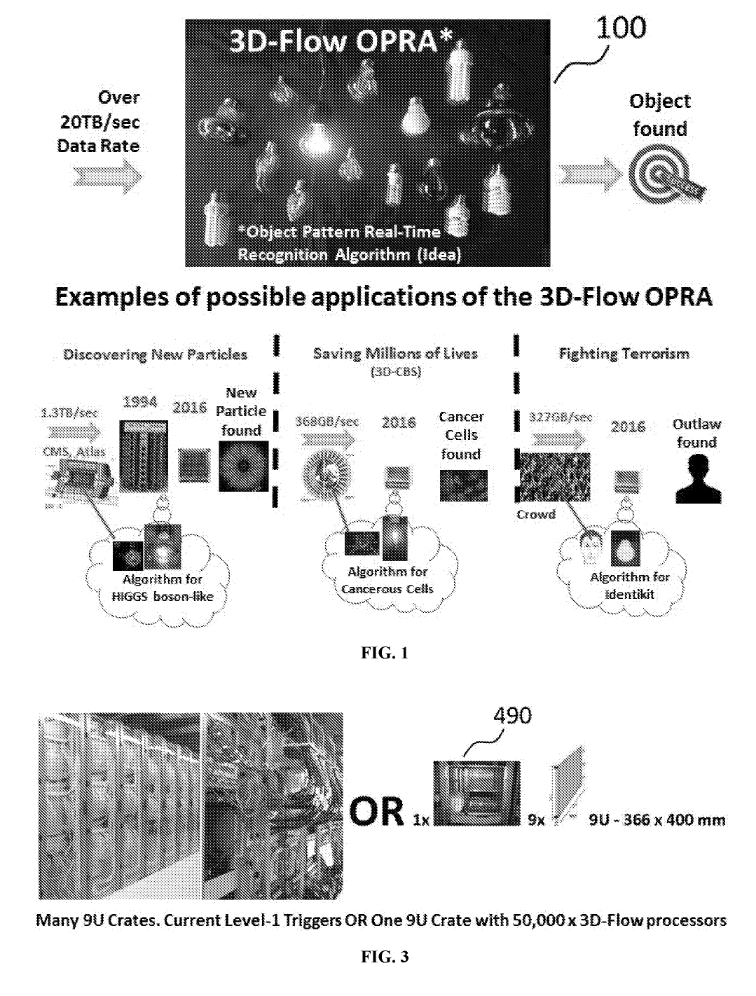

The entire Level-1 programmable system of over 40,000×3D-Flow processors at $1 each can fit into a single crate. A patch panel PRAI-ATCA (see FIG. 1) receives events from the detectors, synchronizes and formats each event into 8,192 channels×16-bit and sends them to the 3D-Flow system—one every 12.5 ns (or 1 every 25 ns with a longer 32-bit word).

Because the LHC collider and experiments cost billions of dollars and the Level-1 Trigger has the most important and challenging task of capturing rare events (one out of 10 billion in the case of the Higgs boson-like particle), if they are missed all the work of analyzing data by thousands of people will be meaningless and the entire money and effort of building for more than 20 years the LHC collider and detectors would be wasted. It would therefore be prudent to thoroughly test the efficacy of the Level-1 Trigger and give the opportunity to more than one group of experimenters/scientists to test their ideas.

In order to allow more than one group of scientists within an experiment to implement and test their Level-1 Trigger algorithms and hardware, a suggestion would be for scientific committees which approve experiments and funding agencies in Europe, U.S. and other countries participating at CERN experiments as member states, associates or observers to create opportunities for open PUBLIC fair scientific reviews where scientists and inventors can submit their ideas for funding, implementation and testing a new Level-1 Trigger on LHC experiments.

Level-1 Triggers of the large experiments at LHC: CMS Atlas, Alice and LHCb do not provide zero dead-time; CMS and Atlas have found only 40 Higgs boson-like events during analysis of data captured randomly instead of intelligently, by the Level-1 Trigger; their leaders recognize they must trash the current Level-1 Trigger electronics because they do not have the capability to execute Object Pattern Recognition Algorithms.

What we should learn from these past errors is that an investment of over $50 billion and 25 years of work by 10,000 people should not be left to a single Level-1 Trigger group in CMS and another in Atlas who did not compete on merit scientific ideas in a fair PUBLIC scientific review with different projects. If such a merit review had been conducted, the 3D-Flow with OPRA capabilities and zero dead-time recognized by a formal public scientific review at FERMILab in 1993 should have been adopted by the LHC experiments.

Because the current Level-1 Trigger has not provided accurate measurements about the unknown particle predicted in theory, helping to rule out or confirm theoretical predictions, CMS and Atlas will continue to claim that more data needs to be acquired to fully understand the nature of the new found particle. However, if this rare event occurs one time in 10 billion events and the Level-1 Trigger does not work effectively, casually capturing some of them amounts to pure luck not experimental science, takes a long time, and is like waiting to win the lottery.

Instead, it will be worth having four or more 3D-Flow OPRA systems, one installed in the experiment at CERN and three on test benches at remote universities and other laboratories to test experimenters' desired real-time algorithm on a real 3D-Flow OPRA system.

The cost of $98,000 for the main frame 3D-Flow system, $40,000 for the DSU (Detector Simulator Unit sending LHC recorded data and difficult to recognize, special events with pileup signals edited manually to the 3D-Flow System) and $10,000 for 8,192 Twinax cables and connectors as detailed in the design below and in the quote #34a) for a total cost of approximately $200,000×4 systems (one at CERN and 3 installed at remote universities)=$800,000 will be well justified to reduce the risk of wasting an investment of over $50 billion and 25 years work by over 10,000 people, by failing to capture data satisfying experimenters' desired complex algorithm.

Its faster, powerful OPRA filtering capability with 43,008×3D-Flow processors compared to FPGA can thoroughly analyze all data within the 2 to 3 microseconds allocated for the Level-1 Trigger and make up for the lost time buffering all data from the official Level-1 Trigger system selected by the scientific committee.

1. The Big Picture: Assessing the Future Need of Instrumentation in High Energy Physics

The increase in power at the LHC Collider poses a challenging task in assessing how performant the instrumentation should be, in particular Level-1 Trigger, responsible for making the first very important decision of which events among the trillions of events generated by the LHC, have important information valuable enough to capture. If Level-1 Trigger does not have the capability to capture these valuable events, then thousands of scientists will analyze garbage data and billions of dollars and many years of work will be wasted.

The requirements in 1994 for the Level-1 Trigger were defined in 8,000 channels, each transferring 10-bit of information every 25 ns. This was confirmed recently on page 79, Section 7.2.1 of the CMS 2013 upgrade.

This proposal is providing instrumentation satisfying those requirements and going beyond them to satisfy requirements for future LHC upgrades.

I noticed in several official documents that CERN is moving toward the standardization of 10 Gbps links. I will be glad to comply with this standardization all the way from the CMS, Atlas, Alice and LHCb detectors to the instrumentation analyzing this data with the 3D-Flow OPRA system. It is just a matter of cost, but as I have cost-effectively designed the cables and connectors at $1.17 per channel listed in item 60 on page 152 of the Budget Justification satisfying 10 Gbps speed, I could design the other parts of the system complying with 10 Gbps.

However, it is not the transfer speed which is the real issue. If we need to transfer 10 Tbps Trigger data, whether we use 8,192 cables at 1.28 Gbps, 1,048 cables at 10 Gbps, or 105 cables at 100 Gbps is determined by the cost of each approach. The current components (cables, connectors, SERDES) may offer a more cost-effective solution now at one speed, but tomorrow's market may offer other components.

What is important to address instead is the amount of trigger data generated by each detector that needs to be processed by the Trigger instrument (3D-Flow OPRA or FPGA). From now until 2018 it is the number of bits generated by each of the 8,000 (or 8,192) Trigger Towers 108 every 25 ns which is 10-bits. This generates 3.2 Tbps of data.

Whether the granularity of the Level-1 Trigger is increasing or the LHC bunch-crossings or detectors are added to the Level-1 trigger such as the Inner tracker, the amount of data to be processed at the Level-1 Trigger will increase; however, it is unlikely that the LHC bunch-crossings will increase over 40 MHz until the beam heating problem is solved.

If the Trigger Tower 108 granularity is increased from 8,192 channels to 32,768, one could use 4× crates, each with 8,192 channels and 43,000×3D-Flow processors; however, the OPRA algorithm will increase in complexity (will be longer) because each processor will have to handle more neighboring data from the impact of the particle in the detector, affecting more elements in a smaller granularity.

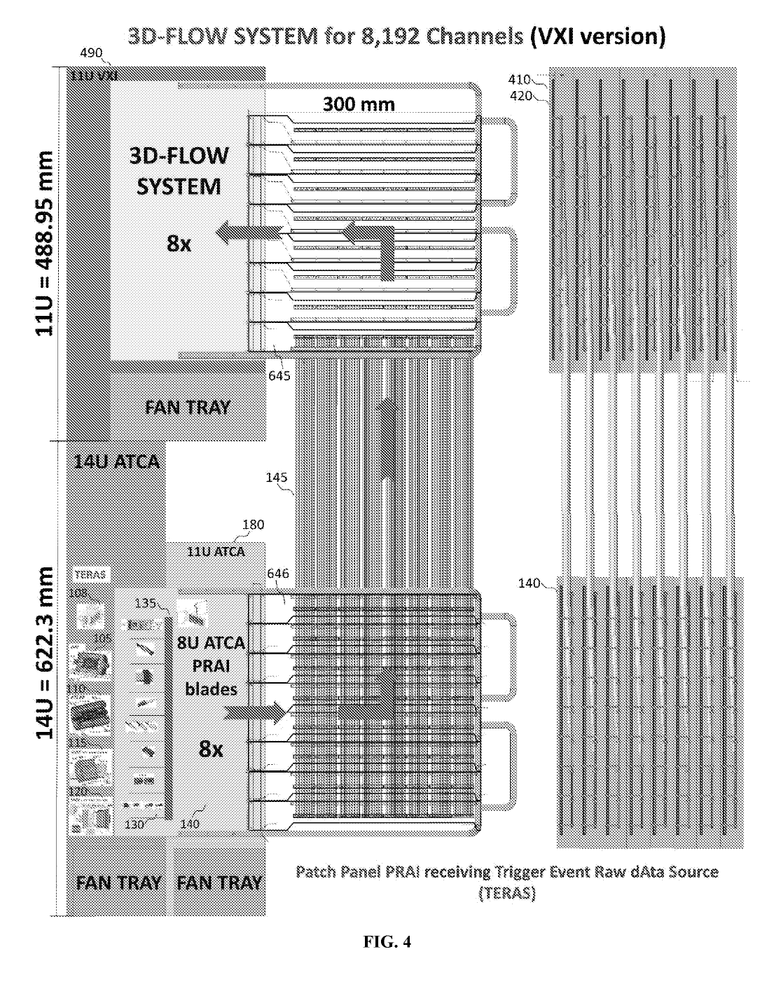

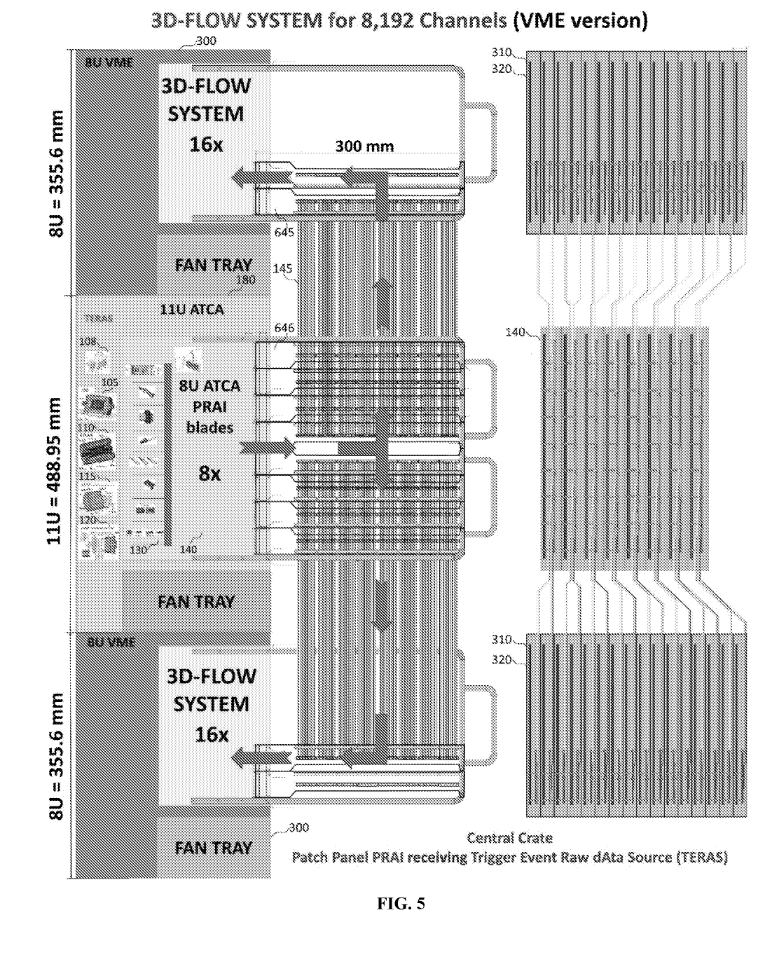

If the number of detectors is increased and instead of generating 10-bit every 25 ns, the Trigger Towers 108 in FIG. 2, FIG. 4 and FIG. 5 generates 16-bits, 32-bits, 64-bits, . . . then the Level-1 trigger should have the capability to process more data.

To simplify this study, let's suppose that the LHC bunch-crossing will not increase over 40 MHz and the 10-bit per channel increases to cope with higher luminosity and more detector information at Level-1 Trigger to:
1. Level A “16-bit per Trigger Tower every 25 ns” until 2018 (already an improvement over the current 10-bit information from each Trigger Tower)
2. Level B “32-bit per Trigger Tower every 25 ns” beginning 2018
3. Level C “250-bit per Trigger Tower every 25 ns” to comply with CERN 10 Gbps standard

As I mentioned before, the 3D-Flow OPRA new instrument can satisfy any additional requirements in a very cost-effective manner. It is important to assess the future needs of instrumentation in HEP in order to optimize the cost-performance.

The proposed project and budget to build the 8,192 channel 3D-Flow OPRA system described in FIG. 4 will achieve Level A and Level B.

To facilitate the understanding of cost-performance, I have divided the cost into four main categories:
a) cost of “instrumentation”
b) cost of “cables+connectors”
c) cost of the “3D-Flow OPRA”
d) cost of the “LHC TER/DSU”

To save money until 2018, groups of experimenters who have a small budget and need to have good instrumentation to test the Level-1 Trigger on the test-bench of their lab can add their order to this proposal for a 640 Mbps LHC TER/DSU for about $40,000. This item complies with Level A.

To upgrade the performance of the entire Level-1 trigger system to Level B, it will only be necessary to have the 1.28 Gbps LHC TER/DSU costing about $120,000.

The 8,192 cables+connectors of this proposal, for a total cost of $10,000 to transfer up to 82 Tbps complies with Level C.

To bring the four items listed earlier into compliance with Level C will require the following:
a) “instrumentation” Item 6 for about $500K and item 8 for about $100K of the Budget Justification spreadsheet should be purchased instead at the current oscilloscope price of $64 k of item 7.
b) “cable+connectors” will remain the same with a total cost $10,000
c) “3D-Flow OPRA” will need to change to a technology supporting SERDES at 10 Gbps input and output. This should be addressed in depth with ASIC designers. During an informal conversation I was told that 10 Gbps was certainly feasible but the cost of the NRE to the foundry could quadruple.
d) “LHC TER/DSU” should increase its speed from 1.28 Gbps per channel to 10 Gbps per channel. This will also increase the cost and is the reason why I have designed a version at 640 Mbps and, one at 1.28 Gbps; a higher speed version is justified only if required by the other instruments.

Each 3D-Flow processor can execute programmable, complex Object Pattern Real-Time Recognition Algorithms by exchanging data with neighboring processors and manipulating any field or group of bits of the 16-bit, 32-bit, 64-bit . . . or 250-bit word with OPRA steps (OPRAS), each with the capability to execute up to 26 operations such as, addition, subtraction, comparison with 24 values, etc. in less than 3 nanoseconds.

Although the 3D-Flow architecture can satisfy experimenters' requirements to execute longer algorithms when the input word is increased from 16-bit to many more bits by adding the number of 3D-Flow processor layers, do we really need 10 Gbps to carry 250-bit information from each Trigger Tower or is a compromise between 32-bit of the proposed 3D-Flow processor with 1.28 Gbps input/output and 10 Gbps acceptable? Perhaps a 2.56 Gbps offering 64-bit word input from each Trigger Tower would be sufficient and the 3D-Flow processor NRE at the Silicon Foundry will not quadruple.

This proposal offers a set of instruments that are cost-effective, flexible and scalable to satisfy increasing requirements. It will be important to address the issue of whether the information from the Trigger Towers in 2020, after additional detectors are added to cope with increased luminosity, will exceed 32-bits every 25 nanoseconds, rather than to impose a standard to transfer 10 Tbps.

Whether 10 Tbps are transferred with 8,192 cables at 1.2 Gbps, 1048 cables at 10 Gbps or 105 cables at 100 Gbps should be determined by the most cost-effective solution commercially available, but this will have no effect on its ability to find a new particle—one solution just costs more or less than another.

2. Saving Taxpayer Money: The 3-Flow System can Replace Many Crates of Electronics in HEP Experiments with a Single Crate, Providing a Much More Powerful Tool to Discover New Particles or to Disprove a Theory

The most difficult task that I have to face for two decades is to make leaders of large experiments understand the importance of creating a tool capable of extracting ALL valuable information from radiation with zero dead-time. This means to thoroughly analyze at the Level-1 Trigger decision, ALL events' raw data received directly from the detector using programmable complex Object Pattern Real-Time Recognition Algorithms (OPRA) for a time longer than the interval between two consecutive events.

Although the 3D-Flow invention having these capabilities was recognized valuable by academia, industry and the world's most prestigious research centers in a formal scientific review, leaders of large experiments failed to recognize its benefits, funding instead less efficient and more costly approaches that could not thoroughly analyze each event without zero dead-time.

These other approaches are documented in at least 12 volumes of proceedings, about 600 pages each, generated during 20 years of “Workshops on Electronics for LHC and Future experiments” (e.g. CERN-2007-001, CERN-LHCC-2007-006, LHCC-G-125, 15 Jan. 2007, the “12^{th} Workshop on Electronics for LHC . . . ”), and in several volumes, some 600 pages each, generated by the Trigger groups during 23 years of large experiments at CERN, such as the 600-page “The Trigger and Data Acquisition project, Volume 1, The Level-1 Trigger, Technical Design Report” CERN/LHC 2000-038, CMS TDR 6.1, 15 Dec. 2000, their upgrades in 2013, and the proceedings from several conferences including the 23 annual IEEE-NSS-MIC conferences, one of which (2013) generated over 10,000 pages of proceedings as reported at this link to its abstracts. The electronics described in these documents costing hundreds of millions of dollars was the result of over 10,000 scientists costing additional billions of dollars in salaries and equipment being lead in the wrong direction because the Level-1 trigger did not have the capability to extract ALL valuable information from radiation. It did not have the capability to capture ALL rare events satisfying experimenters' desired characteristics of new particles.

CERN documents state that in 2011-2012, the Large Hadron Collider (LHC) at CERN generated 1,000 trillion (10¹⁵) events, 100,000 of which were estimated to be Higgs boson-like. On Jul. 4, 2012, CERN announced they had found 40 such Higgs boson-like events at each experiment, Atlas and CMS, by analyzing several terabytes of acquired data and noticing an unusual energy at 125.3 GeV in about 40 events by CMS and at 126.5 GeV for the same number of events by Atlas. These 40 Higgs boson-like events out of the estimated 100,000 indicate a casual recording of those events rather than an informed decision made by the Level-1 Trigger matching desired conditions.

The level-1 Trigger of CMS and Atlas have many shortfalls including not having the capability to capture ALL rare events satisfying experiments' desired characteristic of the new particle. Here are some facts and observations that support this statement:
1. Their current Level-1 Triggers are not dead-time free, nor are their designs for future upgrades of CMS and Atlas Level-1 Triggers. This is not only evident by examining the schematics in the previously mentioned documentation, and in the articles and documentation for future upgrade of the electronics of the Level-1 Triggers of CMS and Atlas, published and presented at conferences, but was also confirmed when I asked this question directly to the presenters of these documents at the Trigger Session of the 2013 IEEE-NSS-MIC-RTSD. A deeper investigation will show that they have very limited programmability, cannot execute complex real-time trigger Object Pattern Recognition Algorithms, and cannot extract ALL valuable information from radiation satisfying experimenters' desire to capture new particles with specific characteristics.
2. This proves that even if the hardware is working correctly, they do not have the capability to find and capture new particle with the desired characteristics satisfying experimenters' complex algorithm.
3. A further proof would be to load the trigger raw data of those 40 Higgs boson-like events with other spurious events in the LHC TER/Simulator (DSU) hardware unit. Then connect this crate to the CMS and Atlas Level-1 Trigger electronics to verify if it fires a Level-1 Trigger only for those 40 events and not for the other events.
4. The fact that the upgrade documents for CMS and Atlas state that the electronics for their Level-1 Trigger must be completely replaced is confirmation that the current system does not work.

Recently, I had the opportunity to study the state-of-the-art FPGA technology by the two major companies and their projection of the new components to be delivered next year. Without diminishing the value of FPGA technology, which has great advantages when solving problems in many fields, in this specific application its advantages do not measure up or even come close to the advantages of the 3D-Flow architecture and invention. The 3D-Flow has the capability to execute on each of the 40,000 plus processors in parallel a different programmable sequence of steps/instructions with a high-speed, short latency and low power consumption data exchange capability between adjacent processors and executing up to 26 operations such as add, subtract, compare with 24 values, etc. in less than 3 nanoseconds.

A 8,192 channels Level-1 Trigger system built using FPGA would not fit in a crate, the power consumption and cost would be exorbitant, and it would never come close to having the capability to execute Object Pattern Recognition Algorithms with the same complexity as the 3D-Flow system, directly on raw trigger data received at high speed at each of the thousands of processor nodes.

This can be proven by having funding agencies organize a meeting between myself and the designers of the other Level-1 trigger system built with FPGA where each of us provides calculations and references to technology supporting expected results to prove the global overall performance of the Level-1 Trigger system feasible.

It will be impossible for a Level-1 Trigger system built with FPGA to provide the same performance as the 3D-Flow to the experimenters' and the consequence will be that they will be limited in executing object pattern recognition algorithms, that will not be efficient in extracting ALL information from radiation, missing the rare particle and not having the capability to filter increase noise and separate pileup events at a higher LHC luminosity.

This will continue to limit experimenters' discovery of new existing particles because the Level-1 trigger cannot extract all their characteristics and likewise will not be able to disprove a theory with certainty because of the same limitations. There will continue to be inconclusive results as occurred after the announcement of the discovery of the Higgs boson-like particle on Jul. 4, 2012, when scientists stated the need to acquire more data to understand the real nature of the new particle found.

Unfortunately, instead of following scientific procedures, implementing a dialogue among scientists, showing transparency in science and responding to legitimate scientific questions, since the year 2000 the scientific community of the major conferences in the field has rejected all the papers I submitted where I have explained the importance of extracting ALL valuable information from radiation at the lowest cost per valid event captured; they have also refused a public dialogue where young scientists, PhD students and senior scientists could present their ideas and question each other publicly in two workshops that I proposed at the same conference in 2014, and continued to reject my papers this year as detailed at the Section “Recent Communications” with reviewers giving non-scientific reasons: «The abstract and summary appear to be a rehashing of work done 23 years ago. Can't tell if there is anything new or interesting. I am unaware of any experiment that has used the 3D-Flow-OPRT technology since then». or this other non-scientific reason «This is one of those topics “that won't go away”. I would like to hear a discussion about this to “once and for all” settle the question raised—is this approach any good? . . . I think it would be up to the moderators of the session whether they want to take this topic on, or just tell him (once more) to go away».

After recognizing the validity of my 3D-Flow invention in a formal scientific review in 1993, after it was acclaimed in many letters by scientists and experts in the field, after it was proven feasible and functional in hardware, after the 3D-Flow system was proven superior to any other approach yet funding went to less efficient and more costly Level-1 trigger systems, after having the proof that thousands of electronic boards and hundreds of crates of electronics of the other funded approaches failed to provide a system with the capability to extract ALL valuable information from radiation wasting taxpayers' money and the time of over 10,000 scientists who were provided with data that led to inconclusive results, after the refusal for 25 years to fund the NRE of the 3D-Flow chip and to complete the construction of a full 3D-Flow system as recognized valuable and started its funding in 1994 ($150,000) and 1995 ($906,000), after the scientific community refused to implement transparency in science and follow scientific procedures, after having prevented the presentations of my research work at the IEEE-NSS-MIC conferences since the year 2000 with the exception of when Ralph James was General Chairman in 2003, after leaders of the same conference prevented a dialogue with students, young scientists and senior scientists in two workshops where they could question each other publicly, after this last rejection of my papers with the clear desire by one of the IEEE reviewers “ . . . I would like to hear a discussion about this to “once and for all” settle the question raised—is this approach any good?”, and because the scientific community spending taxpayers' money to build the over $50 billion LHC experiments has been incapable for 23 years to implement this discussion, it would be the call and responsibility of the funding agencies that are handling taxpayers' money to organize a fair PUBLIC merit review that will make the scientific truth for the benefit of mankind emerge.

Ultimately, the final judge in science should be left to the results of an experiment but the funding agencies should ensure that tools required to measure the performance of different trigger systems are funded and built, such as the two proposed LHC TER/DSU (LHC Trigger Event Recorder and Detector Simulator Unit) and the RAU (Results Analyzer Unit) that will provide impartial measurable results among the proposed Trigger Systems. If political, personal or power-related interests cannot find a more cost-effective solution to advance science for the benefit of humanity then it would be justified to build these two Units: DSU and RAU costing less than $50,000, which would prevent $50 billion dollars and years of work by over 10,000 scientists from being wasted.

B. The Revolutionary Benefits to Humanity of Extracting All Valuable Information from Radiation Provided by the 3D-Flow Invention when Applied to Medical Imaging

One of the most difficult tasks that I have had to face for over a decade is to make reviewers of medical research funding agencies realize the importance of creating a Medical Imaging device capable of cost-effectively extracting ALL valuable information from radiation because this is key to making giant leaps in technological improvements in spatial resolution, sensitivity, lower examination cost, and lower radiation dose, that will provide staggering benefits in saving lives through an effective early detection of cancer and many other diseases when detected at a curable stage, and will help with diagnoses, prognoses, and monitoring the treatment of many diseases while reducing healthcare costs.

Researchers are led by the misconception that the most important feature of all medical devices is spatial resolution because they receive such requests from doctors, hospitals, pharmaceutical companies, cancer organizations and leaders in the field who want to measure the smallest tumor size and the minimum tumor shrinkage as a consequence of administering a drug. Consequently, they do not focus on extracting ALL valuable information from radiation but, instead throw away over 90% of the useful data from radiation using a poor geometry design that at the same time misses the signals that would provide them better information on spatial resolution.

What did the most disservice to taxpayers was when I submitted more than ten proposals for over ten years for the development of medical imaging devices having the capability to extract ALL valuable information from radiation that could have already saved many lives and reduced healthcare costs, and reviewers rejected these proposals, requesting instead that I modify my proposal for the development of PET detector modules focusing primarily on improving spatial resolution to the detriment of sensitivity.

They suggested I use the traditional economical geometry of the detector (16 cm short detector Field of View) using expensive crystals missing most of the valuable radiation rather than approving my geometry of the detector covering most of the patient's body with economical crystals to extract ALL possible valuable information from radiation (radioisotopes) at the lowest cost per valid signal captured. They still do not understand that my 3D-CBS innovative technology using the 3D-Flow architecture not only extracts all valuable information from the radiation but also maximizes the spatial resolution together with all other parameters providing great improvements in the features of medical imaging devices.

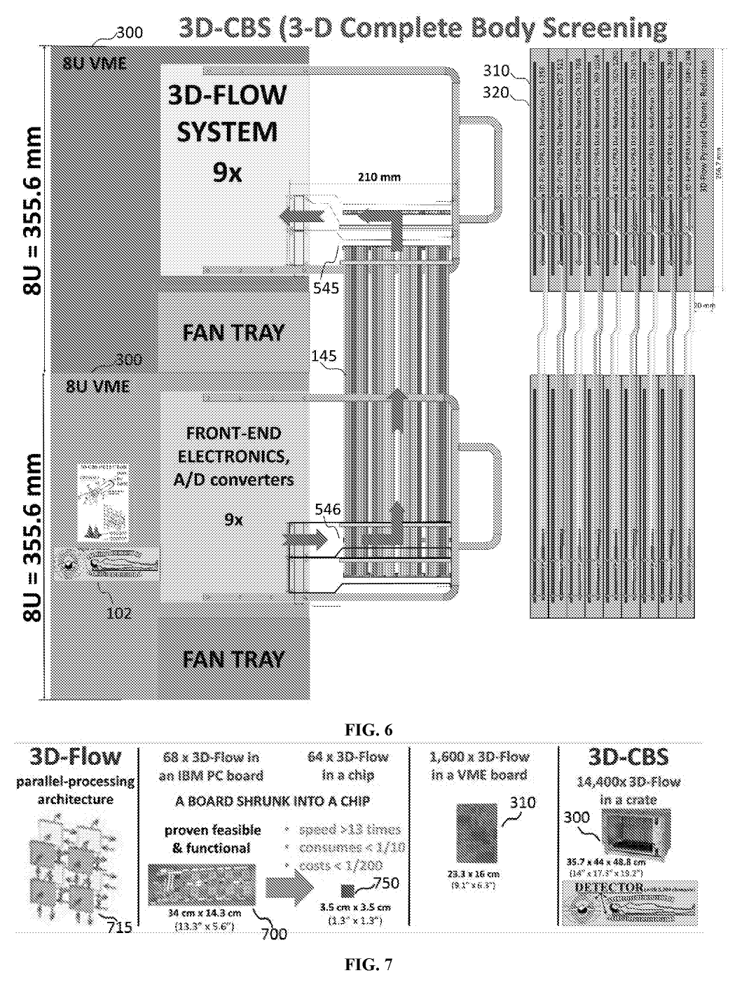

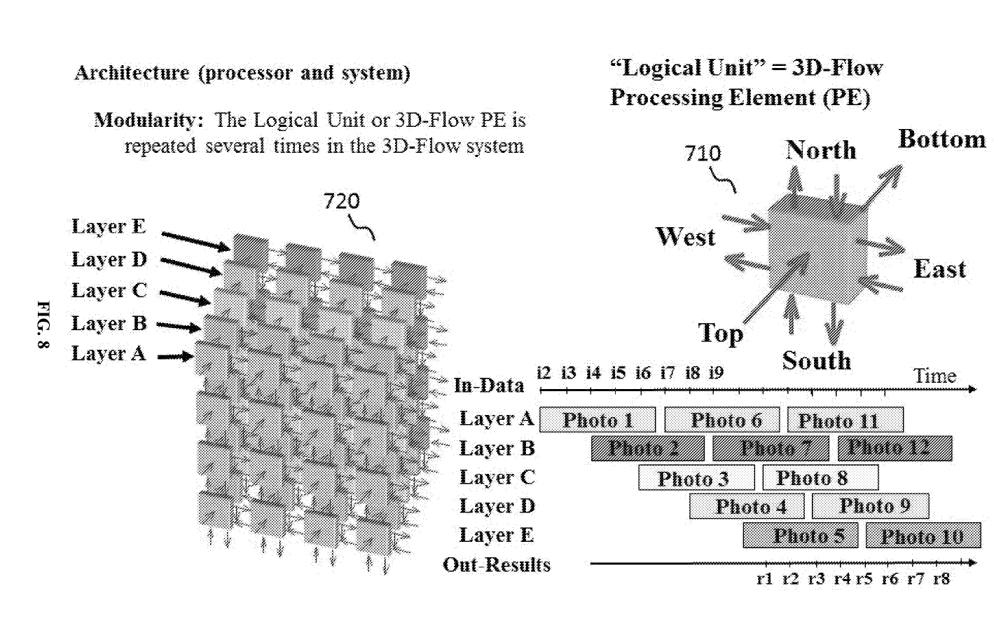

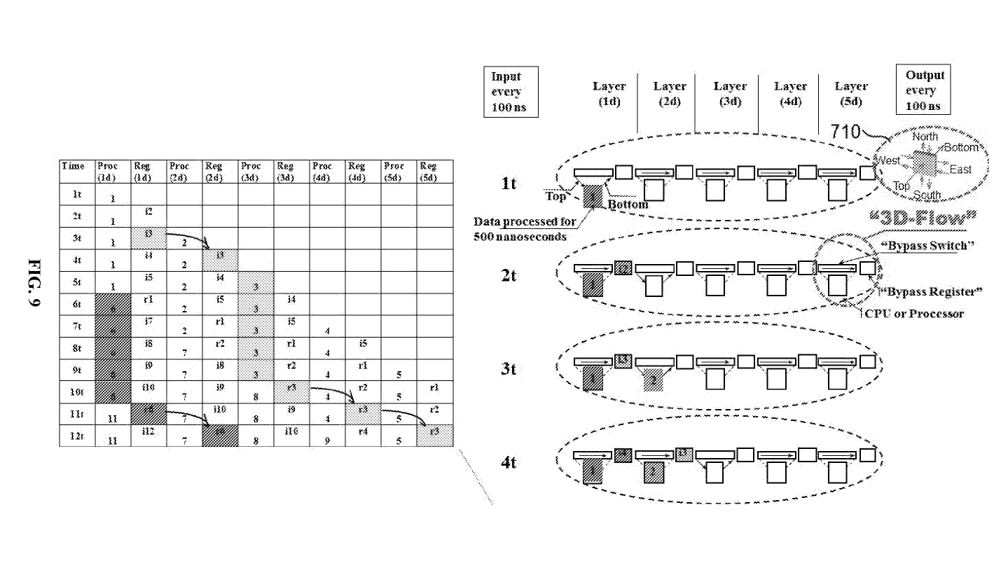

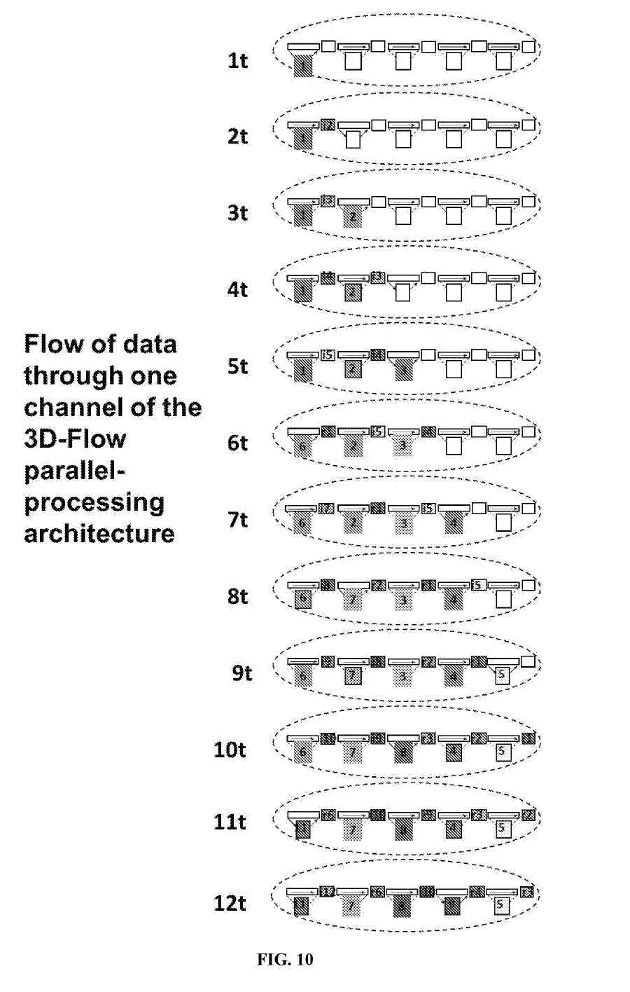

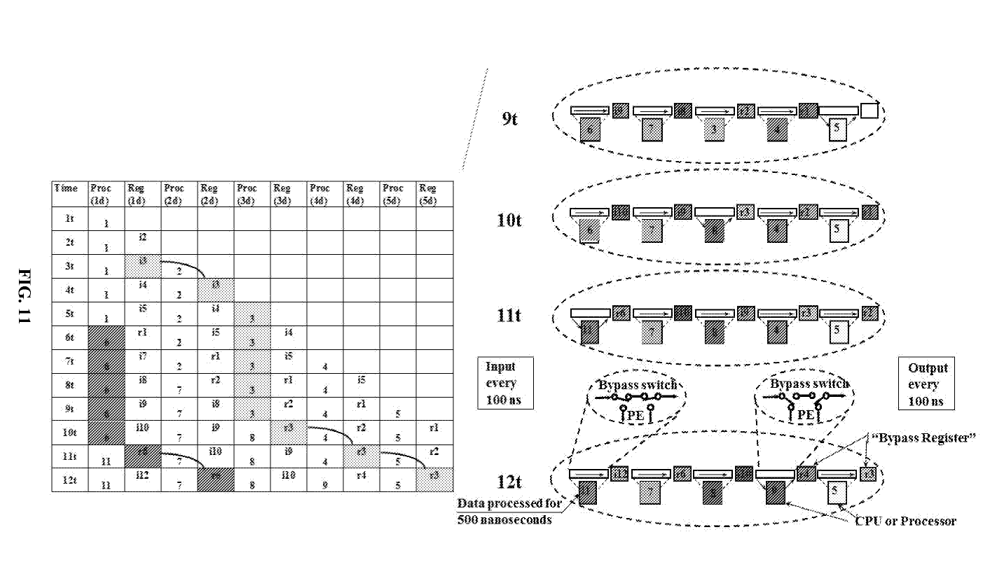

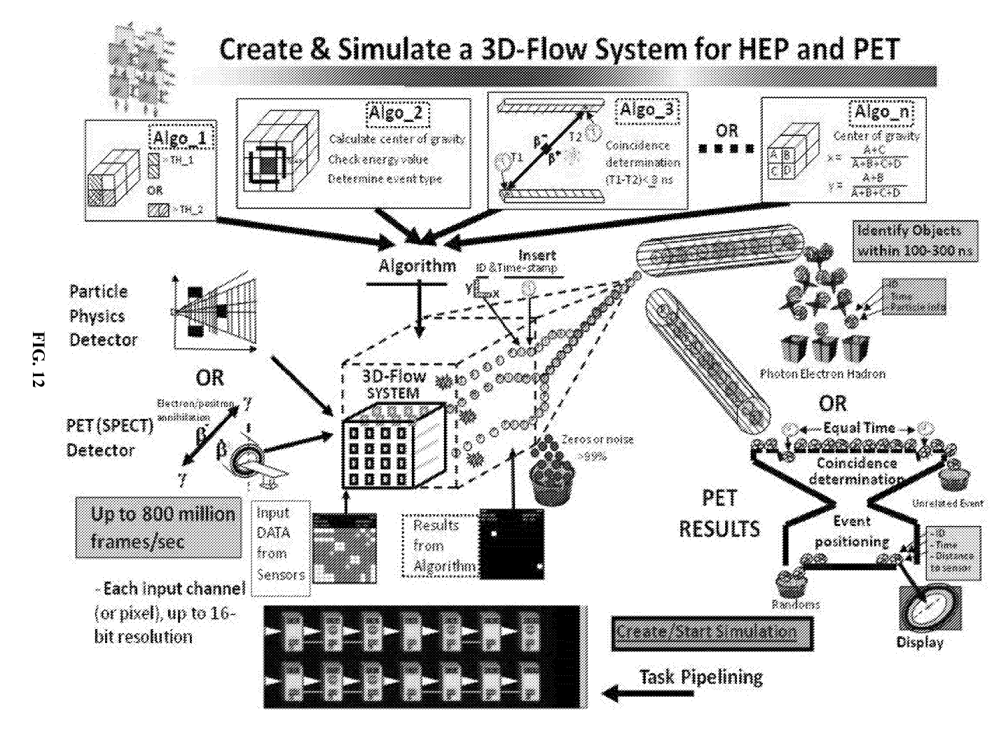

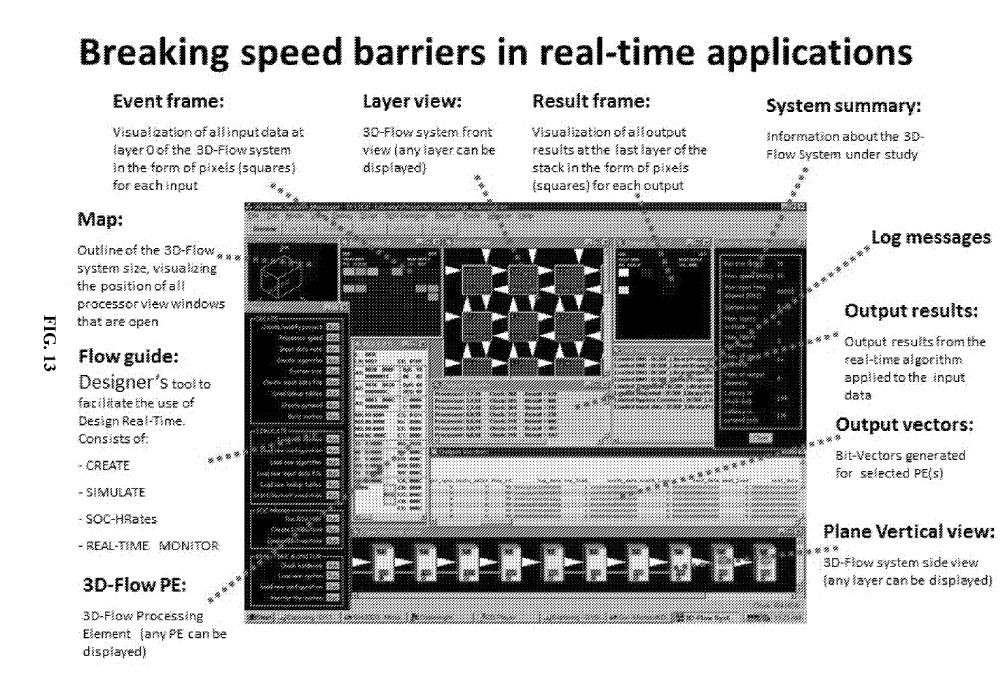

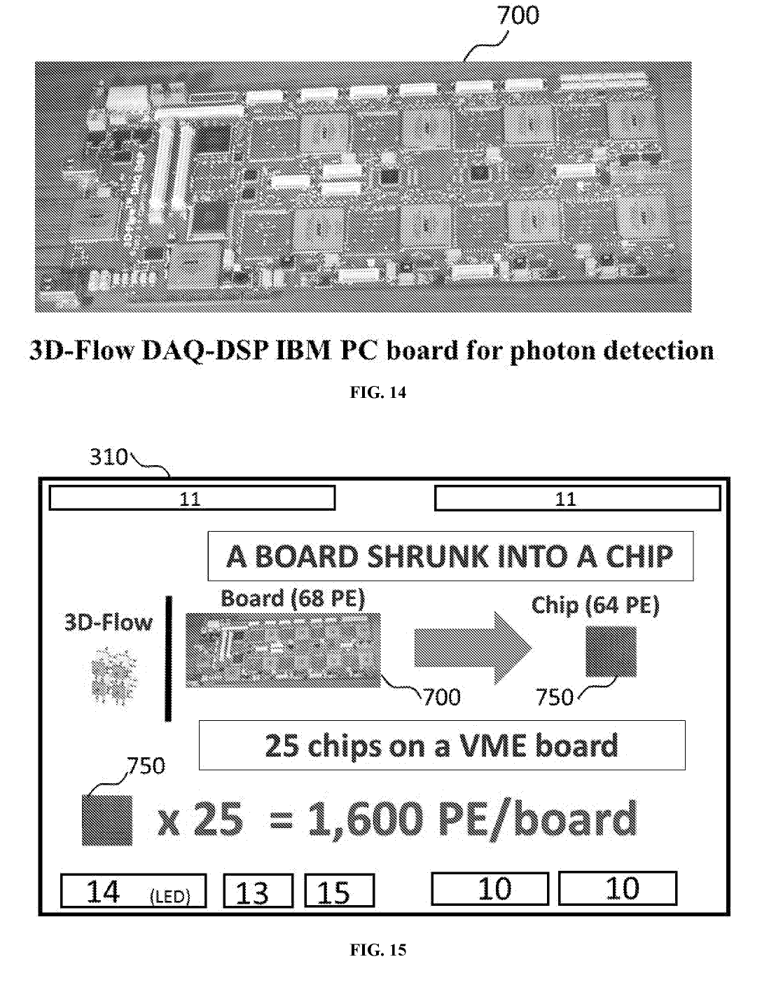

In Medical Imaging applications (see FIG. 6), the 3D-Flow, one of the basic inventions of the 3D-CBS (3-D Complete Body Screening), together with other inventions I conceived after the year 2000, offers a powerful, cost-effective very low radiation diagnostic tool capable of extracting all valuable information from radiation (radioisotope) associated with biological processes, and provides an unprecedented means to effectively detect anomalies such as cancer and many other diseases in those biological processes at an early curable stage. This has the potential to save millions of lives and significantly reduce Healthcare costs. If the current DOE HEP budget does not allow the funding of large generic R&D projects like the 64×3D-Flow chip, boards and systems, because our duty is to serve our leaders and together serve humanity, separate funding should be sought to implement innovations beneficial to humanity that are supported by calculations and scientific evidence in order not to miss again this opportunity to save money, lives, and to create a powerful tool to advance HEP research.

By using the 3D-Flow architecture in the 3D-CBS (3-D Complete Body Screening) to extract ALL valuable information from radiation by thoroughly analyzing all events' raw data (radiation) received directly from the detector, filtering all spurious events and carrying information regarding different tissue density in X-ray, CT and from the tracer (radioisotope) in the 3D-CBS or in PET, medical imaging will be greatly improved because the extracted characteristics of the radiation can optimize measurements of all parameters at the same time (spatial resolution, sensitivity, energy of the detected particle, etc.)

1. Saving Lives: The 3D-Flow System when Used in the 3D-CBS (3-D Complete Body Screening) for Medical Imaging Will have a Significant Impact on Cancer in Both Numbers and Healthcare Costs, Causing a Sharp Downturn in the Number of Cancer Deaths.

The most difficult task that I had to face for over a decade is to make people aware of the benefits to humanity of a Medical Imaging device capable of cost-effectively extracting ALL valuable information from radiation that could have already saved many lives and reduced healthcare costs. The people include political leaders, health care organizations, doctors, hospital administrators, cancer organizations, funding agencies, philanthropists, foundations supporting humanitarian causes, cultural groups and anyone who cares to defeat the most deadly and costly calamity, cancer, and who cares to advance health care with better diagnostic devices that give less radiation to the patient and provide more accurate information to doctors to helping them diagnose, prognoses and monitor treatment.

I believe I would befalling in my duty if I did not draw your attention to the adversities and obstacles that I have faced this past decade that have delayed implementation of my invention capable of extracting ALL valuable information from radiation, and the resulting burden to taxpayers this inaction has brought, not only monetarily but in avoidable deaths and suffering that will continue to occur unless immediate action is taken and the NRE (Non-Recurring Engineering) of the 3D-Flow and 3D-CBS projects are funded.

I hope that newspaper, radio, television and online media journalists as well as social networks feel a responsibility toward those who suffer and die needlessly and will reprint and disseminate this information and the link to this proposal so that everyone, including funding agencies handling taxpayer money or donations, will request a fair PUBLIC merit review that will reveal the scientific truth to benefit humanity.

Why is Extracting All Valuable Information from Radiation Important to Improve Early Cancer Detection?

Radiation is related to biological processes, therefore by accurately extracting all valuable information from radiation (on spatial resolution, time resolution, energy and sensitivity) it allows a reduction in the radiation dose to the patient, reduces costs and provides valuable information to doctors on anomalies in morphological changes and in biological processes, enabling improved diagnoses, prognoses and monitoring of the treatment of many diseases, while reducing healthcare costs.

How is All Valuable Information from Radiation Extracted?

By implementing a harmonious combination of several features ALL concurring to optimize the execution of Object Pattern Real-Time Recognition Algorithms (OPRA) on thousands of radiation data arriving in parallel from the detector at a very high speed. The 3D-Flow architecture is capable of executing uninterruptable complex algorithms for a time longer than the time between two consecutive input data sets by adding layers of 3D-Flow processors communicating through a bypass switch assuring zero dead-time. I then minimized the time required to exchange data between neighboring elements necessary for the execution of typical 3×3, 4×4, 5×5, . . . Object Pattern Real-Time Recognition Algorithms. The number of layers of 3D-Flow processors needed is calculated by dividing the time to execute an algorithm by the time interval between two consecutive input data and rounding the result up to the next integer. Because longer cables increase the algorithm's execution time (e.g. exchanging data between processors on different PCB boards connected through a 30 cm cable adds more than 1,000 picoseconds), this proposed system of 14,000×3D-Flow processors is confined to a 16 cm×16 cm×16 cm cube which keeps the number of layers of processors low and consequently lowers the power consumption of the system. The performance of this 3D-Flow system in recognizing objects by analyzing data arriving at a very high speed is far superior to any alternative system built with FPGA (Field Programmable Gate Array). This is because in FPGA a lot of silicon area with electronic circuits which are not optimized for OPRA consume power, requiring a larger system and longer cables, thus never able to achieve the performance and lower cost of the 3D-Flow.

How Much does the 3D-Flow OPRA System Cost to Extract all Valuable Information from Radiation to Make a Revolution in an Effective Early Detection that could Save Many Lives and Reduce Healthcare Costs?

A 2,304 channels 3D-Flow for the 3D-CBS device for early cancer detection is very competitive with other instrumentation such as oscilloscopes and Logic State Analyzers. As you can see from Table 1, Table 2 and Table 3, the cost per channel supported by the quotes from reputable companies reported in the budget justification is much lower than oscilloscopes and Logic State Analyzers.

C. Justification of the Proposed Project

The proposed project is justified from the need to create a powerful tool with the capability to extract all valuable information from radiation more specifically to execute experimenters' programmable complex Object Pattern Real-Time Recognition Algorithm at the Level-1 trigger with neighboring data exchange while sustaining an input data rate of 80 MHz from 8,000 channels, each receiving a 16-bit word (or 40 MHz with a 32-bit word, or 20 MHz with 64-bit word) with zero dead-time. This proposed project is also justified by the need to develop a similar powerful tool capable to extract all valuable information from radiation in the application of medical imaging to reduce the radiation dose to the patient, to enable an effective early cancer detection and reduce healthcare cost.

An aggressive schedule following a prompt funding could target the installation of the first prototype in one of the large experiments at CERN before Jan. 31, 2018. In the event CMS and Atlas management will not agree to adopt the 3D-Flow trigger, arrangements should be made to derive the 8,192 trigger signals from the two experiments and send them in parallel to the ATCA-PRAI crate (Patch Panel Regrouping Associates Ideas, see FIG. 4) that will transfer the trigger data at 1.3 TB/sec to the 3D-Flow system consisting of 43,008×3D-Flow processors to work in parallel to the official trigger system.

In the event it is not possible to split the signals of trigger raw data and send one to the official Level-1 Trigger and the other to the 3D-Flow system it will still be fine to receive a copy of the data buffered from the official Level-1 trigger. Even if these data will arrive to the 3D-Flow with a latency, I am confident that the 3D-Flow system can process the raw data and generate a Global Level-1 trigger decision within the time of a few microseconds allocated for Level-1 Trigger decision.

No matter how many superlative adjective one can use to describe success, experimental results tell the truth that only 40 Higgs boson-like events found on Jul. 4, 2012 out of 100,000 generated by LHC is a failure of the Level-1 Trigger. Moreover, these 40 Higgs boson-like events were not found by the Level-1 trigger but from analysis of trillions of data recorded casually.

These facts & data are confirmed by CERN slides and admitted in CERN-Atlas and CERN-CMS experiments upgrade official documents and it is also stated by the presenters of the trigger of the large experiment at CERN at the IEEE conference.

D. Excerpts of My Answer to the Director of DOE-HEP's Request Dated Aug. 4, 2015

The following is an excerpt from my 20 page answer to Dr. Siegrest's request to provide “the most important ingredient” supporting a “compelling case for you [me] to visit” that I sent on Sep. 20, 2015.

Thank you Jim for answering my email and inviting me to submit a “compelling case for you [me] to visit.” My highly compelling case is summarized in the following paragraph, presented at many conferences, published in articles, and sent in emails, as in the material related to my last abstract, slides and presentation that I sent to you in my email on Jul. 16, 2015:

“The innovative 3D-Flow parallel-processing architecture, and fault-tolerant system, recognized valuable by major, public, scientific reviews, proven feasible and functional in hardware, breaks the speed barrier in real-time applications such as in High Energy Physics (HEP) and Medical Imaging. It is flexible, scalable, programmable, modular, technology-independent meaning it can migrate to the most advanced and cost-effective technology, improving since 2003 its speed by over 13 times, lowering power consumption to 1/10 and lowering the cost to less than 1/200. In addition the 3D-Flow innovative architecture offers system performance at a speed higher than the technology performance. It offers the most cost-effective and powerful tool to researchers in High Energy Physics to discover new particles and can save many lives with a cost-effective early cancer detection tool In HEP applications, the 3D-Flow architecture, is capable of executing experimenters' desired programmable complex Object Pattern Recognition Level-1 Trigger (OPRT) algorithms, while sustaining an input data rate of over 80 million events per second from over a billion collisions per second, with zero dead-time, at a lower cost (compared to current approaches) per each good event captured and fully meeting 1994 LHC experiment requirements, 2012, today and future requirements for a higher LHC luminosity. In Medical Imaging applications the 3D-Flow as one of the basic inventions of the 3D-CBS (3-D Complete Body Screening), offers a powerful, cost-effective diagnostic tool capable of extracting all valuable information from radiation (radioisotope) associated with biological processes and provides an unprecedented means to effectively detect anomalies in biological processes such as cancer and many others diseases at an early curable stage, administering a very low radiation dose and a very low examination cost. This has the potential to save millions of lives and significantly reduce Healthcare costs. This is not propaganda or advertisement. Each statement above is supported by the 3D-Flow feasibility and functionality proven in hardware, by calculations, by correct equations, by scientific evidence. The speed, silicon die size, cost, power consumption from nW/MHz/gate to the power consumption of the 3D-Flow chip, to the power consumption of the VME 9U or 6U electronic boards, to the kW consumption in a crate, for a specific technology are confirmed by the parameters specified by world's major companies, achievable with their technology and manufacturing process to make Integrated Circuits, electronic boards and crates. If the current DOE HEP budget does not allow the funding of a generic R&D project like the 64×3D-Flow chip, boards and crate, because our duty is to serve our leaders and together serve humanity, it will be important for everyone to inform them to provide separate funding and the media inform the public about innovations beneficial to humanity that are supported by calculation and scientific evidence in order not to continue to miss this opportunity to save money, lives, and to create a powerful tool to advance HEP research”

1. The Entire Level-1 3D-Flow Readout-Processing Boards and Global Trigger for 8,192 Trigger Towers can be Implemented in One of the Following Platforms:
1. Eight 1024 channels 3D-Flow boards 9U VME, 366 mm×400 mm with the capability to execute up to 30 Object Pattern Real-Time Recognition Algorithm 3D-Flow steps @ 40 MHz 32-bit input data rate or 15 steps at @ 80 MHz 16-bit input data rate and one 3D-Flow Global Trigger board. The boards consist of eight 9U VME Readout-Processing boards with partial pyramid for channel reduction and one 9U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   OR
2. Sixteen 512-channels 3D-Flow boards 9U VME, 366 mm×400 mm with the capability to execute up to 70 Object Pattern Real-Time Recognition Algorithm 3D-Flow steps @ 40 MHz 32-bit input data rate or 35 steps at @ 80 MHz 16-bit input data rate and one 3D-Flow Global Trigger board. The boards consist of sixteen 9U VME Readout-Processing boards with partial pyramid for channel reduction and one 9U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   OR
3. Thirty-two 256 channels 3D-Flow boards 6U VME, 233 mm×160 mm with the capability to execute up to 50 Object Pattern Real-Time Recognition Algorithm 3D-Flow steps @ 40 MHz 32-bit input data rate or 25 steps at @ 80 MHz 16-bit input data rate and one 3D-Flow Global Trigger board.
   The boards consist of thirty-two 6U VME Readout-Processing boards with partial pyramid for channel reduction and one 6U VME Global Trigger board with the final section of the pyramid, the calculation of the global quantities that are provided to the Global Trigger for the final Level-1 trigger decision. The global 3D-Flow system can sustain 80 million events per second from over a billion collisions per second, with zero dead-time. Each of the 8000 input channels receive 16-bit data every 12.5 ns. Each 3D-Flow program step has the capability to perform up to 26 operations of addition, subtraction, compare with multiple values, etc. This provides a complete, thorough Object Pattern Recognition capability using information from multiple detectors (calorimeter, tracking, etc.).
   Many 9U Crates, Current Level-1 Trigger OR Two 6U Crates with 32×3D-Flow 256 Ch. Boards.

2. Supporting Claims for Programmability, Sustaining Very High Input Data Rate at a Low Cast

The 3D-Flow is able to extract all valuable information from radiation to discover new particles and save billions of dollars in physics research with a box of electronics containing 50,000×3D-Flow processors costing less than $1 each.

The study, verification of feasibility and functionality with FPGA and with the silicon foundries that can produce the 3D-Flow processor at less than $1 per processor and the 3D-Flow system in one box of electronics has been done.

The words “ . . . capable of executing experimenters' desired programmable complex Object Pattern Recognition Level-1 Trigger (OPRT) algorithms” were recognized in several letters by top experts in the field when praising my innovative 3D-Flow architecture. This is reported in the attachment, which includes the letter dated Nov. 5, 1993 from Andy Lankford, former Deputy Spokesman of CERN-LHC-Atlas experiment, the letter dated Feb. 11, 1993 from Livio Mapelli, leader of the same experiment at CERN, the letter dated Feb. 27, 1995 from Joel Butler, head of the computing division at FERMILab, and in many other letters. Lankford wrote: “ . . . a technique to perform fast, programmable triggers”, while Mapelli wrote: “ . . . higher flexibility, coming from the programmability of algorithms and not only of parameters . . . ” and Butler, one of the organizers of the major FERMILab review of my 3D-Flow invention wrote: “the 3D-Flow project is the only detailed study demonstrating the feasibility of executing several level-1 trigger algorithms of different experiments.” These are clear statements recognizing the capability (and later confirmed feasible and functional in hardware) of the 3D-Flow system to execute Object Pattern Recognition Trigger (OPRT) algorithms not possible at level-1 trigger before my invention. I invite anyone to provide a reference to any system developed during the past 23 years which has some level of programmability and compare the system's flexibility, performance and cost-effectiveness to the 3D-Flow system. On page 13 of the 2013 CMS and Atlas Level-1 Trigger upgrade official document, they admit their current Level-1 Trigger does not have OPRT capability and will be trashed. They plan to implement OPRT over the next 15 years making use of FPGA which is hundreds of times less efficient, less performant and more costly than my 3D-Flow solution.

The words: “while sustaining an input data rate of over 80 million events per second from over a billion collisions per second . . . ” As you will see from the evidence presented at the end of this document, current technology allows me to build a 3D-Flow system with the capability to surpass, at zero dead-time, the requirements of 80 MHz that is planned for the LHC to run a few years from now. The 3D-Flow architecture would have also surpassed the 20 MHz requirements of the LHC for most of the runs in 2011-2012 with the 3D-Flow FPGA boards running at 31 MHz, and the 3D-Flow version in 350 nm standard cell technology running at 61 MHz compiled with Synopsys tools in 1996 (but without funding it was never built in silicon) would have fully satisfied with zero-dead-time the requirements of the few runs of the LHC in late 2012 at 40 MHz bunch crossings. All the 3D-Flow versions in FPGA or Standard cell 350 nm technology had the capability of analyzing ALL billion collisions per second.

I invite anyone to provide a reference to any system developed during the past 23 years which can cope with even 40 million events per second or one which does not have limitations in handling a billion collisions per second. This is no problem for the 3D-Flow architecture which can satisfy these requirements even with technology of 25 years ago when it first was announced.

The words: “with zero dead-time . . . ” The 3D-Flow architecture clearly described in one page and simulated in an analogy by high school students show that data from all bunch crossings, with zero dead-time are thoroughly analyzed, even if the technology is slower than the input data rate. Even nowadays there is not a Level-1 trigger system having zero dead-time, and even those designed and built for the upgrades of the Atlas and CMS experiments at LHC for the next 15 years do not provide zero dead-time. The presenters of Atlas and CMS papers at the Trigger Session of the 2013 IEEE-NSS conference in Seoul, South Korea, even admitted that when Level-1 Trigger fires, the system is dead for 3 to 4 bunch crossings. What if during those bunch crossings the rare Higgs boson-like event which occurs only once every 10 billion events is missed? It was instead recognized in 1992 that my 3D-Flow invention provides the zero dead-time system capability. At the NSS-N5-4 presentation at the 2013 IEEE-NSS-MIC-RTSD conference, it was stated that the Atlas L1 Trigger be improved to cope with higher rates (slide 17). Slide 16 states that “no change to the detector is needed. Full replacement of front-end and back-end electronics” was needed instead. This was not because of aging radiation limits. The limitation of the current Atlas trigger was admitted.

The words: “ . . . at a lower cost . . . ,” the 64×3D-Flow processors on a chip cost less than 1/200 the price of the 64×3D-Flow processors on the board I built in 2003 and displayed later in this document.

Each group of words in the long paragraph needs to be addressed in depth. However, when it comes to the words referring to Medical Imaging applications the urgency to address all words are even more important because it will prevent more years of lives from being lost which could have been saved with an effective early cancer detection that I made available more than a decade ago.

The words: “ . . . offers a powerful, cost-effective diagnostic tool capable of extracting all valuable information from radiation associated to biological processes . . . ” is not up to the competence of doctors but the competence of scientists expert in particle detection, because for a doctor in medicine it would be difficult to grasp the magnitude of the benefit of the invention providing the capability to extract more efficiently information from radiation. It is competence of scientists expert in particle detection understand the advantages of the right column “3D-CBS with innovations” with respect to the column to its left “Current PET & PET/CT” of FIG. 1 on page 2 of the article “The 3-D Complete Body Screening (3D-CBS) Features and Implementation” compares the advantages of the right column “3D-CBS with innovations” to the left column “Current PET & PET/CT”. It would take a scientist expert in particle detection to fully understand these advantages and the benefits of executing a 3×3 matrix algorithm with the 3D-CBS device instead of a 2×2 anger logic algorithm calculated on current PET devices. The 3×3 can provide more accurate information on the photon's total energy, rejecting more efficiently scatter events and capturing more good 511 keV pairs of photons. It would also allow an increase in spatial resolution interpolating the location of the incident photon in the crystal with information from detector elements to the left and to the right (up and down for “y” coordinate calculation) of the position where the photon hit the detector. The capability to execute complex algorithms as shown in section “r” (bottom right of FIG. 1) allows to extract more information from economical crystals, to improve photon arrival time from economical crystals with long decay time, thus lowering the cost of the 3D-CBS device with a detector having a FOV ten times longer than current PET but costing only twice as much and capable of capturing hundreds of times more the number of signals from the tumor markers (photons emitted from the radioisotope). This makes the examination shorter, increases throughput which further lowers the examination cost, reduces the radiation to the patient and enables an effective early detection.

If improving particle detection is key to improving early detection of cancer and other diseases that can save many lives, then it is essential to address in depth what is improving particle detection to provide benefits to humanity in saving lives and reducing Healthcare costs.

Examples of Object Pattern Recognition Trigger (OPRT) algorithms coded in “steps” that can be executed in a single cycle by the 3D-Flow processors. These powerful “steps” (or 3D-Flow instructions) are key tools in giving experimenters the power to nail down their new particle and detect it among an increasing background noise that occurs with an increased luminosity of LHC. These instructions/steps can also resolve pileup by detecting a slope change in the signal. Many experts in the field including Nick Ellis, Andy Lankford, Livio Mapelli and Joel Butler understood and recognized over 20 years ago the power of the 3D-Flow architecture that can execute several of these programmable “steps”. Several put it on writing like Joel Butler who wrote: “the 3D-Flow project is the only detailed study demonstrating the feasibility of executing several level-1 trigger algorithms of different experiments.” For this reason I also highlighted in the specifications of the “3D-Flow decision box, or Trigger” the number of “steps” different configurations of 8 boards 9U, 16 boards 9U or 32 boards 6U can perform.

E. Requirements of the Level-1 Triggers of High Energy Physics Experiments

The specifications and requirements for the Level-1 Trigger of the largest, billion dollars experiments at LHC consist of 8,000 trigger towers (or channels) which were each receiving up to 10-bit information every 50 ns (20 MHz) until 2012, then 25 ns (40 MHz) in the current run with the prospect of up to 12.5 ns bunch crossings (80 MHz) in future upgrades.

Current requirements of the Level-1 Trigger for LHC experiments:
8000 channels or Trigger Towers with a detector granularity of Δη×Δφ=0.1×0.1
8 to 10-bit information of the E_{T} from the 8000 Trigger Towers in the Hadronic, Electromagnetic, and HF calorimeters every 25 ns
40 MHz bunch crossing
Capable of executing simple algorithms with limited programmability and performing simple operations that do not support a thorough object pattern recognition

F. The 3D-Flow System Exceeds all Current Requirements of the Level-1 Trigger for LHC Experiments but Because it is Technology-Independent, it can Increase its Performance as Needed to Satisfy Future Upgrade Requirements, Increasing the Triggers' Power to Discover New Particles.
8000 channels or Trigger Towers with a detector granularity of Δη×Δφ=0.1×0.1
16-bit information of the E_{T} from the 8000 Trigger Towers in the Hadronic, Electromagnetic, HF calorimeters, and from tracking detectors and other detectors with a detector granularity of Δη×Δφ=0.1×0.1, that can provide useful information for Level-1 Trigger every 12.5 ns
80 MHz bunch crossing
Capable of executing complex algorithms with full programmability from 30 to 70 steps @ 40 MHz input data rate or 15 to 35 steps @ 80 MHz input data rate. Each step with the capability to perform up to 26 operations such as addition, subtraction, comparison with multiple values, etc. This provides a thorough object pattern recognition capability using information from multiple detectors (calorimeter, tracking, etc.).

If necessary, it can handle a higher input data rate or more information at each of the 8000 input channels exceeding the 16-bit every 12.5 ns. For example, if additional information from tracking or other sub-detectors could be useful at the Level-1 Trigger decision, the 3D-Flow system can be designed to handle 24-bit or 32-bit words received from each of the 8000 channels every 12.5 ns.

The Staggering Cost-Performance Advantages of the 3D-Flow System Compared to Other Trigger Approaches is Because:

The tested functionality of the 64×3D-Flow processors housed in 16 FPGA chips, each with 4×3D-Flow processors in this 3D-Flow IBM PC board can now be implemented in a 35 mm×35 mm chip with 64×3D-Flow processors with staggering performance increase and lower cost.

3D-Flow DAQ-DSP IBM PC modular board with 68×3D-Flow processors, 2,211 components, over 20,000 contact pins connected through only 8 layers printed circuit board for signals and 6 layers for power and ground. The 68×3D-Flow processors are housed in 17 large FPGA from Altera, each with 4×3D-Flow processors.

The board and the system worked at the first prototype. The signals transmitted over LVDS connections provide stability and noise immunity to the system even when several signals switch at the same time.

The board designed and built by Crosetto in 2003 had unprecedented performance in guaranteeing the clock distribution to the pin of every component in a system made of several of these modular boards in different crates with a maximum difference between any two pins of 40 picoseconds.

THIS BOARD CAN NOW BE REPLACED BY A CHIP WITH 64×3D-FLOW PROCESSORS
OVER 13 TIMES FASTER
CONSUMING LESS THAN 1/10 THE POWER
COSTING LESS THAN 1/200

64×3D-Flow Processors IC, 35 mm×35 mm

To migrate the technology-independent 3D-Flow design of 4× processors per chip to either 16×, 64×, or 256×, optimized for the best cost-performance, a study was conducted for the 180 nm, 130 nm, 90 nm, 65 nm and 40 ns for the generic process, the LP (low power) and for the ULP (Ultra Low Power) technology processes. The 28 nm leading edge or 14 nm bleeding edge technologies were not considered because of the high cost and low volume that would not justify the NRE cost. Another reason is that the 3D-Flow architecture offers performance breaking the speed barrier of the technology used, therefore optimization is focused on price-performance and not on absolute maximum performance.

The major IC design houses and silicon foundry in the world were contacted to ensure all options were considered. Considering the speed, power consumption, cost per 3D-Flow processors, cost and performance of IP such as LVDS, PLL, USB, etc. for different technologies, the optimization was achieved in selecting 64× processors per chip with 112 pins carrying signals to/from each group of processors facing the North, East, West and South (NEWS) sides, 256 pins for signals sent out from the Bottom port, 256 pins receiving signals from the Top port and 26 control signals.

The risk involved in this migration from 4×3D-Flow processors per chip to 64× is minimal or close to zero because its functionality has been already tested with simulators and in hardware on the 4×3D-Flow processors per chip and 68×3D-Flow processors on two boards. Accurate calculation of power consumption from nW/MHz/gate to the power consumption of the chip, of the power consumption of the VME 9U or 6U electronic boards, to the kWatt consumption in a crate were performed as well as the number of signals and speed that needs to be transmitted from chip-to-chip, from board-to-board and from crate-to-crate.

The entire 3D-Flow system for the 3D-CBS extracting all valuable information from radiation (radioisotope) to provide an effective Early Cancer Detection at a very low radiation dose and examination cost requires 2,304 channels that can fit in one 6U VME crate Nine 256 channels 3D-Flow boards 6U VME, 233 mm×160 mm with the capability to execute up to 100 Object Pattern Real-Time Recognition Algorithm 3D-Flow steps @ 20 MHz 64-bit input data rate and one 3D-Flow Coincidence Detection board. The system consists of nine 6U VME Readout-Processing boards with partial pyramid for channel reduction and one 6U VME Coincidence Detection board with the final section of the pyramid to further reduce the number of channels and the detection of 511 keV pairs of photons in time coincidence.

The global 3D-Flow system can sustain the highest possible data rate which is limited by the speed of the crystal detector capable of capturing millions of pairs of photons in time coincidence per second, with zero dead time. Each of the 2,304 input channels receive up to 64-bit data every 50 ns.

Each 3D-Flow program step has the capability to perform up to 26 operations, such as addition, subtraction, comparisons of multiple values, etc. This provides a complete, thorough Object Pattern Recognition capability using information from multiple sensors (SiPM front and back, or PMT).

One 6U VME Crate with 9×3D-Flow 256 Ch. Boards and 1×3D-Flow Coincidence board 3D-Flow photon detection algorithm max 100 steps @) 20 MHz 64-bit input data rate

The 3D-CBS is a safe revolutionary device for Early Detection and Prognosis of cancer, and to screen for cancer on asymptomatic people when it is most curable

It can also detect other diseases presenting abnormal biological processes

Experimental data demonstrates that Early Cancer Detection saves over 50% of lives

In summary, my breakthrough invention is a 64×3D-Flow processors chip that is over 13 times faster, consumes less than 1/10 and costs less than 1/200 the previous 64×3D-Flow processors version which was already proven feasible and functional in a board.

These staggering improvements mean that several 9U VME crates of the Level-1 Trigger at the largest HEP experiments can be replaced with a single 9U VME crate with either 9 boards or 17 boards, increasing the power of the tools to discover new particles and provide benefits in many other field such as in Medical Imaging to save lives and reduce healthcare costs.

I would welcome another open international review like the one at FERMILab held on Dec. 14, 1993, when for the entire day I answered all questions satisfactorily regarding my 3D-Flow and supported my calculations that ultimately were proven correct by implementing the two 6×3D-Flow processors boards in FPGA. This would save the development of many trigger crates of electronics planned for the upgrade of CMS and Atlas experiments costing millions of dollars.

To migrate the technology-independent 3D-Flow design of 4× processors per chip to either 16×, 64×, or 256×, optimized for the best cost-performance, we conducted a study for the 180 nm, 130 nm, 90 nm, 65 nm and 40 ns for the generic process, the LP (low power) and for the ULP (Ultra Low Power) technology processes.

The major IC design houses and silicon foundry in the world were contacted to ensure all options were considered. The risk involved in this migration from 4×3D-Flow processors per chip to 64× is minimal or close to zero because its functionality has been already tested with simulators and in hardware on the 4×3D-Flow processors per chip and on two modular boards, each with 68×3D-Flow processors. Accurate calculation of power consumption from nW/MHz/gate to the power consumption of the chip, to the power consumption of the VME 9U or 6U electronic boards, to the kWatt consumption in a crate were performed as well as the number of signals and speed that needs to be transmitted from chip-to-chip, from board-to-board and from crate-to-crate.

G. Examples of Implementations of the 3D-Flow Multiprocessor System on a VME Form Factor

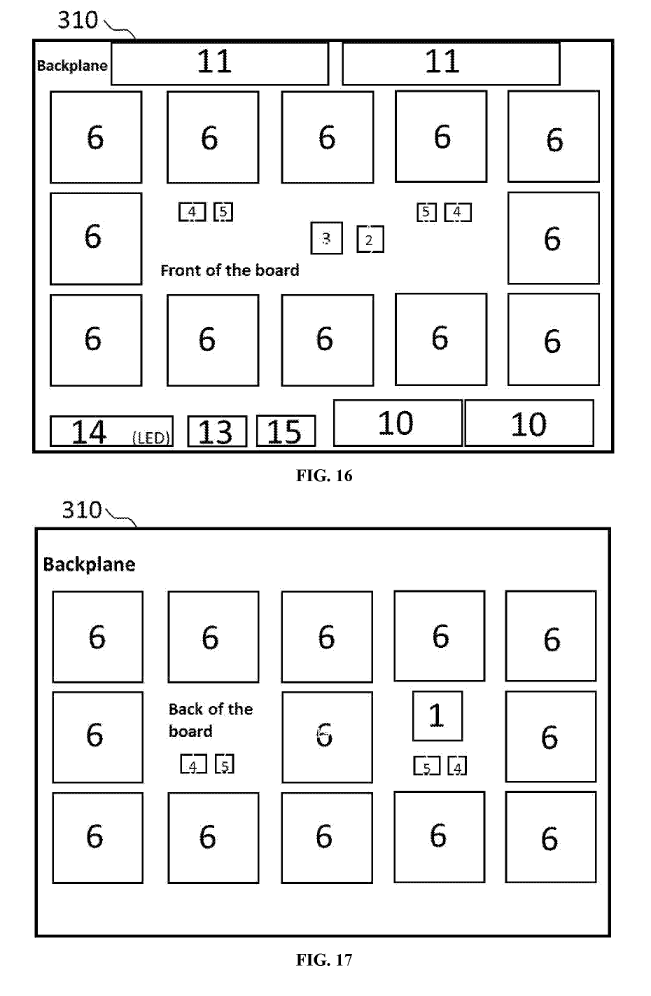

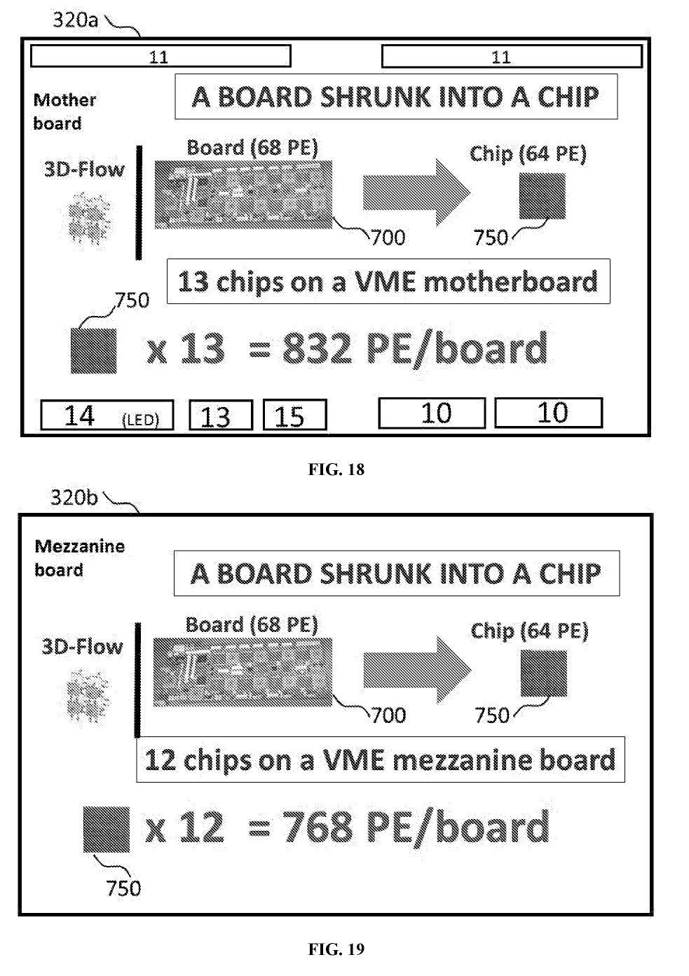

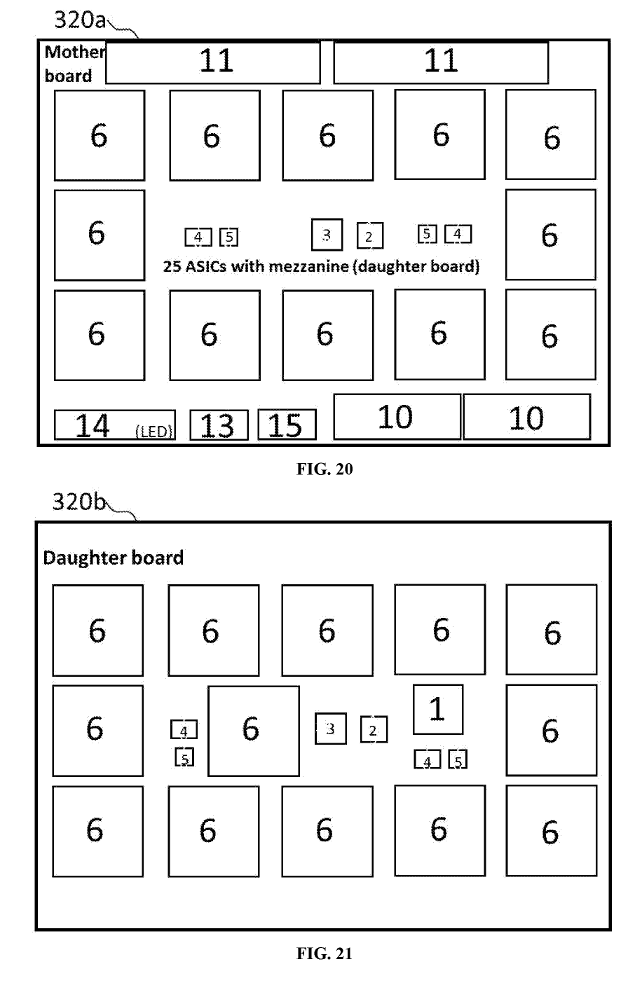

See FIG. 15, FIG. 16, FIG. 17, FIG. 18, FIG. 19, FIG. 20, FIG. 21.

Following is an example of specifications, a drawing of the logical block diagram, the layout of the components on the board, and a template to prepare the quote for the 3D-Flow VME form factor electronic board.

The template that I prepared for the different companies that were interested in bidding on this project were the following as reported in the Excel spreadsheet of Table 4

TABLE 4
Excel spreadsheet provided to the companies interested in
bidding for the 3D-Flow VME form factor electronic board.

| Item | SUMMARY 6U-VME size module | Item | Notes | | |
|---|---|---|---|---|---|
| # ID | DESIGN + NRE + PROTOTYPE | Category | | Volume for | Volume for |
| Item | SMALL/LARGE PRODUCTION | Item | | 20 modules | 20 modules |
| # ID | Description of the material | Category | Qty | (unit cost) | (module cost) |
| 22 | ID = 1, 324 pins, 1 mm pitch, dimensions 19 × | FPGA | 1 | $50.0000 | $50.0000 |
| | 19 mm, or equivalent | | | | |
| 23 | ID = 2, 32-Lead, LQFP, dimensions 10 mm × | Delay-line | 1 | $15.0000 | $15.0000 |
| | 10 mm, power 0.2 W | | | | |
| 24 | ID = 3, 64-Lead, LQFP, dimensions 12 mm × | Clock_Driver | 1 | $25.0000 | $25.0000 |
| | 12 mm, power 1 W | | | | |
| 25 | ID = 4, 20 pin, dimensions 10 mm × 7 mm, or | DCDC-REG | 4 | $15.0000 | $60.0000 |
| | Empirion, or equivalent | | | | |
| 26 | ID = 5, 2-pins, dimensions 7 mm × 7 mm, | Inductance | 4 | $5.0000 | $20.0000 |
| | power 0.1 W | | | | |
| 27 | ID = 6, 1681-pins BGA, dimensions 35 mm × | ASIC | 25 | $0.0000 | $0.0000 |
| | 35 mm, power 4.3 W | | | | |
| 28 | ID = 7, 2-pins, resistors 100 ohm for LVDS | Resistors | 4000 | $0.0040 | $16.0000 |
| 30 | ID = 8, 2-pins, capacitors, small | capacitors | 800 | $0.0800 | $64.0000 |
| 31 | | | | | $0.0000 |
| 32 | ID = 10, right angle connectors carrying 131 | Connectors | 2 | $30.0000 | $60.0000 |
| | LVDS front of the board SEAM-30-01-S-10- | | | | |
| | 2-RA-K-TR, or equivalent | | | | |
| 33 | ID = 11, connectors carrying 700 signals total, | Connectors | 2 | $4.0000 | $8.0000 |
| | rear of the board | | | | |
| 34 | ID = 12, Heat dissipation metal bars | Heat-bars | 4 | $20.0000 | $80.0000 |
| | ID = 13, output connector for 24 LVDS + 8 | Connectors | 1 | $2.0000 | $2.0000 |
| | single-ended signals | | | | |
| | ID = 14, two groups of 8 LED | LED | 1 | $1.0000 | $1.0000 |
| | ID = 15, connector for test points on front | Connectors | 1 | $2.0000 | $2.0000 |
| | panel (32 signals) | | | | |
| | ID = 16, Large capacitors | Capacitors | 50 | $0.5000 | $25.0000 |
| | ID = 17, Miscellaneous | Misc | 1 | $25.0000 | $25.0000 |
| 36 | Purchasing cost 7% of component cost | | | $13.6209 | $31.7100 |
| 37 | ID = 18, PCB cost, dimensions 233 mm × 160 | PCB | 1 | $320.0000 | $320.0000 |
| | mm | | | | |
| 38 | item . . . ?? (specify ??) | | | | |

| | | |
|---|---|---|
| 39 | TOTAL BOM Cost per Module | $804.7100 |

H. Examples of Implementations of the 3D-Flow Multiprocessor System on a VXI Form Factor

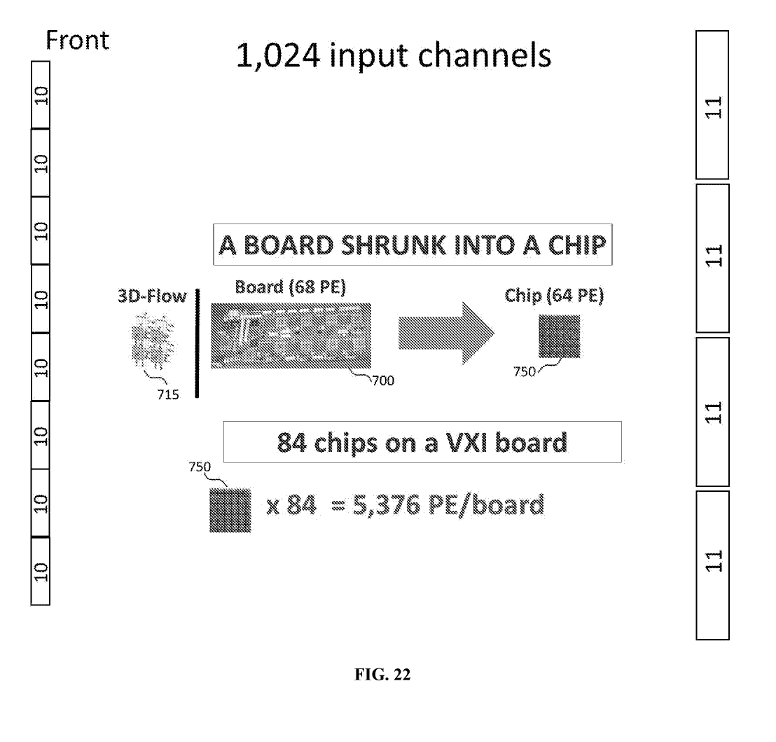

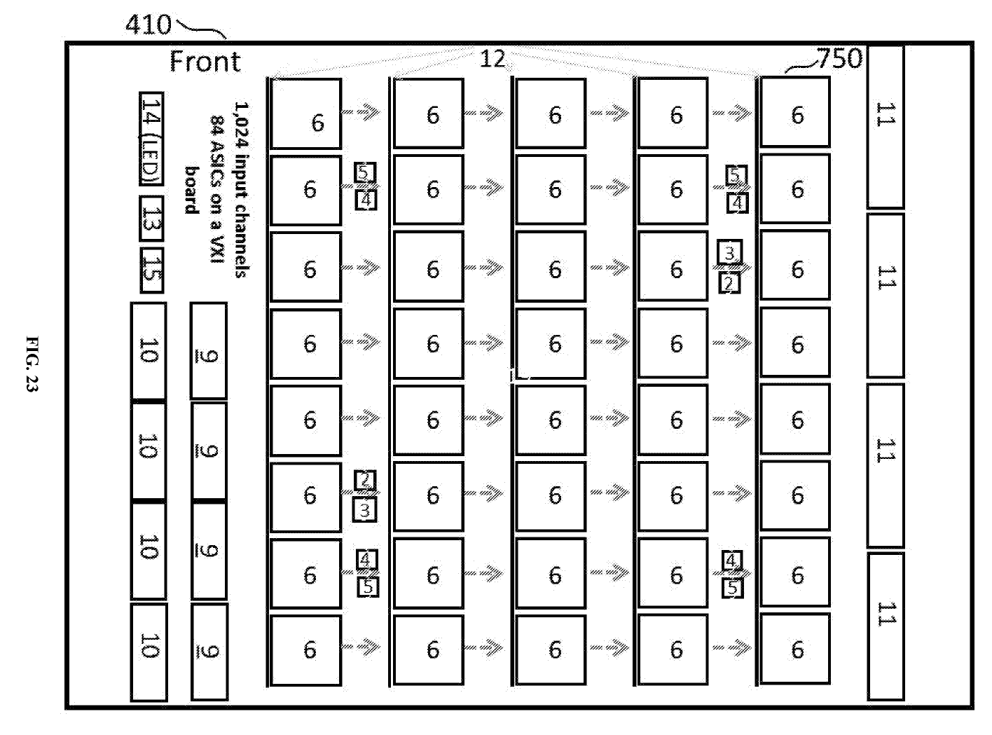

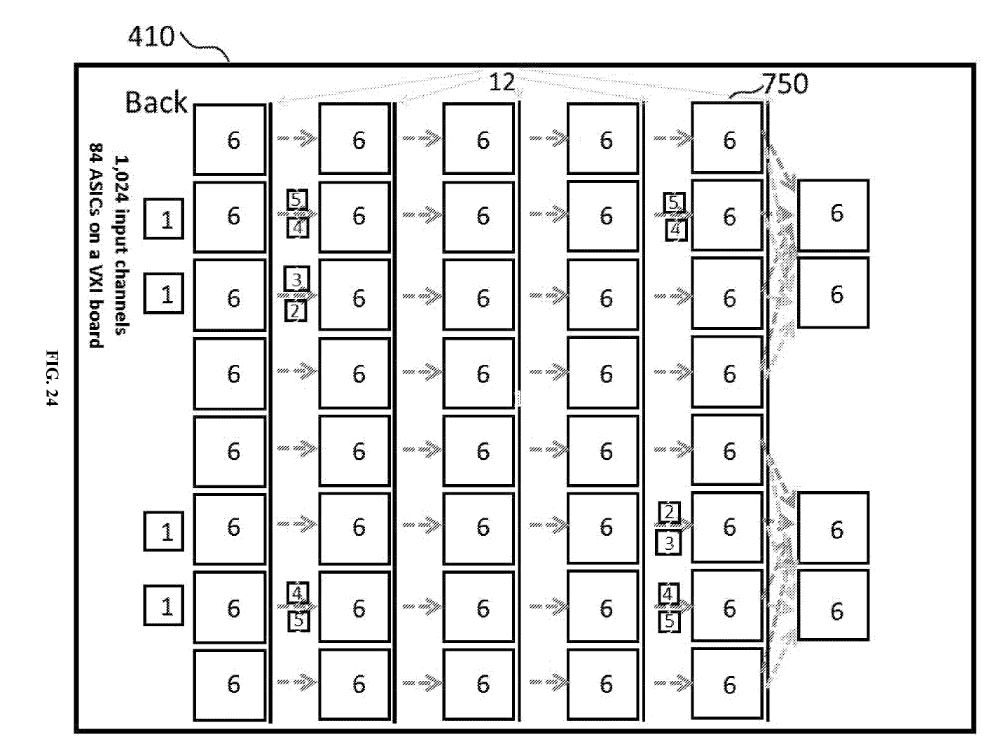

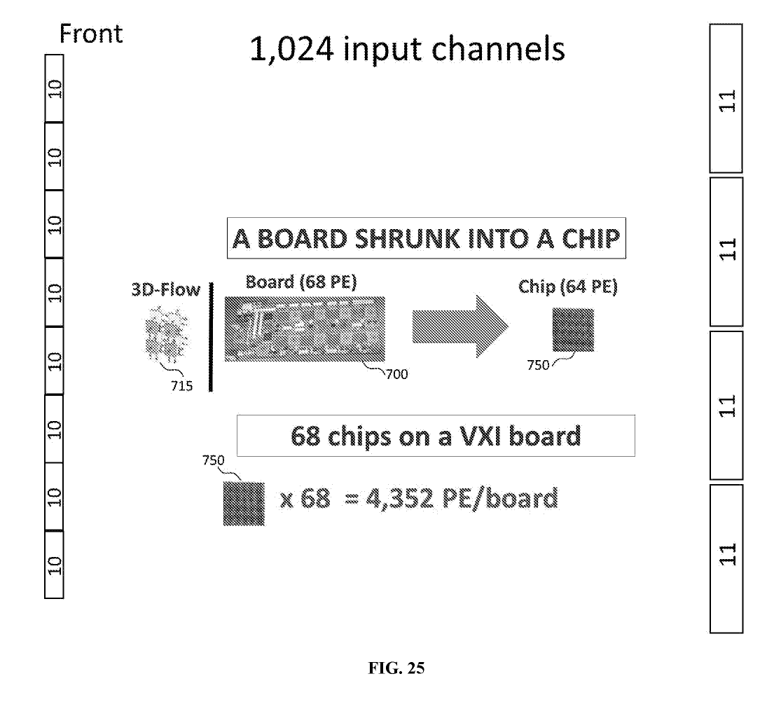

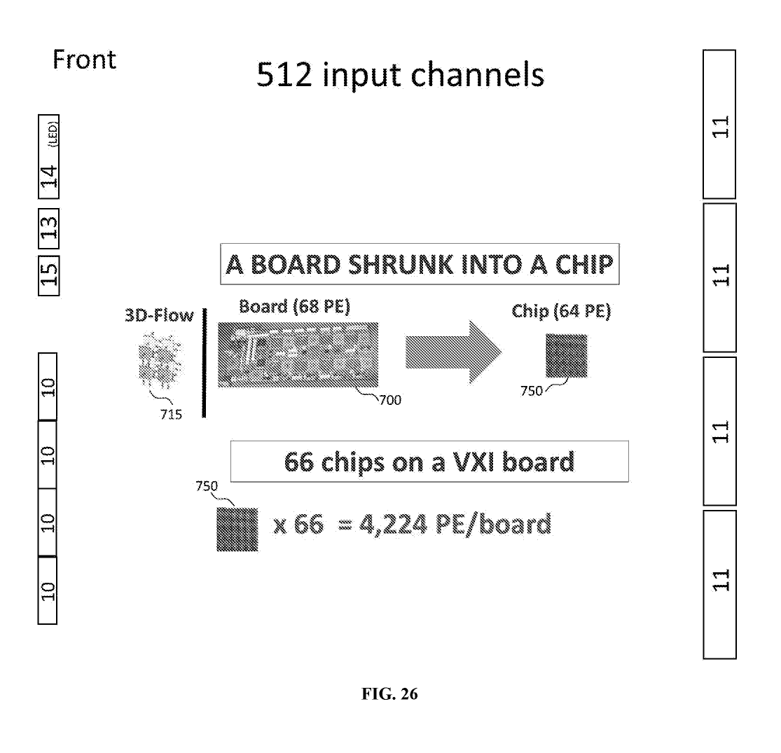

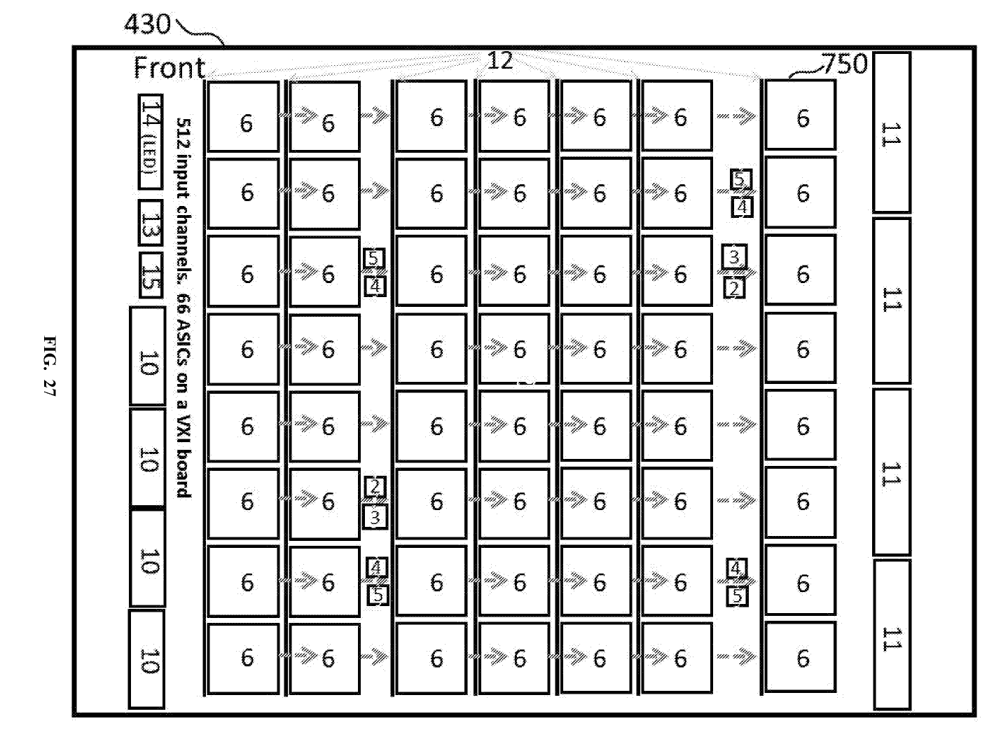

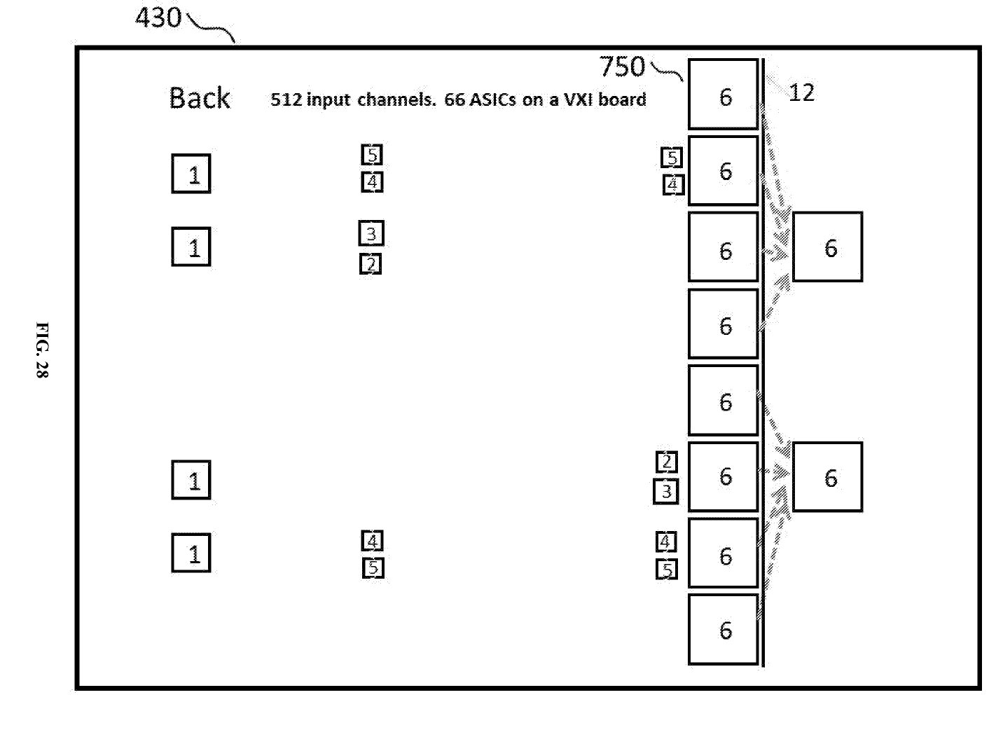

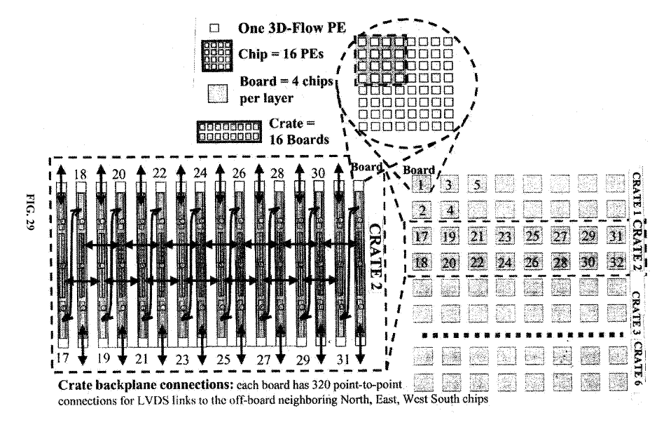

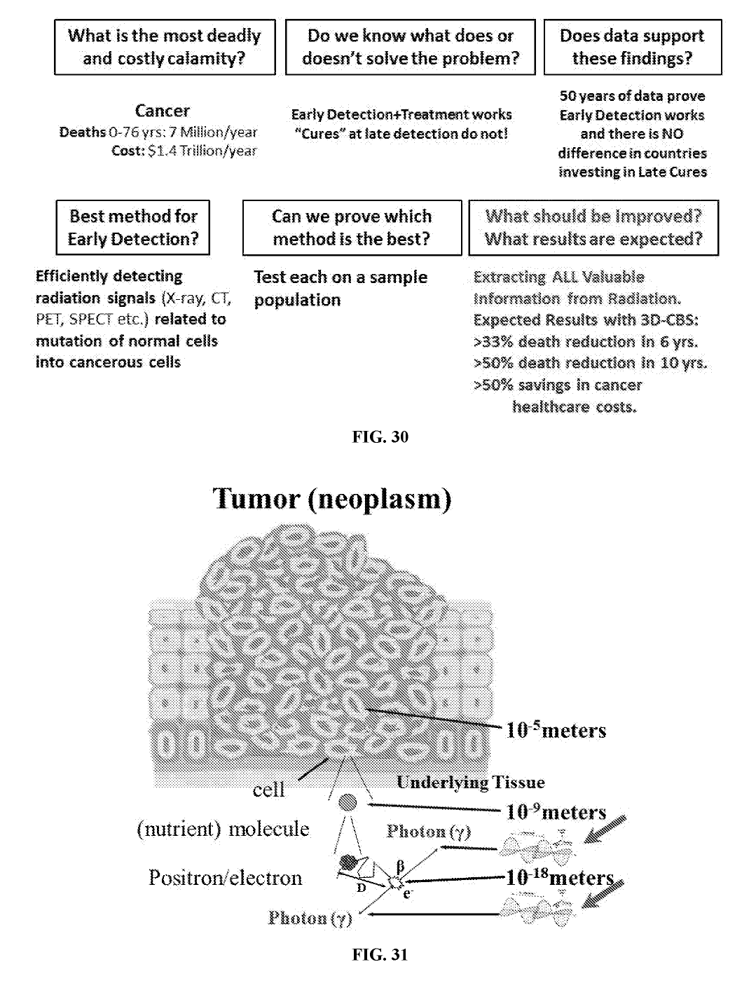

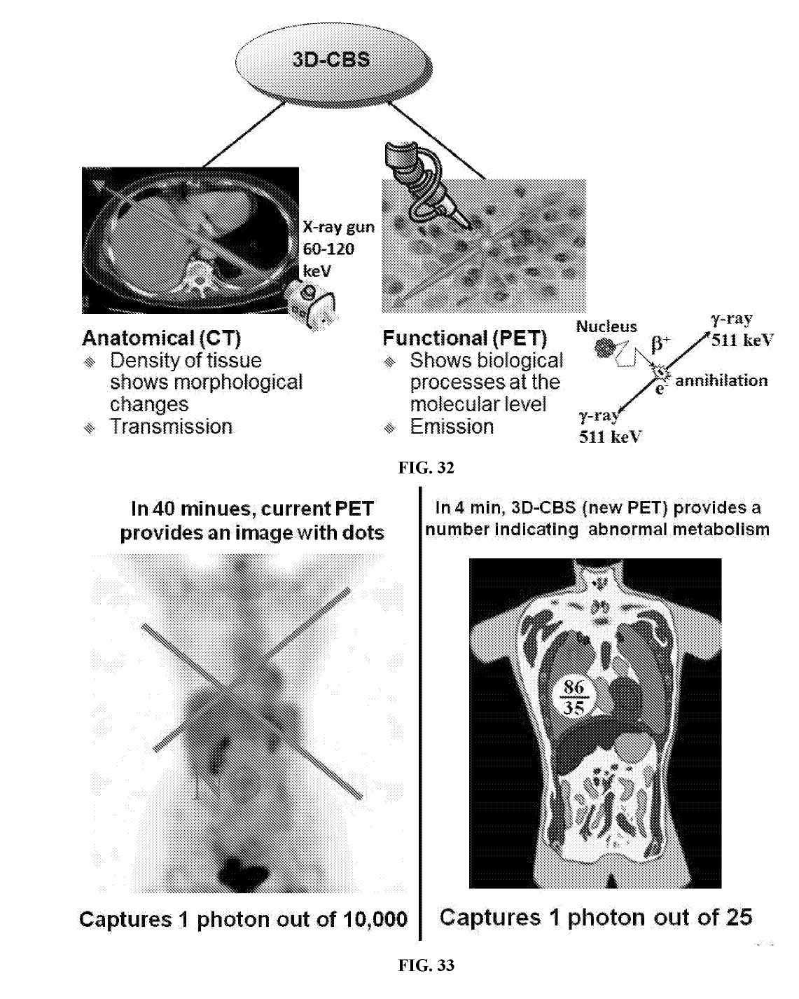

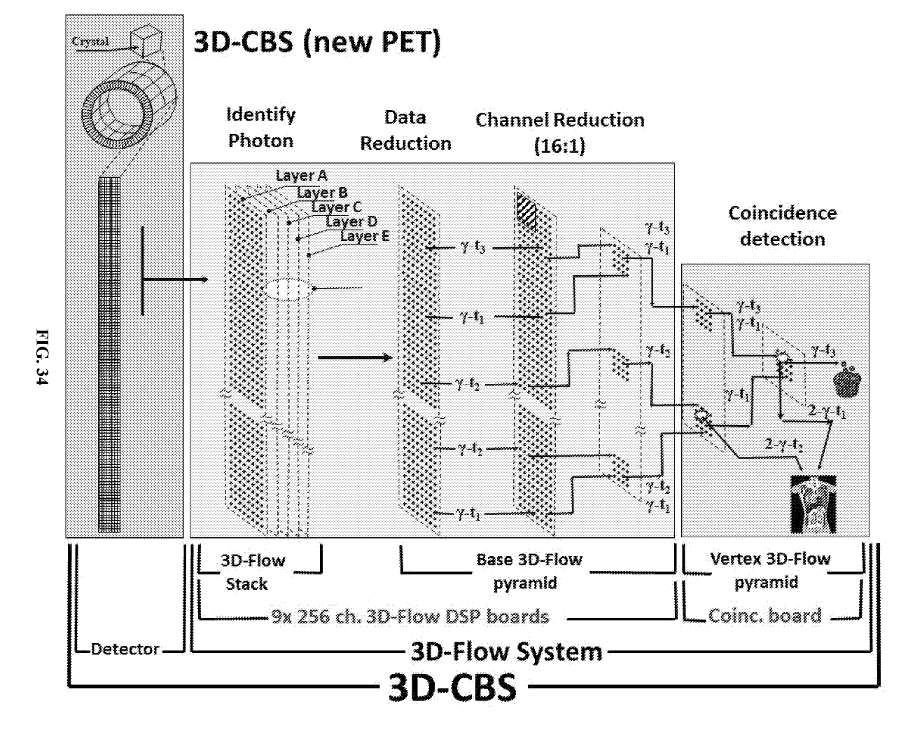

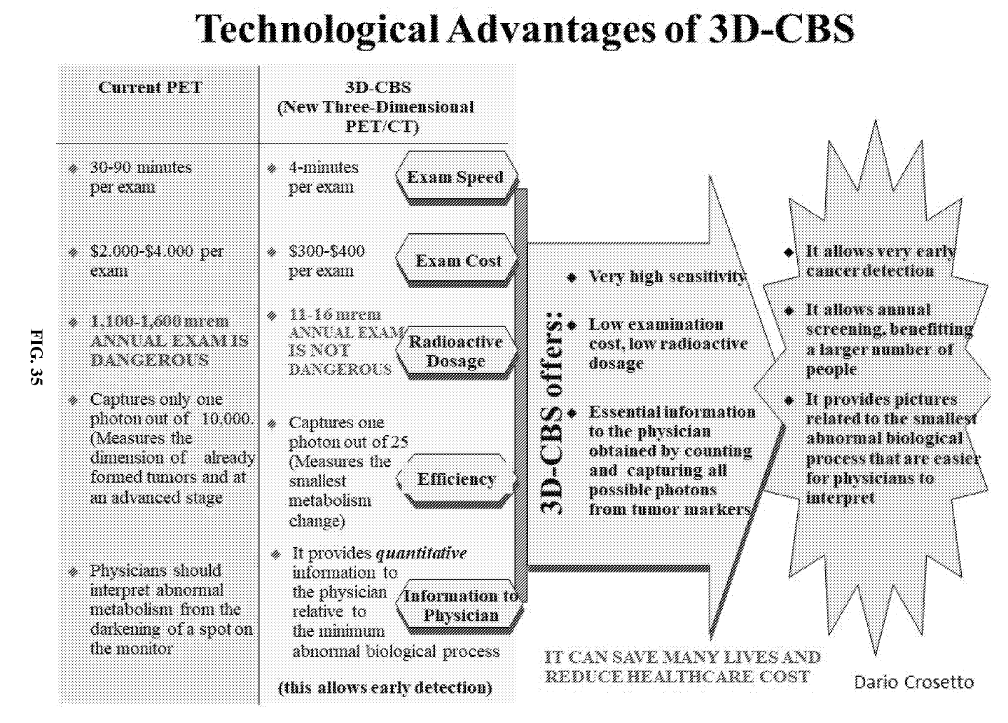

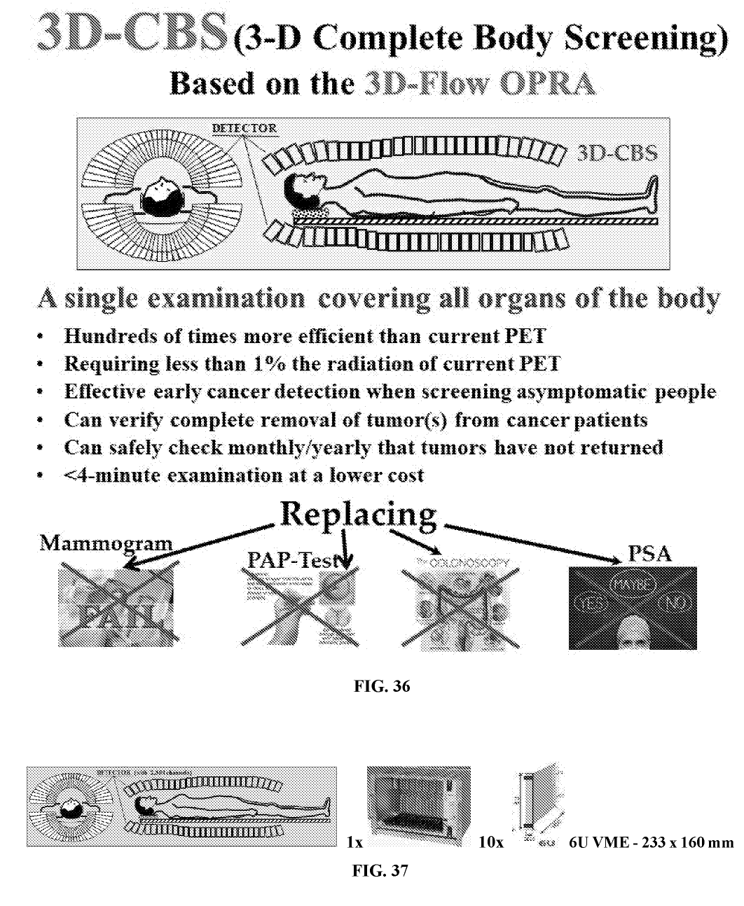

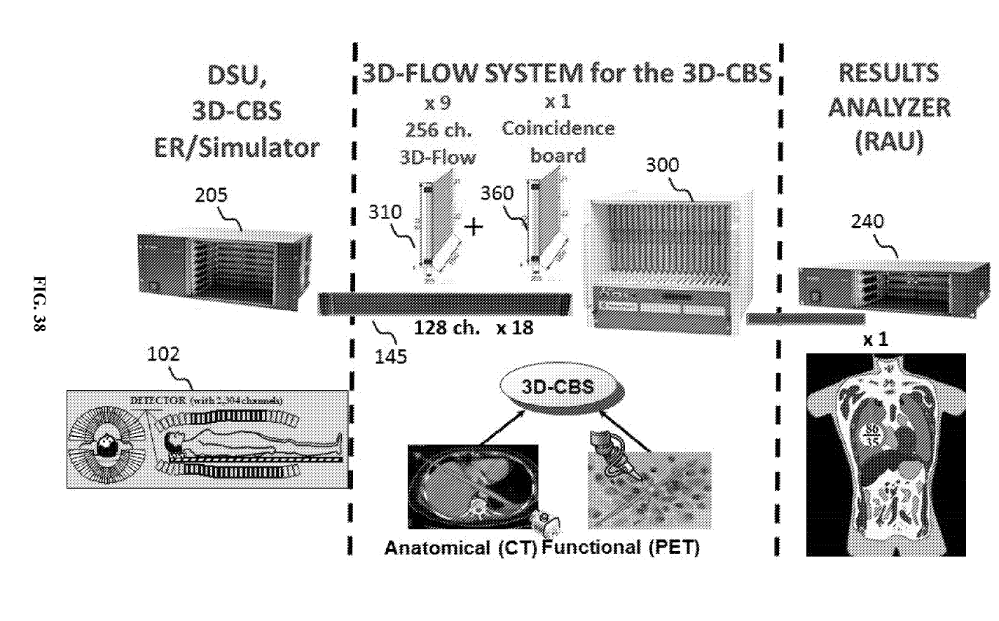

See FIG. 22, FIG. 23, FIG. 24, FIG. 25, FIG. 26, FIG. 27, FIG. 28, FIG. 29.

Following is an example of specifications, a drawing of the logical block diagram, the layout of the components on the board, and a template to prepare the quote for the 3D-Flow VXI form factor electronic board.

The template that I prepared for the different companies that were interested in bidding on this project were the following as reported in the Excel spreadsheet of Table 5

TABLE 5
Excel spreadsheet provided to the companies interested in bidding for
the 3D-Flow VXI form factor electronic board for 84 ASICs per board.

| Item | SUMMARY 9U-VME D-size 366 × 400 mm module | Item | Notes | | |
|---|---|---|---|---|---|
| # ID | DESIGN + NRE + PROTOTYPE | Category | | Volume for | Volume for |
| Item | SMALL/LARGE PRODUCTION | Item | | 20 modules | 20 modules |
| # ID | Description of the material | Category | Qty | (unit cost) | (module cost) |
| 22 | ID = 1, 324 pins, 1 mm pitch, dimensions 19 × | FPGA | 4 | $50.0000 | $200.0000 |
| | 19 mm, or equivalent | | | | |
| 23 | ID = 2, 32-Lead, LQFP, dimensions 10 mm × 10 | Delay-line | 4 | $15.0000 | $60.0000 |
| | mm, power 0.2 W | | | | |
| 24 | ID = 3, 64-Lead, LQFP, dimensions 12 mm × 12 | Clock_Driver | 4 | $25.0000 | $100.0000 |
| | mm, power 1 W | | | | |
| 25 | ID = 4, 20 pin, dimensions 10 mm × 7 mm, | DCDC-REG | 8 | $15.0000 | $120.0000 |
| 26 | ID = 5, 2-pins, dimensions 7 mm × 7 mm, power | Inductance | 8 | $5.0000 | $40.0000 |
| | 0.1 W | | | | |
| 27 | ID = 6, 1681-pins BGA, dimensions 35 mm × 35 | ASIC | 84 | $0.0000 | $0.0000 |
| | mm, power 4.3 W | | | | |
| 28 | ID = 7, 2-pins, resistors 100 ohm for LVDS | Resistors | 13440 | $0.0040 | $53.7600 |
| 29 | ID = 8, 2-pins, capacitors, small | capacitors | 2000 | $0.0800 | $160.0000 |
| 30 | ID = 9, stacking straight connectors carrying 131 | connectors | 4 | $25.0000 | $100.0000 |
| | LVDS front of the board SEAM-30-02.0-S-10- | | | | |
| | 2-A-K-TR or equivalent | | | | |
| 31 | ID = 10, right angle connectors carrying 131 | Connectors | 4 | $25.0000 | $100.0000 |
| | LVDS front of the board SEAM-30-01-S-10-2- | | | | |
| | RA-K-TR, or equivalent | | | | |
| 32 | ID = 11, connectors carrying 700 signals total, | Connectors | 4 | $4.0000 | $16.0000 |
| | rear of the board | | | | |
| 33 | ID = 12, “heat bars” | Heat bars | 10 | $20.0000 | $200.0000 |
| 34 | ID = 13, output connector for 24 LVDS + 8 | Connectors | 1 | $2.0000 | $2.0000 |
| | single-ended signals | | | | |
| | ID = 14, two groups of 8 LED | LED | 2 | $1.0000 | $2.0000 |
| | ID = 15, connector for test points on front panel | Connectors | 1 | $2.0000 | $2.0000 |
| | (32 signals) | | | | |
| 35 | ID = 16, Large capacitors | Capacitors | 400 | $0.5000 | $200.0000 |
| | ID = 17, Miscellaneous | Misc | 1 | $25.0000 | $25.0000 |
| 36 | Purchasing cost 7% of component cost | | | $15.0209 | $96.6532 |
| 37 | ID = 18, PCB cost, dimensions 366 mm × 400 | PCB | 1 | $780.0000 | $780.0000 |
| | mm | | | | |
| 38 | | | | | |

| | | |
|---|---|---|
| 39 | TOTAL BOM Cost per Module | $2,257.4132 |

TABLE 6
Excel spreadsheet provided to the companies interested in bidding for
the 3D-Flow VXI form factor electronic board for 66 ASICs per board

| Item | SUMMARY 9U-VME size 366 mm × 400 mm | Item | Notes | | |
|---|---|---|---|---|---|
| # ID | DESIGN + NRE + PROTOTYPE SMALL | Category | | Volume for | Volume for |
| Item | PRODUCTION | Item | | 20 modules | 20 modules |
| # ID | Description material | Category | Qty | (unit cost) | (module cost) |
| 20 | ID = 1, 324 pins, 1 mm pitch, dimensions 19 × 19 | FPGA | 2 | $50.0000 | $100.0000 |
| | mm, or equivalent | | | | |
| 21 | ID = 2, 32-Lead, LQFP, dimensions 10 mm × 10 | Delay-line | 2 | $15.0000 | $30.0000 |
| | mm, power 0.2 W | | | | |
| 22 | ID = 3, 64-Lead, LQFP, dimensions 12 mm × 12 | Clock_Driver | 2 | $25.0000 | $50.0000 |
| | mm, power 1 W | | | | |
| 23 | ID = 4, 20 pin, dim. 10 mm × 7 mm, or Empirion, or | DCDC-REG | 8 | $15.0000 | $120.0000 |
| | equivalent | | | | |
| 24 | ID = 5, 2-pins, dimensions 7 mm x 7 mm, power 0.1 | Inductance | 8 | $5.0000 | $40.0000 |
| | W | | | | |
| 25 | ID = 6, 1681-pins BGA, dimensions 35 mm × 35 | ASIC | 66 | $0.0000 | $0.0000 |
| | mm, power 4.3 W | | | | |
| 26 | ID = 7, 2-pins, resistors 100 ohm for LVDS | Resistors | 10560 | $0.0040 | $42.2400 |
| 27 | ID = 8, 2-pins, capacitors, small | capacitors | 2000 | $0.0800 | $160.0000 |
| 28 | | | | | |
| 29 | ID = 10, right angle connectors carrying 131 LVDS | Connectors | 4 | $25.0000 | $100.0000 |
| | front of the board SEAM-30-01-S-10-2-RA-K-TR, | | | | |
| | or equivalent | | | | |
| 30 | ID = 11, connectors carrying 700 signals total, rear | Connectors | 4 | $4.0000 | $16.0000 |
| | of the board | | | | |
| 31 | ID = 12, Heat dissipation metal bars | Heat-bars | 8 | $20.0000 | $160.0000 |
| 32 | ID = 13, output connector for 24 LVDS + 8 single- | Connectors | 1 | $2.0000 | $2.0000 |
| | ended signals | | | | |
| | ID = 14, two groups of 8 LED | LED | 1 | $1.0000 | $1.0000 |
| | ID = 15, connector for test points on front panel (32 | Connectors | 1 | $2.0000 | $2.0000 |
| | signals) | | | | |
| | ID = 16, Large capacitors | Capacitors | 400 | $0.5000 | $200.0000 |
| 33 | ID = 17, Miscellaneous | Misc | 1 | $25.0000 | $25.0000 |
| 34 | Purchasing cost 7% of component cost | | | $13.2709 | $73.3768 |
| 35 | ID = 18, Mother-board, dimensions 366 mm × 400 | PCB | 1 | $353.0000 | $353.0000 |
| | mm | | | | |
| 36 | | | | | |

| | | |
|---|---|---|
| 37 | TOTAL BOM Cost per Module | $1,474.62 |

I. The Specifications, Drawings and Requirements I Provided when Requesting Quotes from Different Companies for the LHC TER/DSU Board

I first asked the companies interested in bidding on this project to sign a Non-Disclosure Agreement (NDA), and then provided them the three items listed below. I followed up with several phone calls, emails, and visits to their sites (for companies in Dallas) for a period of two to three months.

I provided:
1. a text description of the specifications.
2. drawings of the logical block diagram showing functionality of the board/chip for which I needed a quote.
3. an Excel spreadsheet with guidelines of the items I suggested in order to have a clear separation between NRE (Non-Recurring Engineering) and a small production of 32 boards, and a larger production that could be purchased in the future from other groups or universities.

As you will notice in the Budget Justification, some companies have filled in the three-page spreadsheet I provided where on the first page there is the summary of the NRE cost which has been separated into the cost of a small production of 17 to 33 electronic boards and a larger production from 100 to 500 boards. Other used their own template to provide their quote.

Following is an example of specifications, a drawing of the logical block diagram, the layout of the components on the board, and a template to prepare the quote for the LHC TER/DSU board.

The template that I prepared for the different companies that were interested in bidding on this project were the following as reported in the Excel spreadsheet of Table 7

TABLE 7
Excel spreadsheet provided to the companies interested in bidding for the LHC TER/DSU board

| | SUMMARY 6U-VME size module 320 MHZ | | |
|---|---|---|---|
| Item | DESIGN + NRE + PROTOTYPE | Item | |
| # ID | SMALL/LARGE PRODUCTION | Category | Notes |
| 1 | NRE Design Module 6U-VME Version #1 | $0.00 | Performed by ?? |
| 2 | NRE PCB layout/fab. Module 6U-VME Version | $0.00 | Performed by ?? |
| | #1 | | |
| 3 | NRE Assembly/test Module 6U-VME Version | $0.00 | Performed by ?? |
| | #1 | | |
| 4 | TOTAL NRE COST VERSION #1 | $0.00 | |
| 5 | Assembly two Module 6U-VME Version #1 | $622.74 | Performed by ?? |
| 6 | Functional Test, Verification and | $0.00 | Performed by ?? |
| | Characterization of two modules Version #1 | | |
| 7 | TOTAL COST OF 2 TESTED MODUL. | $622.74 | |
| | VERSION #1 | | |
| 8 | NRE Design Modifications Module 6U-VME | $0.00 | Performed by ?? |
| | Version #2 | | |
| 9 | NRE PCB Modifications Module 6U-VME | $0.00 | Performed by ?? |
| | Vers. #2 | | |
| 10 | NRE Manufacturing Modifications Module 6U- | $0.00 | Performed by ?? |
| | VME Vers. #2 | | |
| 11 | NRE Testing: Create Functional Pass/Fail Test | $0.00 | Performed by ?? |
| | & Install at Mfg. Facility | | |
| 12 | TOTAL NRE COST VERSION #2 | $0.00 | |
| 13 | Assembly one Module 6U-VME Version #2 | $311.37 | Performed by ?? |
| 14 | Functional Test, Verification and | $0.00 | Performed by ?? |
| | Characterization first module Version #2 | | |
| 15 | TOTAL COST first module Version #2 | $311.37 | |
| 16 | Assembly Module 6U-VME Version #2 | $311.37 | Performed by ?? |
| 17 | Functional Pass/Fail Test each module for 15 | | Performed by ?? |
| | minuntes @ $1.3/min. of the remaining 32 | | |
| | modules Version #2. | | |
| 18 | TOTAL COST of each module of the 32 | $311.37 | |
| | remaining modules of Version #2 | | |
| 19 | Assembly Module 6U-VME, 200 per year | $294.49 | Performed by ?? |
| 20 | Functional Pass/Fail Test each module for 15 | $0.00 | Performed by ?? |
| | minuntes @ $1.3/min. for the production of 200 | | |
| | per year | | |
| 21 | TOTAL COST of each module for the large | $294.49 | |
| | production 200 per year | | |

| | | | | Volume for | Volume for |
|---|---|---|---|---|---|
| Item | | Item | | 35 modules | 35 modules |
| # ID | Description material | Category | Qty | (unit cost) | (module cost) |
| 22 | ID = 1a, 484 pins, 1 mm pitch, dim.s 23 × 23 | FPGA | 8 | $0.0000 | $0.0000 |
| | mm, Power ?? | | | | |
| 23 | ID = 1b, 484, 1 mm pitch, dim. 23 × 23 mm, | FPGA | 1 | $0.0000 | $0.0000 |
| | Power ?? | | | | |
| 24 | ID = 2, 32-Lead, LQFP, dimensions 10 mm × 10 | Delay-line | 2 | $10.0000 | $20.0000 |
| | mm, power 0.2 W | | | | |
| 25 | ID = 3, 64-Lead, LQFP, dimensions 12 mm × 12 | Clock_Driver | 2 | $15.0000 | $30.0000 |
| | mm, power 1 W | | | | |
| 26 | ID = 4, 54 pin, dimensions 10 mm × 7 mm, | DCDC-REG | 4 | $7.0000 | $28.0000 |
| | (Empirion or equivalent) | | | | |
| 27 | ID = 5, 204-pins SODIMM MM80-204B1-1R, | Socket | 4 | $5.5000 | $22.0000 |
| | thin socket | | | | |
| 28 | ID = 6, 204-pins, SODIMM MM80-204B1-E1R, | Socket | 4 | $7.0000 | $28.0000 |
| | tall socket | | | | |
| 29 | ID = 7, 2GB SODIMM DDR3 1600 Mbps | Memory | 8 | $0.0000 | $0.0000 |
| 30 | ID = 9, 2-pins, capacitors, small | capacitors | 100 | $0.0800 | $8.0000 |
| 31 | ID = 10, connectors carrying 131 LVDS front of | Connectors | 4 | $22.0000 | $88.0000 |
| | the board | | | | |
| 32 | ID = 11, VME connectors, backplane | Connectors | 2 | $8.0000 | $16.0000 |
| 33 | ID = 12, user defined backplane connector (P0) | Connectors | 1 | $6.0000 | $6.0000 |
| 34 | ID = 13, 2-pins, resistors | Resistors | 500 | $0.0400 | $20.0000 |
| 35 | ID = 14, Miscellaneous | Misc | 1 | $25.0000 | $25.0000 |
| 36 | Purchasing cost 7% of component cost | | | $7.3934 | $20.3700 |
| 37 | ID = 15, PCB cost, dimensions 233 mm × 160 | PCB | 1 | $0.0000 | $0.0000 |
| | mm | | | | |
| 38 | item . . . ?? (specify ??) | | | | |

| | | |
|---|---|---|
| 39 | TOTAL BOM Cost per Module | $311.3700 |

J. Functional Description of the 320 MHz and 640 MHz ER/DSU Board

### 320 Mhz ER/DSU Board

The primary feature of the 320 MHz ER/DSU board is to transfer data from onboard DDR memory at a maximum rate of 40.96 GB/sec to 512 LVDS DDR channels, for an effective throughput of 640 Mbps for each LVD channel. All 512 bits on the 512 LVDS channels will be presented at the same time, with new being clocked out at a rate of 1.56 ns per bit on the front panel connector. In addition to transmitting data at this speed, the board will be capable of receiving data over the 512 LVDS channels and writing the data to the onboard DDR memory at a rate of 640 Mbps per channel without missing any data.

The 512 LVDS data lines on the front panel are grouped in four connectors, each with 128 LVDS data lines. A separate connector provides control lines such as: External clock, Start transfer and Stop transfer.

The clock determines at which speed the data is transferred. A continuous increment from 1 MHz to 320 MHz (640 Mbps at each channel, presenting at the same time 512-bits on the 512 LVDS channels at a max speed of a new data every 1.56 ns) will not be required, but some frequencies will not be possible if they will be in conflict with the synchronization between read/write with RAS/CAS and other control signals at the DDR3 memory. The “Start Transfer” signal will initiate the transfer to/from the memory of its entire content. At any time the device receiving data can issue a “Stop Transfer”.

While the ER/DSU board is required to transfer data continuously from/to the front panel to/from the memory with no interruption and without missing any data, its interface to the VME bus or to a PCIe host will allow a host computer connected to the same interface to access the memory randomly and/or read and write via a DMA. The amount of data that can be sent or received is limited by the amount of onboard DDR memory. When transmitting, the data in the onboard DDR memory can be transmitted once as a single-shot output, or it can be sent repeatedly in a continuous loop until stopped by the host. In the case that a different data set needs to be transmitted, the host computer will stop transmission and reload the memory with new data. It is envisioned that this reloading would take place at a much slower rate than the 320 MHz/640 MHz rates of the LVDS outputs. Likewise, the receive mode will write incoming data to memory until the memory is full, at which point, the receive logic will stop receiving data and will alert the host that the memory buffer is full. The host computer can then read all of the data from the onboard DDR memory at a reduced data rate before starting to receive new data.

There is no need for arbitration to access the memory from VME (or PCIe) and the front panel connector. The ER/DSU board is either communicating with VME (or PCIe, depending which host is connected to the crate) or it is transferring/receiving data to/from the front panel connector. During these operations VME (or PCIe) cannot access the memory. In order to access the memory, the VME bus (or PCIe) must take control of the memory and disable the communication with the front panel connector.

The ER/DSU board should be accessed from a driver developed for VxWorks and/or from Windows 7 (8 or 10) and should have JTAG. The driver is not a part of this project estimate.

### 640 Mhz ER/DSU Board

The 640 MHz ER/DSU board is similar to the 320 MHz board, with the following differences:
1. The maximum data speed is increased from 320 MHz DDR (640 Mbps/channel) to approximately 640 MHz DDR (1200-1280 Mbps/channel).
2. Each 640 MHz board will only have 2 front panel data connectors instead of 4, each outputting 128 LVDS data channels, for a total output of 256 LVDS channels per 640 MHz board.
3. Different FPGA's and other components will be utilized in order to provide for the faster data transmission and receive rates.
4. All other functionality will match that of the ER/DSU 320 MHz board.

I have also provided specifications, drawings, the layout of the components on the board and the template for the items to quote for the VME board housing 25 chips, each with 64×3D-Flow processors, the VXI board with 66 of these chips and one with 84 chips.

I have also specified which other Integrated circuits are necessary for the ancillary logic, the connectors etc. I verified the feasibility of building each part and made sure that the company could answer questions addressing all aspects relative to the job that needed to be performed.

Notice in the following Excel spreadsheet that I matched the number ID in the PCB board layout with the number ID in the first page of the Excel spreadsheet in the Build of Material (BOM).

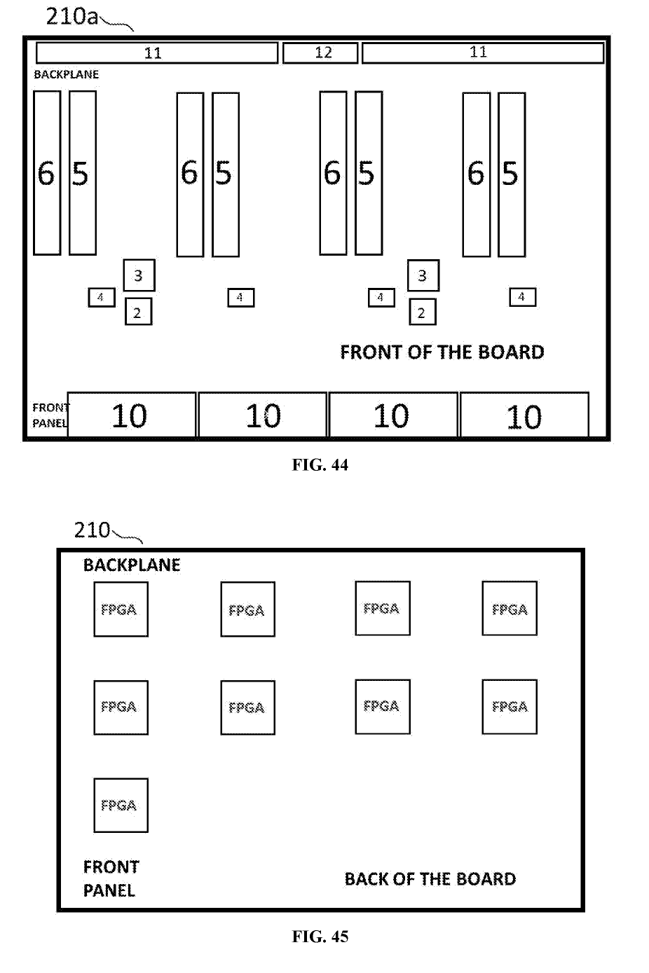

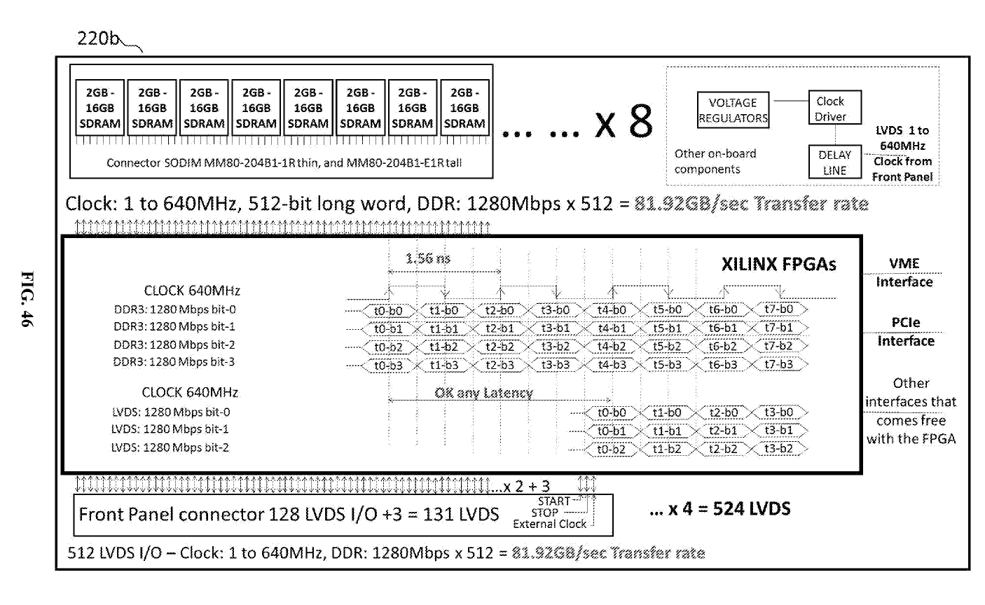

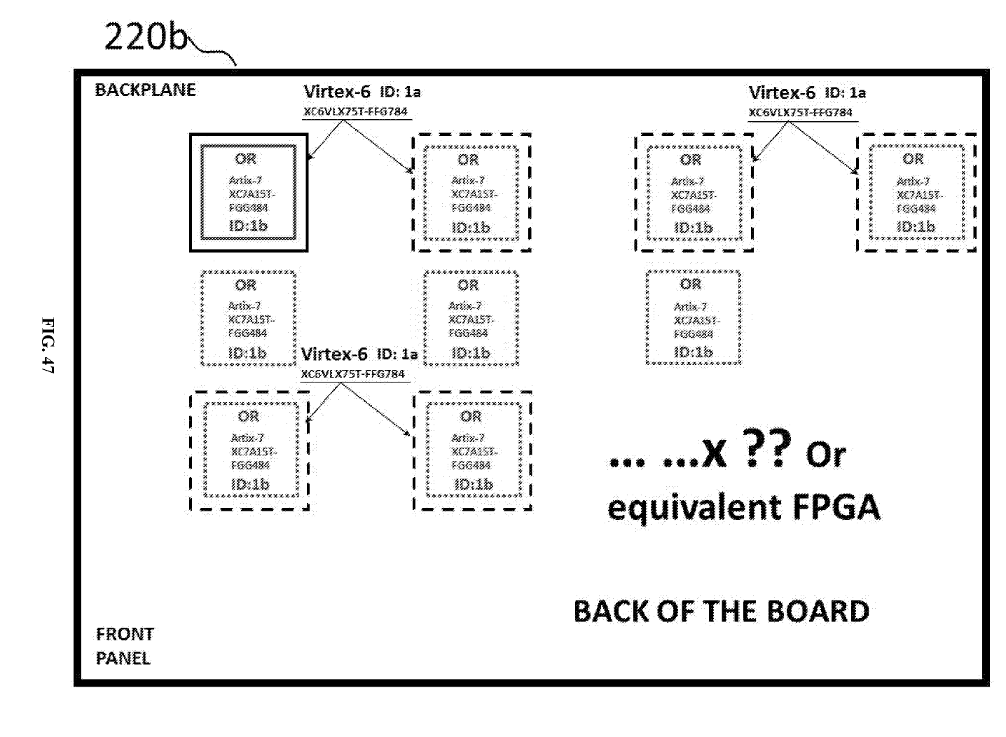

See FIG. 43, FIG. 44, FIG. 45, FIG. 46, FIG. 47.

K. Step-by-Step Development of the Project Showing Feasibility and Functionality

My professional experience in the field lead me to approach the problem by looking at the objectives of creating a system with the capability to identify objects (through pattern recognition) satisfying experimenter's desired characteristics (modifiable through a programmable complex real-time algorithm) from data arriving at a speed up to 1.3 TB/sec, propose a solution that offers deliverables of measurable results on a test bench of a lab and ease integration into the target system.

I therefore observed the entire HEP application system for 8,192 channels at 1.3 TB/sec transfer rate in FIG. 4 and the medical imaging application for 2,304 channels at 368 GB/sec transfer rate in FIG. 6 and identified the key parameter on which I needed to focus the data rate for a single channel at source and destination which is 0.16 GB/sec, which is equivalent to 1280 Mbps (160 MB/sec×8-bit=1280 Mbps).

Whether CERN decides to bundle several of these 8,192 signals together and transport 10 of these on a cable or optical fiber at 10 times the speed, or 100 at 100 times the speed will be determined by distance, environment and cost/performance of 10 GbE, vs. 100 GbE, vs. SATA, etc.; however, data integrity at destination should reflect the same content and integrity of the signals at the source generated at 1280 Mbps per channel, equivalent to data commuting every 781 picoseconds (ps).

This speed of data arriving at each channel every 781 ps satisfies the requirements for extracting all valuable information from radiation with a system like the 3D-Flow having the capability to execute Object Pattern Real-Time Recognition Algorithms on multiple data arriving at this speed. It satisfies the need for future upgrades of the LHC either running at frequency higher than the actual 40 MHz or at a higher luminosity. According to articles published by CERN, the LHC could not run at 80 MHz due to beam heating, however, it is planned to run at a higher luminosity.

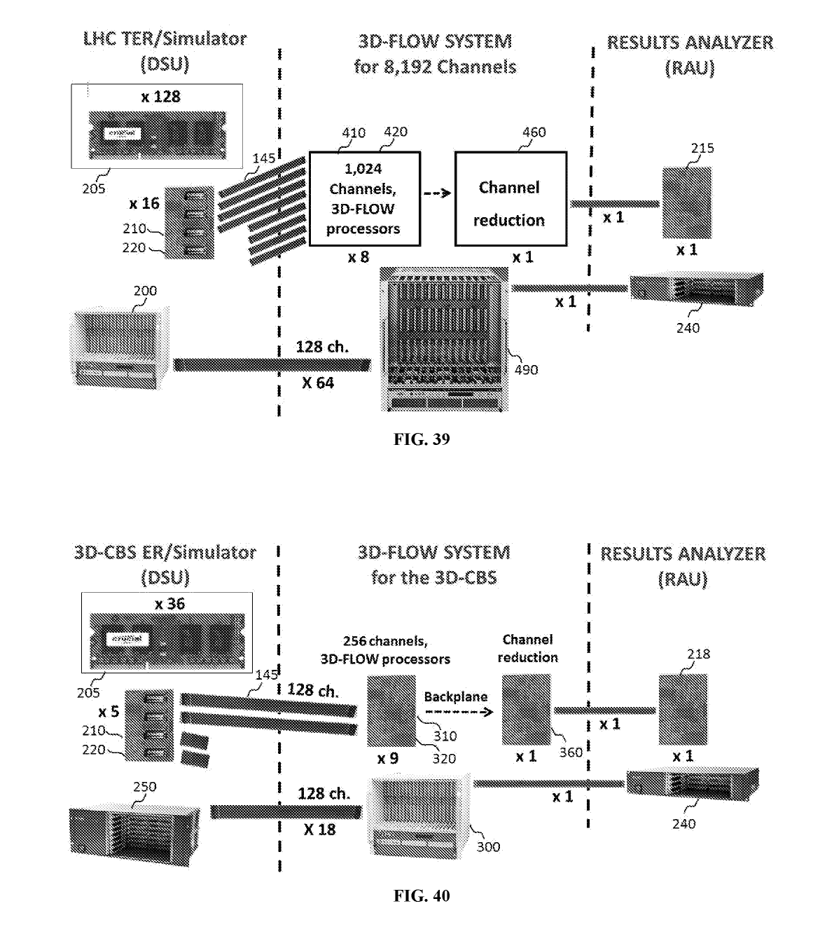

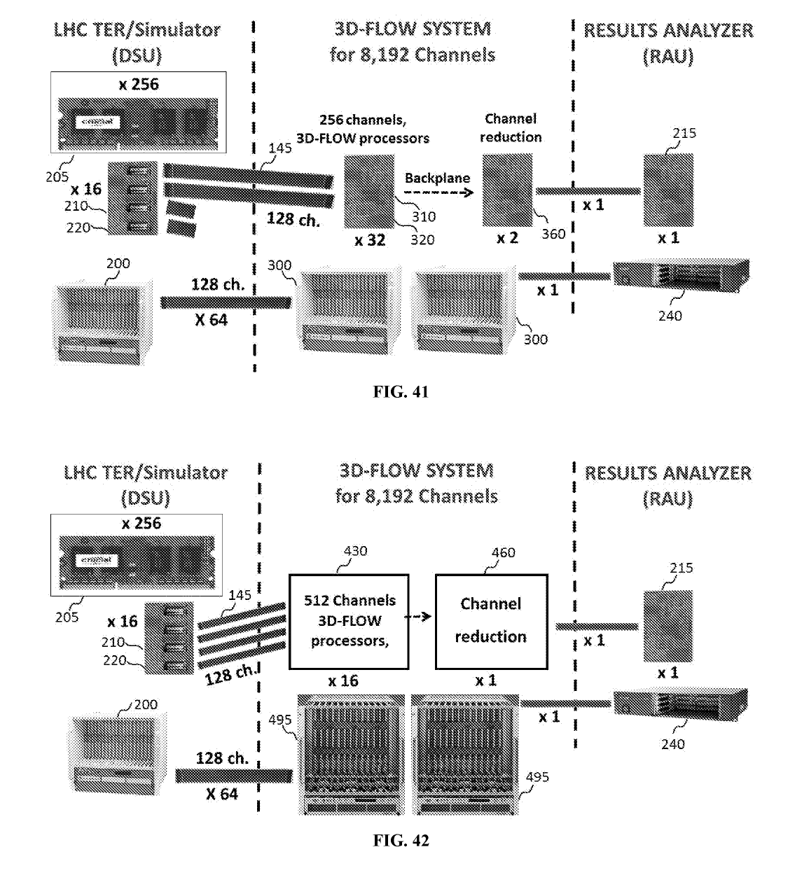

See FIG. 39 for an overview of the test bench system implemented using the VXI form factor and FIG. 41 implemented using the VME form factor that will allow experimenters' to test their Level-1 Trigger system (3D-Flow system or any other Level-1 Trigger approach).

Creating a 3D-Flow Trigger system with 781 ps data rate capability at each channel will satisfy either acquiring 16-bit data from the LHC at 80 Mhz if the beam heat problem is solved (16×781 ps=12.5 ns) or 32-bit data from LHC at 40 MHz generating more data with a higher luminosity that is planned for future upgrades at CERN as stated in their documents (32×781 ps=25 ns LHC bunch-crossings).

The planned workflow of this proposal that will optimize the use of resources and taxpayer money to build a system that can provide measurable results on a test bench of a laboratory and easily integrate into applications such as LHC, Medical Imaging, etc., is to advance from purchasing instrumentation available on the market to generate and record/display signals similar to those generated by radiation at LHC, medical imaging, video cameras, etc. for two signals and create instrumentations to generate and record/display 8,192 signals.

The instrument from a vendor catalogue at $55,000 generating 2 signals similar to those generated from radiation at LHC, medical imaging, etc., will allow 512 and 256 channel VME boards generating similar signals to be built.

At this point the 512 and 256 channel VME boards costing about $2,250 each to duplicate, become the instruments to test equivalent VME boards recording/displaying 512 and 256 channels at that speed.

Sixteen 512 channel VME boards for a total cost of approximately $40,000 for 8,192 channels in a VME crate become the DSU (Detector Simulator Unit) instrument providing the signals similar to (with a little additional performance in providing 16-bit information for each data instead of the current 10-bits provided by LHC experiments) to those provided by the LHC experiments until 2018.

This DSU 8,192 channel instrument replaces the Tektronix waveform generator and becomes the signal generator to test an equivalent 8,192 channel TER unit (Trigger Event Recorder).

Once we have tested the 8,192 channel TER unit, we can take this to CERN to record raw trigger data at LHC and send the data to Scientific Associates of the universities collaborating to the experiment who can use real data on a DSU unit reproducing all the functionality as data generated at the LHC to test the performance of the 3D-Flow system (or any other trigger system) in finding the rare Higgs boson-like particle, or any particle that theoretical physicists can provide characteristics for, that will then be translated into real-time algorithms to be downloaded into the 3D-Flow system (or any other trigger system). The Scientific Associates can even edit the raw data and create very difficult conditions such as adding pileup events, adding noise generated by spurious particles, etc., to verify the efficacy of the 3D-Flow system (or any other trigger system) to filter the particles matching the characteristics set by the theoretical physicists or experimenters for the new particle.

What we will have accomplished at this stage is to have created a 512 channel board and an 8,192 channel DSU and TER from the basic 2 to 4 channel instrumentation purchased on the market for approximately the same cost, when duplicated, as the 2 channel instrument available on the market. They can be used to test each other (first one crate is set to work as a DSU and the other as TER, next the functions of both are swapped, so the functionalities on both are verified).

At the same time we have created two new instruments to be used at CERN to record raw data trigger events and as an 8,192 channel parallel data generator, generating signals similar to those generated by LHC to be used on test benches at laboratories of Scientific Associates located remotely from CERN to test the 3D-Flow system (or any trigger system).

The same occurs when building the 256 channel VME board at 1280 Mbps per channel. Building two such boards with the help of the Tektronix waveform generator and oscilloscope will allow to both VME boards to be verified when set to work as DSU or as TER or vice-versa. Similarly, building two crates, each with 16×256 boards will allow the system to be tested for 4,096 channels, one case working as a DSU and the other as TER and vice-versa. The test will be considered complete only with 4,096 channels because what is important to test is the signal integrity, crosstalk free, power dissipation and the capability to work at full speed for several hours. Additional crates coupled one-to-one to build systems of 8,192 channels, 12,288 channels or 16,384 channels will present additional challenges in power dissipation, signal integrity, etc. because all these characteristics and challenges will be confined within the crate.

A more detailed flow of the work for this proposal follows:
1. Build a DSU test bench for 2 channels, at 1280 Mbps each. Generate 2 signals (cost of instrument from vendor catalogue is $55,000. This arbitrary waveform generator is all I strictly need for this project, although the catalogue does offer instruments with higher performance costing up to $120,000).
   DONE I have identified a commercially available instrument made by Tektronix, Model AWG5012C, 1.2 Gsamples per second, 14-bit that can generate signals on 2 channels similar to those generated by the radiation at LHC, a PET device, a CT scan, multi-lens movie cameras, etc.
   The instrument costs approximately $55,000.
2. Build a TER test bench for 2 channels, at 1280 Mbps each. Record and display 4 signals (cost of instrument from vendor catalogue is $65,000. This oscilloscope at 4 GHz is all I strictly need for this project, although the catalogue does offers oscilloscopes at 33 GHz with higher performance, larger memory, etc., costing >$450,000).
   DONE I have identified an oscilloscope made by Tektronix, model MSO70404C, 4 GHz with 4 analogue channels and 16 digital channels that could record and display all parameters relative to signals with an “eye” of 781 picoseconds.
   The instrument and probes cost approximately $65.000.
3. Design a TER/DSU test bench board for 512 channels, at 640 Mbps each for LHC 2017-18, Medical Imaging, and other applications. Generate and record signals to/from a sequence of data stored into memory (cost for each duplicate instrument built with this proposal is $2,250). Design a TER/DSU VME board for 512 channels @ 640 Mbps per channel. Contact reputable companies to provide a quote for the cost of an NRE to manufacture electronics boards offering a turnkey solution.
   DONE.
   NEEDED funds to pay the company for the implementation of the NRE at their average rate of $ 110/hour.
4. Build a TER test bench for 102 channels, at up to 1280 Mbps each. Record and display 102 signals (cost of Instrument from vendor catalogue is $160,000. This Logic State Analyzer at 50 GHz is all I strictly need for this project, although the catalogue offers Logic State Analyzers with higher performance, memory, etc., costing >$200,000).
   DONE I have identified a Logic State Analyzer and relative probes made by Tektronix, model TLA7012, 50 GHz, 102 channels, that can test for signal integrity, crosstalk between signals, etc. on busses with several data lines and signals in parallel at a DDR3 memory interface; it can also record and display characteristics of all parameters relative to 102 signals in relation to one another with probes for a SODIMM slot interposer.
   NEEDED funds to pay for the instrument and probes costing approximately $160,000.
5. Build a DSU test bench board for 512 channels, at 640 Mbps each for LHC 2017-18, Medical Imaging, and other applications. Generate signals using the arbitrary waveform generator AWG5012C and using data stored in memory and send them to the front panel connectors @640 Mbps of the DSU unit (cost for each duplicate instrument built with this proposal is $2,250). Using the arbitrary waveform generator AWG5012C, the oscilloscope MSO070404C, and the Logic State Analyzer TLA7012, work with the company that has manufactured the VME 512 channel board @ 640 Mbps to test it as a DSU unit. Hire personnel selected in a competitive hiring process at an average cost of $38/hour, which includes undergraduate students, graduate students, and postdoctoral researchers, to be trained in this field to work with the company to test the 512 channel board @640 Mbps as a DSU unit. Students of physics, electronic engineering, and computer science will interact with the company testing the VME board, possibly spending some time at their facility. The company will benefit from the students performing some routine electronic tests and from the development of software which tests the board, and the students benefit by learning from experienced engineers in the field.
6. Build a TER test bench board for 512 channels, at 640 Mbps each for LHC 2017-18, Medical Imaging, and other applications. Record signals from a sequence of data arriving from 512 channels at the front panel connectors at 640 Mbps into memory (cost for each duplicate instrument built with this proposal is $2,250). Using the DSU board tested in the previous phase of this proposal as the instrumentation in place of the arbitrary waveform generator AWG5012C, work with the company that has manufactured the VME 512 channel board @ 640 Mbps to test this second board as a TER unit. Hire personnel selected in a competitive hiring process at an average cost of $38/hour, which includes undergraduate students, graduate students, and postdoctoral researchers, to be trained in this field to work with the company to test the 512 channel board @640 Mbps as a TER unit. Students of physics, electronic engineering, and computer science will interact with the company testing the VME board, possibly spending some time at their facility. The company will benefit from the students performing some routine electronic tests and from the development of software to test the boards, and the students benefit by learning from experienced engineers in the field
7. Build a DSU test bench crate for 8,192 channels, at 640 Mbps each for LHC 2017-18, Medical Imaging, and other applications. Generate a sequence of signals from the data stored in memory and send them in parallel every 1.56 nanosecond (ns) to the 8,192 channels at the front panel connectors of the 16×DSU boards (cost for each duplicate instrument built with this proposal is $40,000). Design a DSU VME system for 8,192 channels @ 640 Mbps per channel. Hire personnel selected in a competitive hiring process at an average cost of $38/hour, which includes undergraduate students, graduate students, and postdoctoral researchers, to be trained in this field to integrate 16×DSU VME boards in a crate, purchase a VME CPU, a VME crate and other instrumentation, test the overall hardware, develop software, etc.
   Each university and research center involved in the 2017 Level-1 trigger upgrade of any LHC experiment should have on their laboratory's test bench this DSU instrument costing only $40,000. This would be like having the trigger data generated by the LHC experiment (collider plus detector) costing billions of dollars at their fingertips but at a fraction of the cost. They could test the efficacy of their trigger electronics in executing Object Pattern Real-Time Recognition Algorithms (OPRA) with zero dead-time on data arriving from the DSU unit at 640 Mbps per channel from 8,192 channels as if they were generated by the actual LHC experiment.
   Having a DSU instrument on their laboratory's test bench to test their trigger electronics would avoid wasting millions of dollars building Level-1 Trigger electronics without sufficient capability to execute complex OPRA able to extract all valuable information from new particles, filtering the background noise caused by a higher luminosity at LHC until 2018 and able to resolve pileup events.
   This would also avoid the additional wastage of billions of dollars and millions of hours of work by the over 10,000 people who built other electronics and analyzed useless data because the Level-1 Trigger did not have the capability to select the good events by executing complex OPRA at zero dead-time. This would further avoid discovering that after more than 20 years of work by over 10,000 people and spending billions of dollars, the CMS and Atlas detectors at LHC did not work as expected as it could find only 40 Higgs boson-like particles casually recorded from among the 1,000 trillion events generated by the LHC Collider when 100,000 were estimated to be Higgs boson-like. Even those who built the trigger (CMS and Atlas) admit at conferences and in official documents describing the upgrade of CMS and Atlas that the electronics of the Level-1 trigger needs to be trashed because it does not have object pattern recognition capability.
   This DSU is a valuable instrument for experimenters to create a low-cost controlled environment in their lab capable of not only using raw trigger data recorded at the CERN experiments but also having the possibility to edit the data manually, adding the most difficult patterns they may think could occur to test the efficacy of the 3D-Flow Trigger system (or the Level-1 Trigger system they have developed).
   To avoid re-inventing the wheel . . . , before designing the DSU and TER boards, crates and system, I checked if there was a similar board commercially available. The closest that I could find was the VPX board of quote #36c (see quote #36c on page 153 of the Budget Justification), consisting of 32 channels @ 250 Mbps per channel and costing $12,595. To build 8,192 channels @ 250 Mbps per channel, requires 256 of these VPX 57610 boards and 22 VPX crates costing approximately $8,000 each to house them. The total cost using the commercially available boards would cost over S3.4 million.
   My TER/DSU boards and system of 512 channels per board @ 640 Mbps per channel for any system size has a patent pending. I am willing to offer 80% of my patent licenses to philanthropists and/or investors who will commit to using a percentage of the income generated to pay for free cancer screening examinations for low income people.
8. Build a TER test bench crate for 8,192 channels, at 640 Mbps each for LHC 2017-18, Medical Imaging, and other applications. Record signals from a sequence of data each arriving @ 640 Mbps in parallel from 8,192 channels at the front panel connectors of the 16×TER VME boards (cost for each duplicate instrument built with this proposal is $40,000). Using the DSU board tested in the previous phase of this proposal as the instrumentation, design, build and test a second crate with 16×TER VME system for 8,192 channels @ 640 Mbps per channel. Hire personnel selected in a competitive hiring process at an average cost of $38/hour, which includes undergraduate students, graduate students, and postdoctoral researchers, to be trained in this field to integrate 16×TER VME boards in a crate, purchase a VME CPU a VME crate and other instrumentation, test the overall hardware, develop software, etc.
9. Swap DSU and TER functionality on a test bench crate for 8.192 channels, at 640 Mbps each for LHC 2017-18, Medical Imaging, and other applications. Swap the functionality of the two DSU and TER units tested earlier. Make sure that each crate with 8,192 channels can work either as a DSU unit or as a TER unit (cost for each duplicate instrument built with this proposal is $40,000: TER and DSU). Change the functionality of the 8,192 channels, 16×DSU boards in the crate tested earlier to 8,192 channels, 16×TER boards by changing the FPGA code, e.g. inserting a 100 ohm termination resistor at each LVDS receiving line. Change the functionality of the 8,192 channels, 16×TER boards in the crate tested earlier to 8,192 channels, 16×DSU boards by changing the FPGA code, e.g. removing a 100 ohm termination resistor at each LVDS sending line. Hire personnel selected in a competitive hiring process at an average cost of $38/hour, which includes undergraduate students, graduate students, and postdoctoral researchers, to be trained in this field to fully test both functionalities: TER and DSU on the entire crate of 8,192 channels.
   These two new instruments will provide a valuable tool to over 12,000 people working on the LHC experiments at CERN to test the efficacy of their Level-1 Trigger system by placing one TER unit at CERN recording raw Trigger data and sending the recorded data to the Scientific Associates at several universities equipped with a DSU unit.
   These Scientific Associates will have the great advantage of being able to recreate in their lab with only $40,000 the real-time environment of the particle event generator from the detectors at CERN costing billions of dollars. They could use raw trigger data recorded from real experiments at CERN, edit manually the data to include even more difficult situations like high background noise and pileup events, test the functionality and efficiency of their hardware by implementing the Level-1 trigger, improve their electronics and algorithms to assure they have the capability to identify new particles having the characteristics sought by experimenters or provide conclusive test results disproving a theory.
   These two instruments will largely satisfy the needs of the most stringent requirements of experiments running at LHC until 2018. Thereafter, they will still be useful to develop and test the efficiency of any trigger system at a very low cost and help develop the new algorithm and Level-1 Trigger systems for experiments with higher luminosity at LHC after 2019. These should be tested for full performance with the two new DSU and TER units capable of sustaining the double speed of 1280 Mbps per channel described in the next items.
   Building these two instruments is very important because it would save billions of dollars and the time of thousands of engineers and scientists who would otherwise continue for years to build detectors and electronics churning out non-valuable information recorded by inefficient Level-1 Triggers and then spend years analyzing this non-valuable data that could not be tested on a bench of a laboratory.
10. Design a TER/DSU test bench for 256 channels, at 1280 Mbps each for LHC 2020, medical imaging and other applications. Generate and record signals from/to a sequence of data stored into memory (cost for each duplicate instrument built with this proposal is %2,500). Design a TER/DSU VME board for 256 channels @ 1280 Mbps per channel. Contact reputable companies that would provide a quote for an NRE cost to manufacture electronics boards offering a turnkey solution.
   DONE (quote available from several companies).
   NEEDED funds to pay the company implementing the NRE at their average rate of $110/hour.
11. Follow same steps from 5 to 9 to develop 16×DSU units, 256 channels boards at 1280 Mbps, 16×TER units, 256 channels at 1280 Mbps, one DSU crate with 4,096 channels at 1280 Mbps, one TER crate with 4,096 channels. Test full functionality of both crates at maximum speed for several hours.
12. Build a DSU test bench crate for 4,096 channels, at 1280 Mbps each for LHC 2020, medical imaging and other applications. Generate a sequence of signals from the data stored in memory and send them in parallel every 781 picosecond (ps) to the 4,096 channels at the front panel connectors of the 16×DSU boards (cost to duplicate each instrument of 8,192 channels built with this proposal is $130,000). Design a DSU/VME system for 4,096 channels @ 1280 Mbps per channel. Hire personnel selected in a competitive hiring process at an average cost of $38/hour, which includes undergraduate students, graduate students, and postdoctoral researchers, to be trained in this field to integrate 16×DSU VME boards in a crate, purchase a VME CPU, a VME crate and other instrumentation, test the overall hardware, develop software, etc. (see Budget Section for details, quotes 18a on page 109 of BJ and 18b on page 141 of BJ).
   Each university and research center involved in the Level-1 trigger upgrade of any LHC experiment for data taking after the year 2020 with much greater luminosity, with additional sub-detectors providing more information to the Level-1 Triggers and requiring the transfer of more bits per channels at 40 MHz LHC bunch-crossing, should have on their laboratory's test bench this DSU instrument costing only $130,000. This would be like having the trigger data generated by the LHC experiment (collider plus detector) costing billions of dollars at their fingertips but at a fraction of the cost. They could test the efficacy of their trigger electronics in executing Object Pattern Real-Time Recognition Algorithms (OPRA) with zero dead-time on data arriving from the DSU unit at 1280 Mbps per channel from 8,192 channels as if they were generated by the actual LHC experiment.
   Having a DSU instrument on their laboratory's test bench to test their trigger electronics would avoid wasting millions of dollars building Level-1 trigger electronics without sufficient capability to execute complex OPRA able to extract all valuable information from new particles, filter the background noise cause by the much greater luminosity at LHC beginning in 2020, able to resolve pileup events and to handle more data from additional sub-detectors.
   This would also avoid the additional wastage of billions of dollars and millions of hours of work by the over 12,000 people who built other electronics and analyzed useless data because the Level-1 trigger did not have the capability to select the good events by executing complex OPRA at zero dead-time. This would further avoid discovering that after more than 20 years of work by over 10,000 people and spending billions of dollars, the CMS and Atlas detectors at LHC did not work as expected as they could find only 40 Higgs boson-like particles casually recorded from among the 1,000 trillion events generated by the LHC Collider when 100,000 were estimated to be Higgs boson-like. Even those who built the trigger admit it needs to be trashed because it does not have object pattern recognition capability.
   This DSU is a valuable instrument for experimenters to create a low-cost controlled environment in their lab capable of not only using raw trigger data recorded at the CERN experiment, but also have the possibility to edit data manually, adding the most difficult pattern they may thing could occur to test the efficacy of the 3D-Flow trigger system (or the Level-1 Trigger system they have developed).
   To avoid re-inventing the wheel . . . , before designing the DSU and TER boards, crates and system, I checked if there was a similar board commercially available. The closest that I could find was the VPX board of quote #36c (see quote #36c on page 153 of the Budget Justification), consisting of 32 channels @ 250 Mbps per channel costing $12,595. To build 8,192 channels @250 Mbps per channel requires 256 of these VPX 57610 boards and 22 VPX crates costing approximately $8,000 each to house them. The total cost using the commercially available boards would cost over $3.4 million
   My TER/DSU boards and system at 256 channels per board @ 1280 Mbps per channel for any system size has a patent pending. I am willing to offer 80% of my patents licenses to philanthropists and/or investors who will commit to using a percentage of the income generated to pay for free cancer screening examinations for low income people.
   NEEDED: Funds to purchase additional items listed in the budget section and to pay our team of 12 people an average rate of $38/hour.
13. Swap DSU and TER functionality on a test bench crate for 4,096 channels, at 1280 Mbps each for LHC 2020, medical imaging and other applications. Swap the functionality of the two DSU and TER units tested earlier. Make sure that each crate with 4,096 channels can work either as a DSU unit or as a TER unit (cost for each duplicate of an 8.192 channel instrument built with this proposal is $130,000: TER and DSU). Change the functionality of the 4,096 channels, 16×DSU boards in the crate tested earlier to 4,096 channels, 16×TER boards by changing the FPGA code, e.g. insert 100 ohm termination resistor at each LVDS receiving line. Change the functionality of the 4,096 channels, 16×TER boards in the crate tested before to 4,096 channels, 16×DSU boards by changing the FPGA code, e.g. remove 100 ohm termination resistor at each LVDS sending line. Hire personnel selected in a competitive hiring process at an average cost of $38/hour, which includes undergraduate students, graduate students, and postdoctoral researchers, to be trained in this field to fully test both functionalities: TER and DSU on the entire crate for 4,096 channels.
   In order to prove full feasibility and functionality of the Triggers' 8,192 channels for CERN's CMS and ATLAS experiments at 1280 Mbps per channel, twice the speed of previous versions of DSU and TER at 640 Mbps, it would be sufficient and would save money to build and test only two crates containing 4,096 channels because all tests are exhausted within the crate (power consumption, crosstalk, speed, signal integrity, etc.). Both crates when set to work as DSU units would provide 8,192 channels, sufficient to test the functionality of a complete 3D-Flow Level-1 trigger system (or any other trigger system) for the largest LHC experiments. These TER and DSU instruments are defined within a crate, but in the previous case of the lower speed there were 8,192 channels per crate and therefore it was necessary to build 16,384 channels to complete all tests.
   It is very important to build these two instruments because it would save billions of dollars and the time of thousands of engineers and scientists who would otherwise continue for years to build detectors and electronics churning out non-valuable information recorded by inefficient Level-1 triggers and then spend years analyzing this non-valuable data that could not be tested on a bench of a laboratory.
   These two new DSU and TER instruments at double speed would provide a valuable tool to the over 12,000 people preparing the construction of a more powerful Level-1 Trigger system for the experiments at LHC with a higher luminosity planned to be operational in 2020.
   The Associates, members of the large LHC experiments, will have the great advantage of being able to recreate in their lab with only $100,000 the real-time environment of particle events that will be generated from the detectors at CERN costing billions of dollars even before its construction. They could use raw trigger data from real experiments previously recorded at CERN, edit manually the data to include even more difficult situations like high background noise and pileup events, test the functionality and efficiency of their hardware by implementing the Level-1 trigger, improve their electronics and algorithms to assure they have the capability to identify new particles having the characteristics sought by experimenters or provide conclusive test results disproving a theory.
14. Demonstrate the 3D-Flow invention is technology-independent, easily migrating to future technologies having the advantage of lowering the price and increasing performance as technology improves.
   DONE. During the implementation of the DOE $906,000 grant from 1995 to 1998, I proved the 3D-Flow design to be technology-independent by compiling the 3D-Flow processor/chip in three different FPGA (Field Programmable Gate Array) technologies (ORCA, from Lucent, Xilinx and Altera) and to a standard cell 350 nanometer (nm) CMOS technology using Synopsys tools. I also paid them as a consultant to provide the tape-out file to be sent to the silicon foundry that would manufacture the chip (Synopsys is recognized as having one of the best software tools and being one of the best ASIC Design companies in the world). Upon request, I can provide the gate count, speed performance, and power dissipation for the four designs.
   Unfortunately, the funds to pay the NRE to the silicon foundry to make the 3D-Flow chip with 350 nm technology was never provided, and although Synopsys tools, because of the success they have had with many designs in the past, guarantees that the silicon chip would have the same characteristics and performance as the design they provided in the tape-out file to the foundry, we could never test the chip as it was never funded. Later, in 2003, I used my own savings and money I received from friends, to implement four 3D-Flow processors in an Altera FPGA that was tested in Altera prototypes boards and later included in a modular industrialized IBM PC board with 68×3D-Flow processors. See in the following steps.
   NEEDED to advance science and for saving lives: a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.
15. Demonstrate feasibility to implement a 3D-Flow chip in 350 nanometer technology with 4×3D-Flow processors per chip
   DONE. During the implementation of the DOE $906,000 grant from 1995 to 1998, I proved feasibility to implement four 3D-Flow processors in a chip with 350 nanometer technology. Synopsys, the consultant who generated the tape out files to be sent to the foundry for the fabrication of the chip with 3D-Flow processors successfully simulated each processor at 61 MHz (Synopsys is recognized as having one of the best software tools and being one of the best ASIC Design companies in the world). This allowed to execute more than one cycle of up to 26 operations such as add, subtract, compare one value with 24 values, etc. and the bypass switch of the 3D-Flow architecture, together with the stacking assembly of several daughter boards of the 3D-Flow system already built in 1994 at the Superconducting Super Collider allowed to increase the number of cycles to implement any experimenters' complex real-time algorithm at the Level-1 Trigger.
   Synopsys provided a report of silicon characteristics of each 3D-Flow processor synthesized in 350 nanometer technology as follows: Logic 2,565,989 units*0.0378=96,940; Memory 2,247,513 equivalent to 90K gates.
   Unfortunately, the funds to pay the NRE to the silicon foundry to make the 3D-Flow chip with 350 nm technology was never provided, and although Synopsys tools, because of the success they have had with many designs in the past, guarantees that the silicon chip would have the same characteristics and performance as the design they provided in the tape-out file to the foundry, we could never test the chip as it was never funded.
16. Develop the software tools to simulate thousands of processors in C++, four processors in a chip at the gate level in VHDL, and the real-time design software tool to allow users to create a project of different size and complexity, simulate it, verify the performance and feasibility from user system algorithm to gate-level circuits, interface the 3D-Flow software tools to third-party Electronic Design Automation (EDA) tools, allow implementation of user's conceptual idea into the fastest programmable system at the gate circuit level, etc.
   DONE. THE 3D-Flow Design Real-Time software tools were developed with a DOE grant from 1995 to 1998
   NEEDED to advance science and for saving lives: funds to pay our team of 12 people to do the porting of all this software from NT operating system and the software environment when it was developed in 1996 to Windows 10, Linux. OS-2. MsWorks, to write user manuals of the software to facilitate users.
17. Demonstrate the 3D-Flow system with the capability to execute Level-1 Trigger algorithms in real-time that can replace an entire Level-1 (or Level-0) trigger system in any of the large experiments at CERN-LHC
   DONE. A complete description of the 3D-Flow system for the Level-0 trigger for LHCb experiment was approved in less than one month from submission for publication by peer-review scientific journal.
18. Demonstrate feasibility in hardware of the concept of the 3D-Flow invention.
   DONE. The first demonstration of the 3D-Flow invention to be feasible and functional in hardware was presented at the 2001 IEEE-NSS-MIC industrial exhibition in San Diego (Calif.).
19. Demonstrate feasibility in hardware to build a 3D-Flow system for detectors of any dimension for HEP and Medical Imaging applications by developing modular electronic industrialized boards.
   DONE. In 2003, I proved the feasibility and functionality of 3D-Flow systems for HEP applications and Medical Imaging by building industrialized IBM PC modular electronic boards at my own expense, with 68 processors each. The successful testing of the communication between these two modular boards proved that a 3D-Flow system for any detector size in HEP or in Medical Imaging applications can be built, with the advantages of extracting all relevant information from radiation at the lowest cost per valid signal captured, to discover new particles, and to implement an effective, low-dose radiation, low-cost early cancer detection.
   NEEDED to advance science and for saving lives a fair PUBLIC merit review by funding agencies to fairly compare and fund the complete implementation of the 3D-Flow and 3D-CBS inventions capable of extracting ALL valuable information from radiation, found superior in terms of advancing science, saving money, and saving lives, so that humanity will no longer be deprived of the benefits from these inventions and taxpayers will no longer be burdened by funding less efficient and more costly approaches.

L. Scientific Innovations and Technical Merits of the Project

1. Example of the Sequence of Ideas, Followed by Analytical Thinking, Calculations, and Verification of Feasibility with Existing Technology which LED to One of the Many Inventions in this Project

The invention I have chosen as an example provides the most cost-effective approach to transfer 1.3 TB/sec data to the most compact, lowest power consumption, and most performant 3D-Flow system.

Step-by-Step Analytical Thinking which LED to My INVENTION of Transferring Data 28 TB/Sec @<$40K

Testable Up to 60 TB/Sec (480 Tbps) to Check when it Fails Between 2 ATCA or VXI Crates

This is just one example of one invention among several that I created for this project in order to optimize all aspects of the design that would provide the most powerful tool in discovering new particles and in the most effective device for early cancer detection, both benefitting from the advantages of extracting ALL valuable information from radiation by executing complex Object Pattern Real-Time Recognition Algorithms on thousands of data arriving in parallel from the detector at a million frames (or events) per second.

I followed a similar sequence of ideas, analytical thinking, calculations, etc., for the selection of the most cost-effective implementation of the 3D-Flow processor chip. In that case I had to evaluate which technology was most cost-effective (from 350 nanometer to 14 nanometer) for the volume of units (chip or ASICs, Application Specific Integrated Circuits) we anticipate needing, whether it was better to house 16, 64, or 256×3D-Flow processors in a chip, which I/O multiplexing rate was more advantageous for communicating with neighboring processors, and which was more cost-effective for Bottom to Top port of the processor, power consumption vs. speed and cost, etc. I am confident that all possible approaches have been considered and that the analytical thinking led to the choice of the most cost-effective approach of $1 per 3D-Flow processor for 55,500 chips with this price lowering to $0.42 per 3D-Flow processor for much larger quantities.

I also followed a similar sequence of ideas, analytical thinking, calculations, etc. in the selection of the most cost-effective FPGA to be used in my design of the LHC TER/Simulator (DSU) VME board. As you can see from the quotes, this process was able to identify the components for a board costing $3,000 using the most cost-effective FPGA having the same functionality as a board costing $18,000 with expensive FPGAs.

The variables I had to consider to find the best approach to transfer 10 Tbps from the PRAI crate to the 3D-Flow crate in FIG. 4 were many. Here is a list of a few:
1. Is it globally at the system level more cost effective to use 8,192 cables at 1.28 Gbps per cable or 1048 Ethernet cables at 10 Gbps per cable?
2. What is the component cost of each approach?
3. Can the dimensions of the connectors carrying the information from the 1024 cables in the 1.28 Gbps approach fit on the front panel of a 322 mm ATCA blade module?
4. Can the dimensions of the Ethernet connectors carrying the information from the 131 cables in the 10 Gbps approach fit on the front panel of a 322 mm ATCA blade module?
5. Considering the 1.28 Gbps approach, is it more cost-effective to use connectors with 0.8 mm pitch or 1.27 mm pitch?

I started answering these questions augmented by several others that arose in the process by first looking for a connector in the catalogue of Amphenol, Molex, Tyco and SamTec.

I found the SamTec series SEAM and SEAF and first selected the right angle 300-pins SEAF-30-01-S-10-2-RA-TR, with a total length of 49.28 mm. Four of these connectors just fit on the front panel of a 6U VME module 266.7 mm×20 mm and could be soldered on the PCB board 233 mm (4×49.28 mm=197.12 mm).

Next, I verified that this 300-pin connector carrying 131 LVDS signals would meet the speed requirements. Although the catalogue stated 36 Gbps, I asked the local SamTec representative and my question was copied to the regional representative, to the headquarter office in the U.S. and to the Signal Integrity (SIG) group at the manufacturing plant in Taiwan. The answer came back no, it would not support 1.28 Gbps because there were too few ground pins.

With a more in-depth study, I learned that to achieve 36 Gbps it was necessary to have only 75 differential lines and 150 ground pins with a pinout as reported in FIG. 51.

I studied in more in detail several other options from the catalogue and felt intuitively that stacking connectors with only 7 mm pin length might reduce the crosstalk that was coupling in the 15+mm pin length in the right angle connectors.

The engineer at the manufacturing company did not believe it would make much of a difference. I asked if he would do a simulation, and the next day I received an email from the same engineer in Taiwan stating “As you expected. 7 mm SEAM/SEAF crosstalk looks okay with only a few ground pins”, and provided graphs of the insertion loss and crosstalk at different frequencies showing the 7 mm stacking connectors working even when using fewer ground pins.

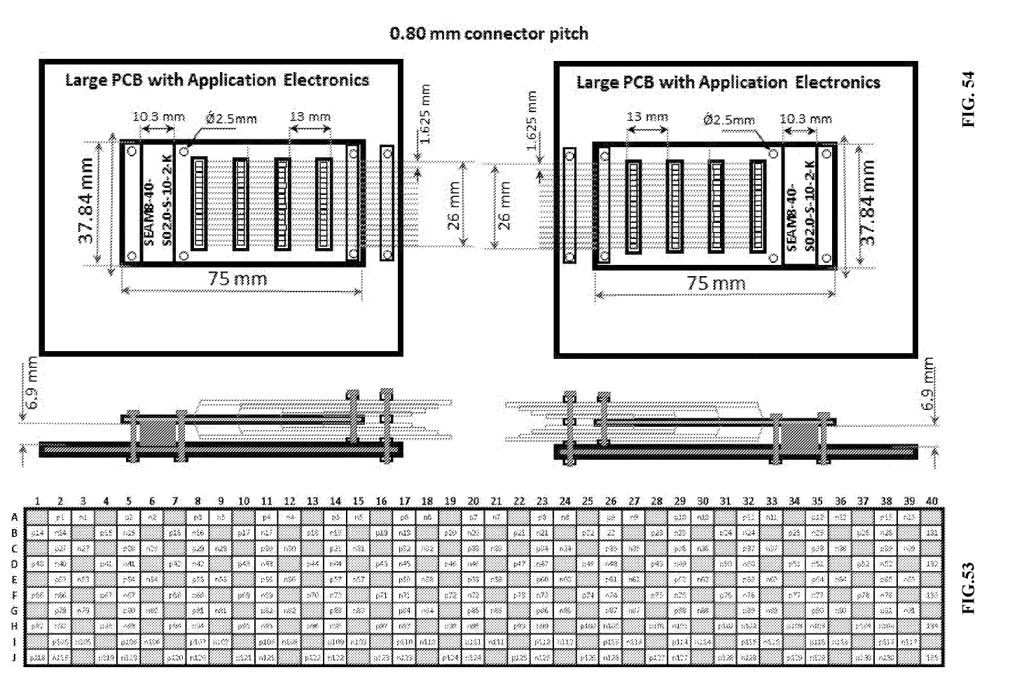

After learning the characteristics of these connectors that could achieve 36 Gbps and have the advantage of using 7 mm stacking connectors requiring fewer ground pins, I decided to build a system with the capability of 10 Gbps per Micro Twinax cable with a 50% safe margin of reliability in all components (I selected a connector with more pins and a cable that would support 10 Gbps). Therefore, I selected 400-pin stacking connectors SEAM-40-03.5-S-10-2-A for 131 LVDS signals (see FIG. 53) and I gladly accepted the compromise of good signal integrity in exchange for the necessity to extract the entire board to remove each connector instead of the ability to remove the connectors one at a time from the front panel of the board. I have used stacking connectors in the design of previous boards where one connector is on the PCB and the other on a FlexPCB where it was necessary to extract the board in order to remove the FlexPCB, and found it to be both reliable and easy to insert/extract the connector.

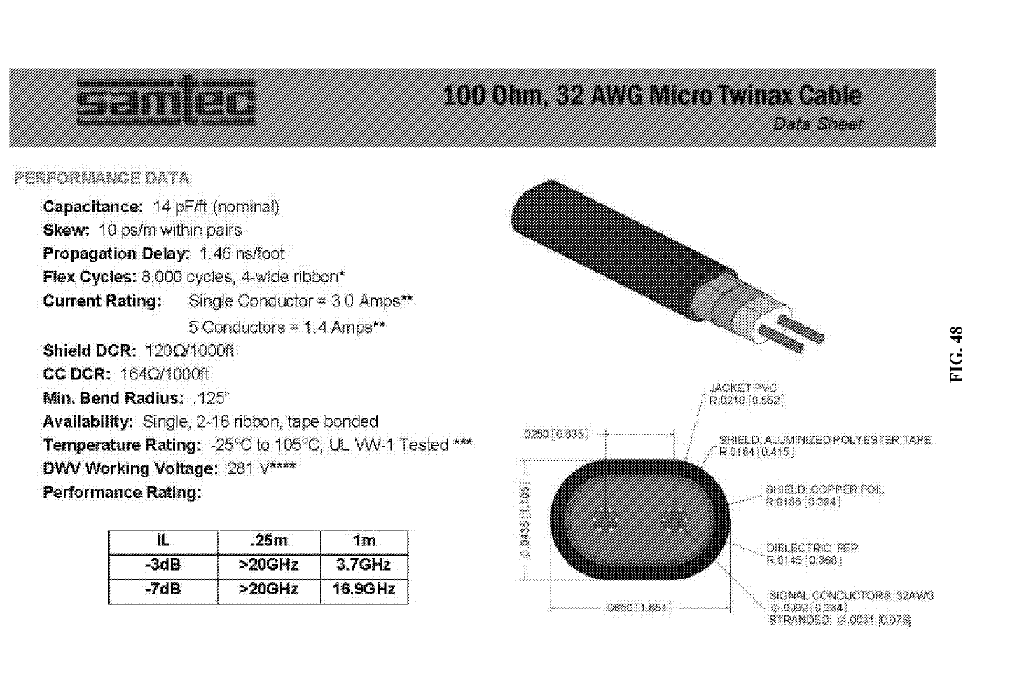

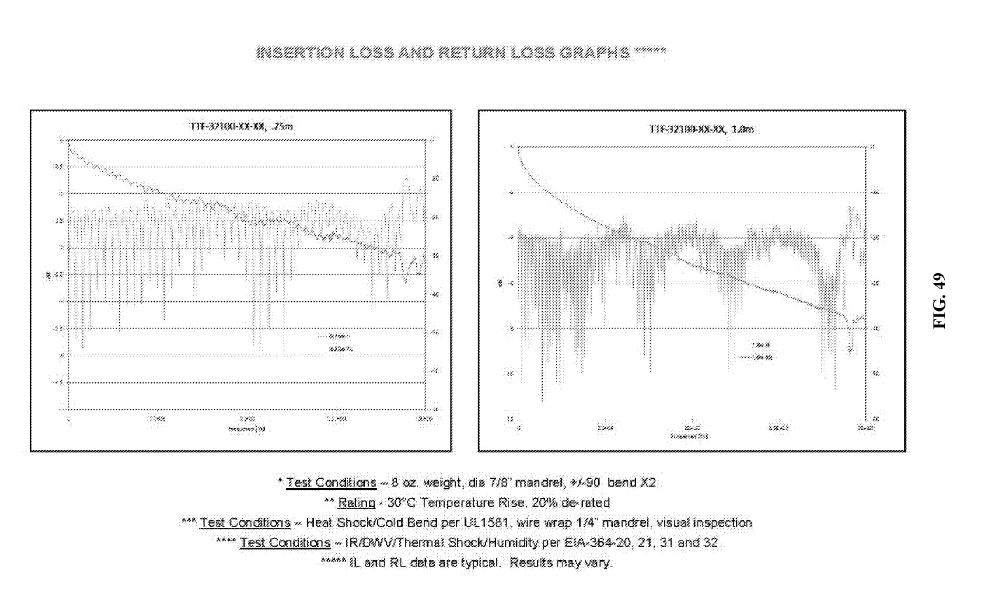

Next I needed to select the cable and made enquiries with a few companies on whether they could manufacture a FlexPCB connecting two SEAM-40-03.5-S-10-2-A connectors carrying 131 LVDS signals. Although none said it was impossible, there were some technical difficulties and I never received a quote. One of the companies that bid for the construction of the LHC TER/Simulator (DSU) board received a quote for a FlexPCB connecting the two 400-pins connectors, however it cost more than the solution I had come up with using Micro Twinax cables with the characteristics reported in FIG. (see http://suddendocs.samtec.com/notesandwhitepapers/ttf-32100-xx-xx datasheet pdf). See FIG. 48 and FIG. 49.

The Micro Twinax cable exceeds by over 10 times the speed needed in my application; it also has much lower insertion loss and crosstalk than needed. FIG. 49 shows the graph of the insertion loss at −3 dB for a cable length of 0.25 m working at a speed greater than 20 GHz and for 1 m working at a speed greater than 3.7 GHz. It follows that it could comfortably reach 10 GHz at 0.5 m length used in this application as shown in FIG. 4 (11U+2 inch=494.03 mm). Therefore, I am selecting the components (connectors and cables) targeting a system having the capability to transfer 10 Gbps per Micro Twinax with a margin that could reach a 50% higher frequency although I am using it only for 1.28 Gbps. This will provide a very reliable system.

I started communicating via phone and email with SamTec high speed cable application group in Costa Rica and sent the signal pin assignments of my design to SamTec connector SEAM-40-03.5-S-10-2-A as reported in FIG.

Considering the Twinax ribbon cable was made of 16×2 wires, I gave up the three control lines START, STOP and CLOCK from this connector and I planned to carry those signals on another connector.

On Nov. 19, 2015, I created a series of drawings, improving from one drawing to the next by thinking analytically, verifying the new drawings were satisfying all system requirements in the three applications described in FIG. 4, FIG. 5, and FIG. 6. Sometimes during verification I would notice some incompatibility or obstacle in some area, go back to the drawing board, modify my Power Point drawings, recheck, remodify, until all calculations, part dimensions, etc., harmoniously fit together.

This analytical process lead me in one day to invent a cable/connector assembly capable of transferring (with a margin of reliability of 50% higher speed), up to 28 TB/sec (or 224 Tbps) between two ATCA or VXI crates at a cost less than $40,000, which is exceeding all transfer rates used at CERN between two crates and it satisfies the requirements of CERN experiments for a few decades. For this specific application requiring the transfer of only 1.3 TB/sec between 8 electronics boards in one ATCA crate and 8 electronics boards in a VXI crate, the cost of the cables with connectors is approximately $10,000. It will also support future upgrades transferring up to 10 times this speed, being able to transfer up to 13 TB/sec without the need to purchase other cables. At the end of this section I will provide the calculations showing how this staggering transfer rate between two crates can be achieved.

I therefore created the first drawing for the assembly of the 128 Twinax cables connecting the two SamTec connectors at both ends of SEAM-40-03.5-S-10-2-A as reported in FIG. This assembly consists of two PCBs each soldered at one end to the connector; the other two ends of the PCB carries on each side two columns of two rows of 16×2 pads staggered 13 mm to avoid crosstalk between signals, and to leave room for UV epoxy covering the soldering of the wires of the Twinax ribbon cables.

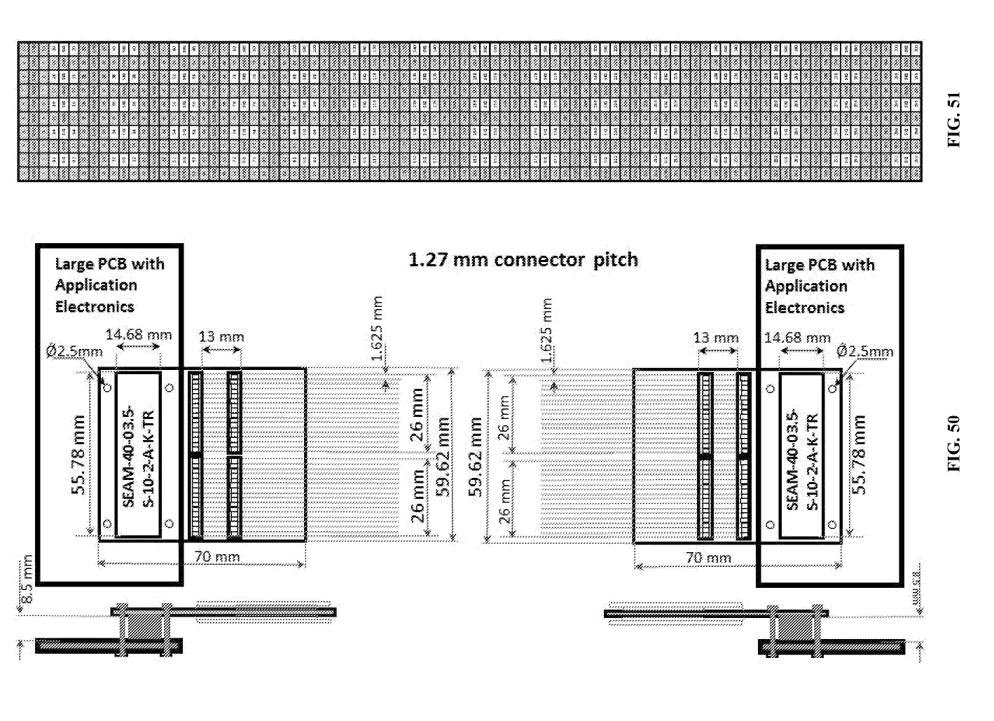

To provide high reliability during vibrations or if connectors become loose during insertion/extraction of the boards, I provided four screws at each corner of the connector. Unlike the larger holes for two screws at the center on both sides of the connector as commonly used in commercial products, I provided holes on the PCB for four smaller screws ( 3/32 inch) at the four corners of the connector drilled in the position that will keep the width of the PCB the same as the length of the connector. This will allow more connectors to be placed at the edge of the front panel of the application PCB. See FIG. 50.

I realized that I forgot to secure the ribbon cables to the small PCB to remove strain solicitations to the cable that could break the soldering of the wire to the PCB. However, I also realized that 56 mm width PCB was required because the length of the connector is 55.78 mm which will only allow 3 connectors to be placed at the edge of the VME board facing the front panel VME module (210 mm usable space), 5 connectors at the ATCA board (310 mm usable space), and 6 connectors at the VXI board (352 mm usable space). This requires more boards and more crates to house the same number of 3D-Flow processors, increasing the length of the cables which will increase the delay of the signals, which will increase its power consumption and cost.

An optimized modularity of LVDS channels per connector, connectors per board, boards per system is: 128 LVDS channels per connector, 2 to 4 connectors per VME board, 8 connectors per VXI or ATCA board, 8 or 16 VME boards per VME crate, and 8 boards per ATCA and VXI crate.

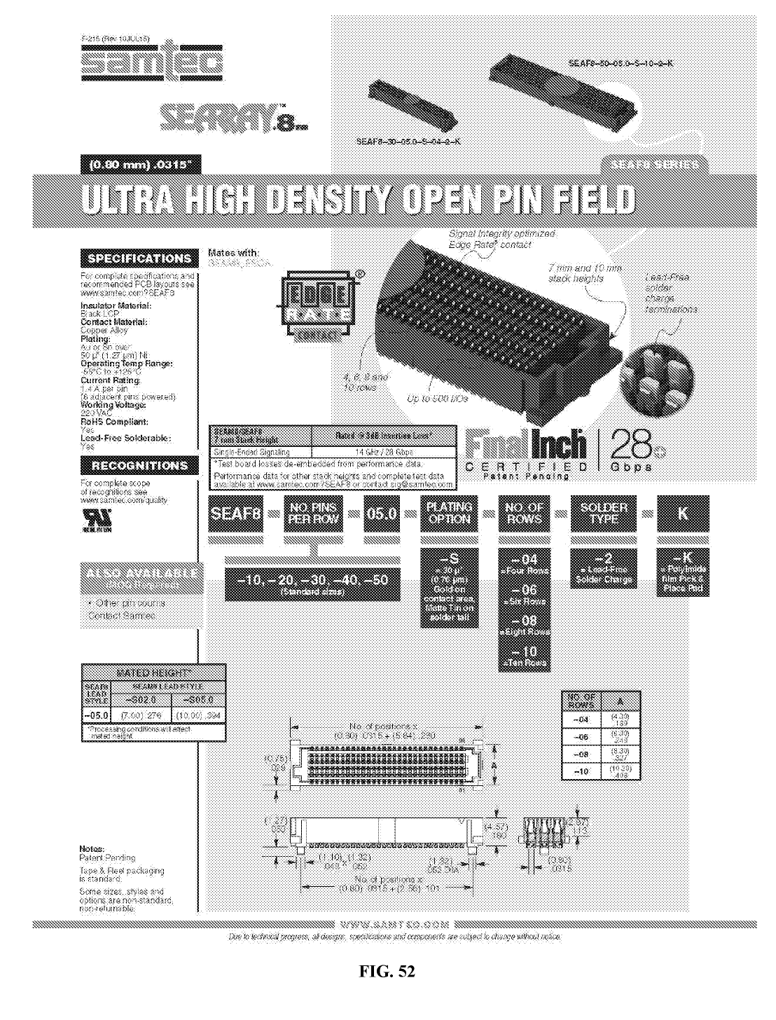

Upon further study of other options from the SamTec catalogue, I identified the series SEAF8, 0.8 mm pitch connectors with the specifications listed in FIG. that would provide 400-pin with a length 37.84 mm which would satisfy the requirements of placing the desired number of connectors per VME, VXI and ATCA boards listed before. See FIG. 52.

I went back to the drawing board of my Power Point and designed the layout of the small board 75 mm×37.84 mm housing the connectors SEAF8-SEAM8-40-S02.0-S-10-S-K, 0.80 mm pitch and the pads for 128 Micro Twinax Cables. The connector is assembled at one end of the board, while at the other end, on both sides of the board are four columns of pads. Each column has 16×2 pads for one ribbon cable. There are four ribbon cables on each side of the board. I then provided a strain reliever that keeps the eight ribbon cables tight together to the small 75 mm×37.84 mm boards and a second strain reliever tightening this bundle to the larger application PCB board. See FIG. 54.

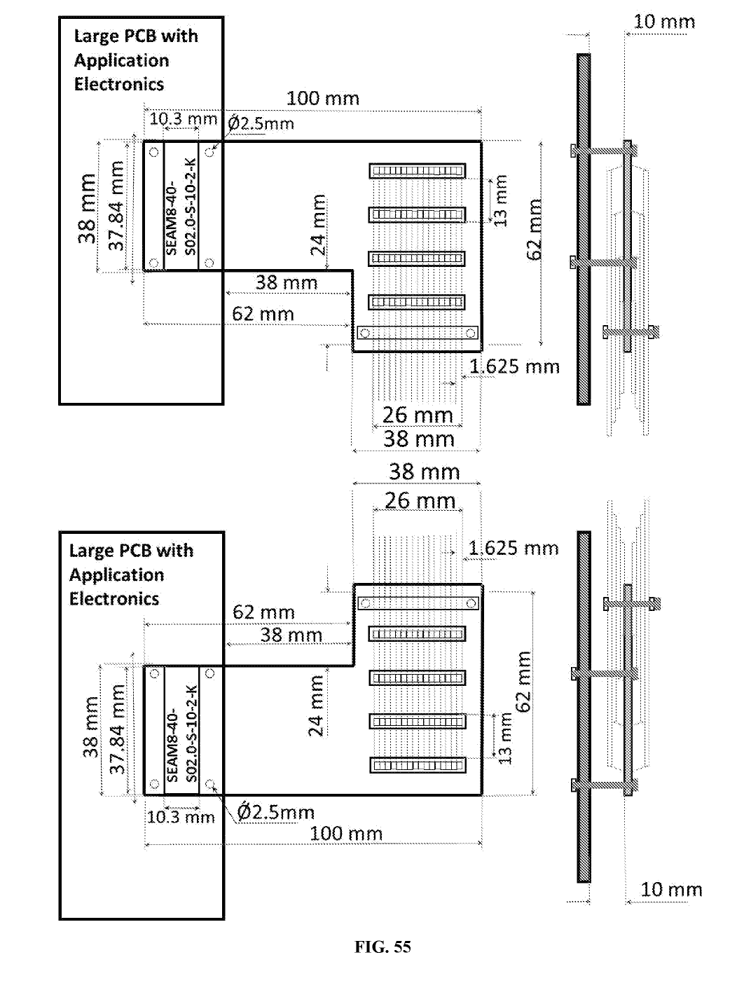

To improve utilization of the available space in the situation where several modules in one crate connect to modules in another crate, I gained space by orderly placing bundles of ribbon cables one next to the other and making the 90° cables turn on the small PCB as I drew in the next FIG. I provided a distance between the connector and the first ribbon cable of 38 mm to stagger two 128 Micro Twinax assembly to the larger PCB application board. The connector SEAF8-40-05.0-S-10-2-K of one assembly mates with connector SEAM8-40-S02.0-S-10-2-K at the larger PCB soldered at the edge, giving a stacking height of 7 mm, while another mates with connector SEAM8-40-S05.0-S-10-2-K, placed at 25 mm from the edge giving a stacking height of 10 mm. This will allow to increase the I/O to the board as also detailed in the caption of FIG. 55.

The assembly of FIG. 55 helps to better utilize the available space; it can be useful in some applications with a small number of boards, but the thickness of the bundle of ribbon cables of approximately 12 mm is still too high when more than one connector is needed to connect VME boards with an interconnector distance of 20 mm.

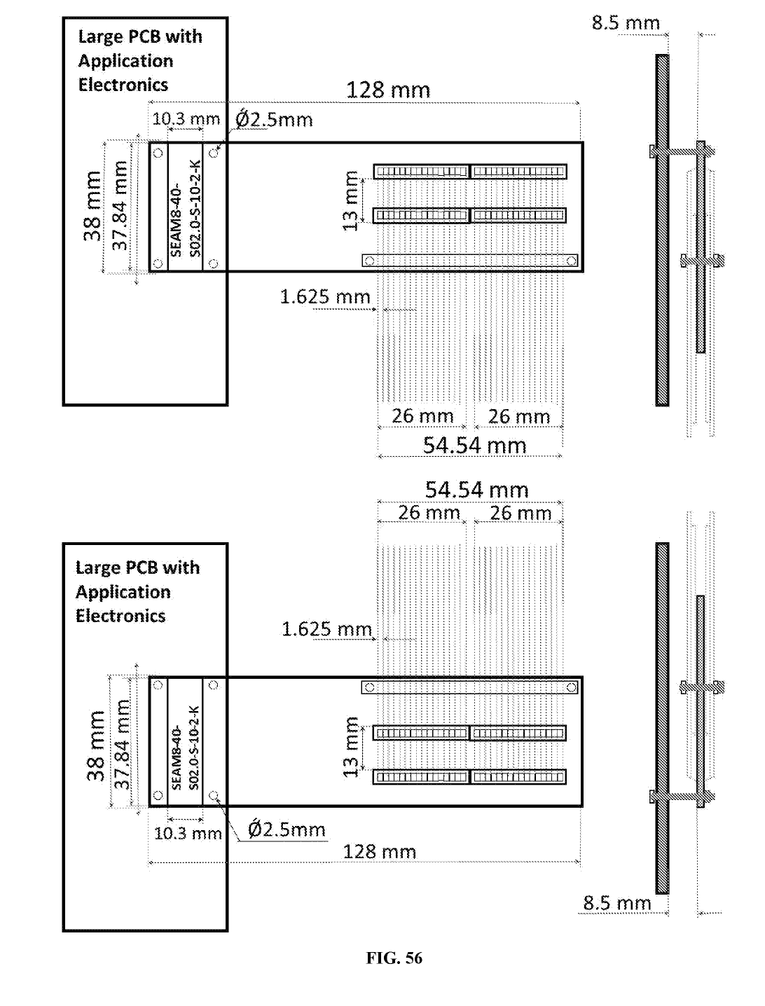

Therefore, I went back to the drawing board of my Power Point and created assembly of FIG. 56 placing side-by-side two ribbon cables on both sides of the small PCB board.

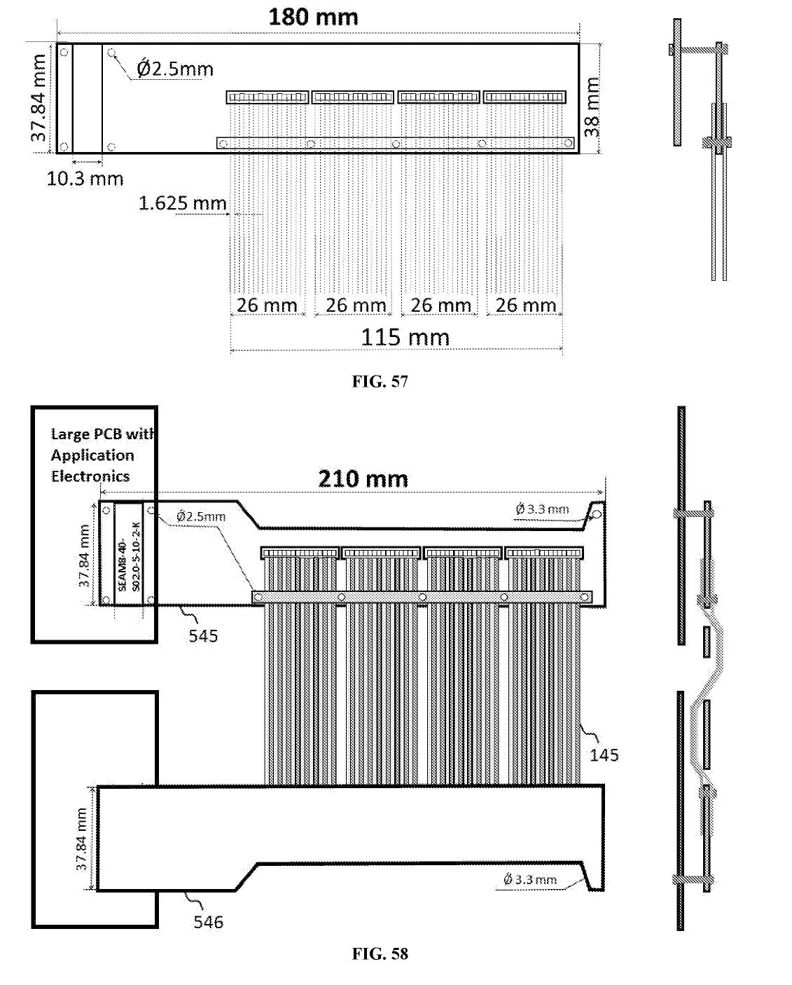

The cable assembly of FIG. 56 helped to solve the problem of the limited space to place ribbon cables between crates for some applications; however, placing four ribbon cables side by side and further reducing the thickness of the ribbon cables going from crate to crate as shown in FIG. 57, helps to solve more challenging problems requiring a high data transfer rate.

Further analytical verification of FIG. 57 made me realize that if a customer needs to place one of these little PCB boards next to the other because there is limited space on the front panel of the application board, then I should cut a piece of PCB along the width of the 4 ribbon cables as I did in FIG. 58 to create a window where the four ribbon cables can cross from one side of the PCB to the other side. I should also provide a hole at the corner of the small PCB board opposite to where the connector is assembled that will be used as shown in FIG. 6.

See also on the right of FIG. 58 how the ribbon cables are crossing over from one side of the boards to the other in order to keep an identical cable length.

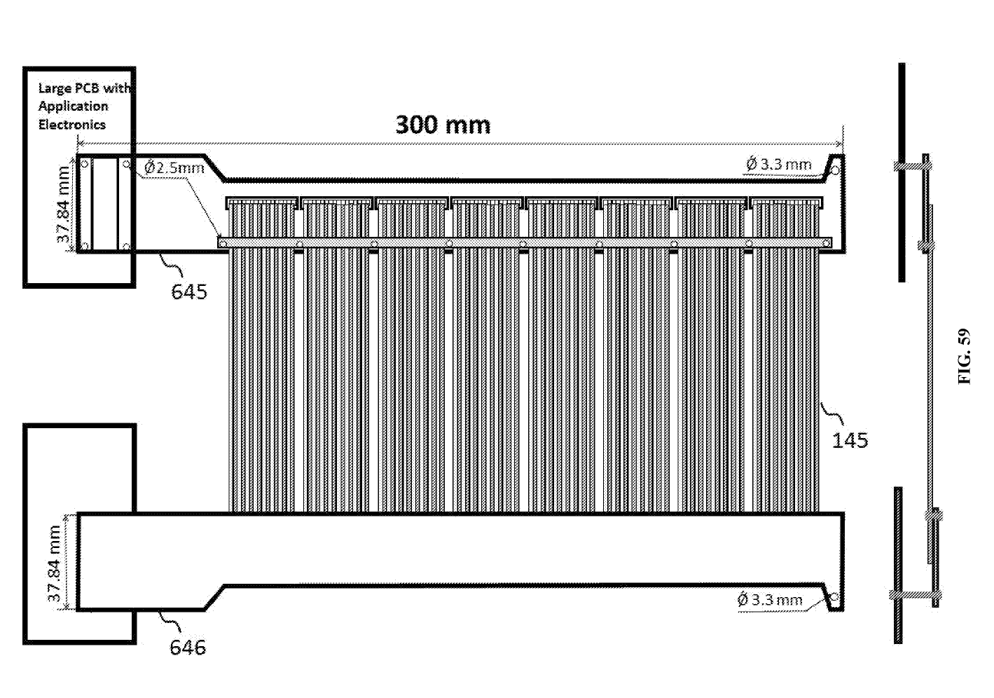

After a careful verification of the design of the assembly of FIG. 58, I concluded that it would be reliable for signal integrity, speed requirements and mechanical robustness. It satisfies the need to achieve a very reliable high data transfer rate between different boards located in different crates using up to four of these 400-pin connector-cable assemblies per VME board. It satisfies the need for the specific application of the LHC TER/Simulator (DSU) board with 512 channels requiring four of these cable assemblies per VME board. For more stringent requirements, where 8 to 16 of these 400-pin connector-cable assemblies are needed, or board-to-board space is narrow, I designed FIG. 59 assembly having only one layer of ribbon cables.

Lastly, I verified the difference in cost between this version of connector-cable assemblies using a 1.27 mm connector pitch as shown in and 0.8 mm connector pitch used in all subsequent drawings.

First I was told that the small PCB would cost much more because 1.27 mm pitch allows two signal traces in between, while 0.8 allows only one trace requiring an increase in the number of PCB layers to route all signals to the pads of the cable. Additionally, if the user's application PCB board having very simple circuits and components with pin pitch higher than 1.27 mm allowing the construction of a very economical PCB, the presence of a connector with 0.8 mm pitch will force the customer to add layers to the larger application PCB, increasing its cost.

This is not the case for this project because the ASIC with 64×3D-Flow processors used in the boards described in FIG. 4, FIG. 5 and FIG. 6 has a pitch of 0.8 mm, therefore the PCB board should have many layers.

However, it turns out the price of FIG. 50 cable assembly using 1.27 mm connector pitch compared to the cable assembly using 0.8 mm pitch, the difference is a nominal 8%.

At this point one cannot dismiss all previous drawings and state that only the last two of FIG. 58 and FIG. 59 are valuable because for specific applications a previous drawing and approach might be more cost-effective.

Likewise, I found it useful to report all analytical thinking in optimizing the design for a cost-effective connector-cable assembly application to achieve the highest transfer rate at the minimum cost, capable of speeds ten times the current transfer rate and satisfying requirements of future upgrade of the electronics. The considerations made in this analytical thinking might help optimize the connector-cable assembly to achieve cost-effectiveness in other applications, although I must say that it took less time to invent this cost-effective connector-cable assembly than writing this report.

One must be ready to modify the specifications as the work is progressing and feedback is received from those implementing it. For example, the need to cut the PCB on one side to create a window to let ribbon cables cross from one side of the PCB board to the other when there is limited space on the front panel of the application larger PCB as shown on the right side of FIG. 58. However, if the engineer who lays out the small PCB board for the version with eight ribbon cables placed side-by-side in FIG. 58 tells me he has difficulties routing equal length traces from the pads of the ribbon cable closest to the connector to the one further away because he does not have real estate PCB and is running out of number of layers he can add, I would agree to eliminate the cut which is shown to be not strictly necessary for the application of FIG. 4.

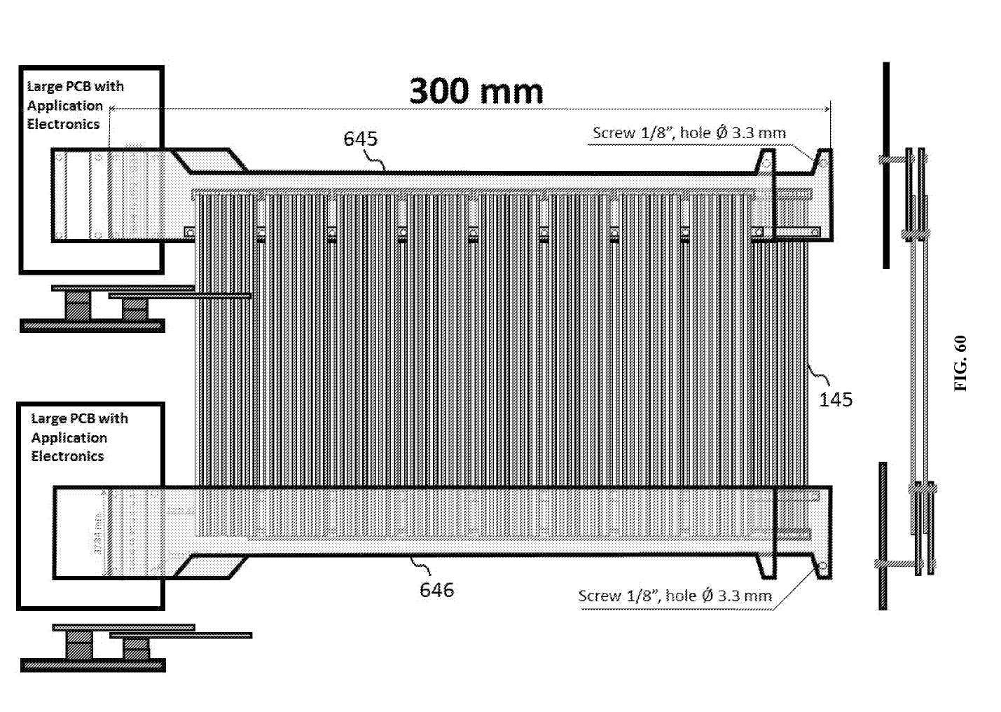

This assembly satisfies all needs for very large boards, crates and systems to achieve maximum transfer rate. It not only satisfies all system requirements in the three applications described in FIG. 4, FIG. 5 and FIG. 6 but exceeds them when staggered as show in FIG. 60 accommodate up to 16 of these 400-pin connector-cable assemblies in an ATCA board and up to 18 in a VXI board.

Using 64 of the 400-pin connector-cable assemblies of FIG. 59 to transfer data from 8,192 channels from the ATCA/PRAI crate of 4 to the VXI/3D-Flow crate will satisfy today's requirements of transferring 1.3 TB/sec, and without the need to change the cable assembly it can sustain future upgrades of the electronics at both ends up to 13 TB/sec.

For one entire application of a large experiment at CERN such as Atlas or CMS, the total cost of these connector-cable assemblies for 8,000 trigger channels is approximately $10,000. It would be a good investment as it will still satisfy upgrades which are 10 times faster at a transfer rate of up to 13 TB/sec.

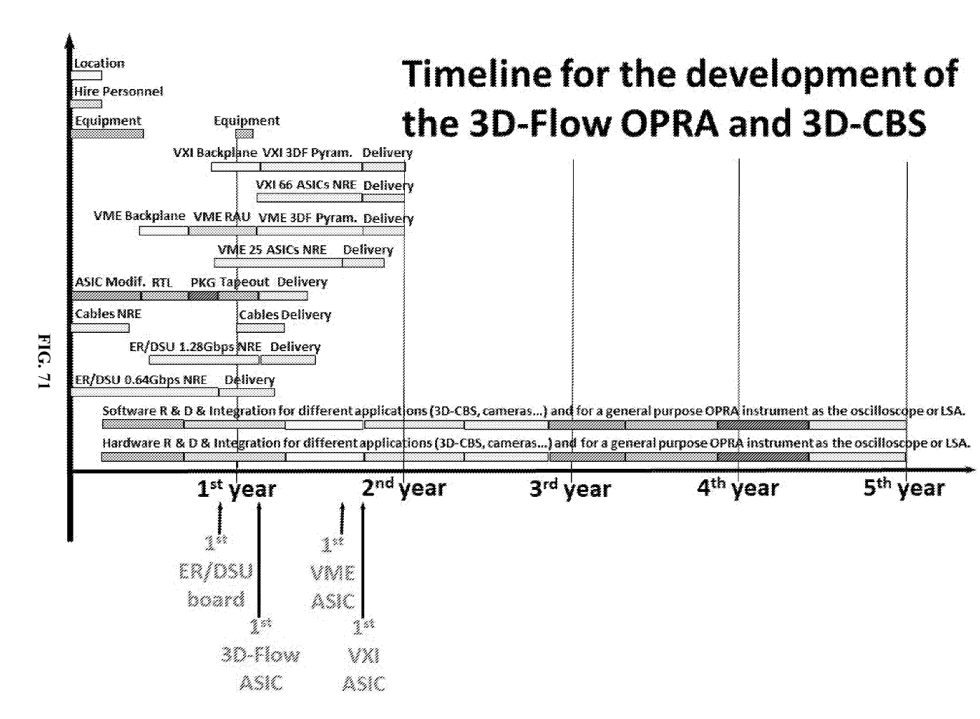

At this point we have much information that can help (with the help of catalogues from companies available on the web) answer the five questions asked at the beginning of this chapter. I report the answers in the same sequence they were listed at the beginning of this section.
1. At this time it is more cost-effective (and more reliable) to use 8,192 cables at 1.28 Gbps per cable instead of 1048 Ethernet cables at 10 Gbps, not just because of the lower price but more importantly because it provides a more powerful tool to execute complex Object Pattern Real-Time Recognition Algorithms with zero dead-time on 16-bit data arriving at 80 million events per second from over a billion collisions per second.
   Adding FPGA receivers to de-serialize the 10 Gbps to 1.28 Gbps that can be received by the 3D-Flow chip in the 3D-Flow board (either in the VME or VXI version) will increase the power consumption of the board.
   This will require distributing the same number of 3D-Flow chips across more boards because there is a limit the power each board can dissipate.
   Consequently, the size of the 3D-Flow system will increase, requiring longer cables for each chip in one board to communicate to its logical neighboring chip (in the global map of the detector array which is transferring data to the 3D-Flow processor array) on another board.
   A longer cable connection increases the time each 3D-Flow processor needs to exchange data with its neighbors to execute the Object Pattern Real-Time Recognition Algorithm, thus the number of layers of 3D-Flow processors needs to be increased to make a bigger system, requiring longer cables, further increasing the size of the electronics, and increasing the overall cost to achieve the same performance the 3D-Flow system would have obtained had it been contained in a smaller volume using shorter cables.
   To optimize performance and cost I need to keep the smallest possible number of extra components in the 3D-Flow board that are not the 3D-Flow chips.
   Using 10 Gbps data rate to the 3D-Flow boards if the 3D-Flow chip could receive directly 10 Gbps input was considered, but an informal enquiry at the ASIC design house, revealed the NRE would be over four times as expensive. This issue could be discussed with the funding agency. There is no preclusion from my side to go against CERN's standardization of 10 Gbps, I am just providing the cost-performance data and I would be glad to comply for the higher speed. In any event, the cables proposed from this study would satisfy the requirements of both the 1.28 Gbps and the 10 Gbps approach.
2. The component cost of the 8,192 cables at 1.28 Gbps is $10,000.
   The component cost of 1048 Ethernet cables at 10 Gbps is over $120,000 calculated as follows:
   The cost to assemble one cable with two Ethernet connectors is $41 (see Amphenol website for price), ×1024=$41,984, plus the cost of approximately $40,000 for the transmitter FPGAs serializing the bits from 1.28 Gbps to 10 Gbps and $40,000 for the receiver FPGA de-serializing from 10 Gbps to 1.28 Gbps (See DigiKey website for FPGA prices).
   Using the FPGA approach of serializing and de-serializing will waste a lot of money because these FPGA chips combine transmitters and receivers in the same chip, therefore at the transmitter board the receivers' functionality will be wasted and vice versa. I found the answer I received from the clerk at a computer store in Dallas interesting when I asked whether they had any unidirectional Ethernet boards. His answer was no as they are targeted only to people who communicate in both directions.
   In our application, instead we have the need to transfer data at 1.3 TB/sec in one direction and a few 1 KB/sec in the other direction (see FIG. 2), therefore we cannot copy the world of electronics form (ATCA, VPX, etc.) developed for telecommunication. Although in both fields, HEP and telecommunication, we have a need for a high data transfer rate, HEP is different because it is mainly unidirectional.
3. FIG. 58, FIG. 59, detail how eight SamTec SEAM8-40-S02.0-S-10-2-K connectors 0.8 mm pitch, 37.84 mm in length carrying 1024 LVSD differential signals fit on the front panel of a 322 mm ATCA blade (with a usable space of 310 mm), while FIG. 70 details how 2048 LVDS differential signals can be received by an ATCA board using a column of connectors at the edge of the board SEAM8-40-S02.0-S-10-2-K providing a stacking height of 7.0 mm, and at 25 mm from the edge of the ATCA board a column of connector SEAM8-40-S05.0-S-10-2-K, which provide a stacking height of 10 mm which would allow two connector-cable assemblies to be staggered.
4. Because the dimensions of the Ethernet connector cannot fit 128 connectors on one ATCA blade or VXI board, the electronics will increase the number of boards and number of crates needed, increasing the overall cost. (See dimension of the 10 Gbps Ethernet connector at http://wwe.cablesondemand.com/category/SFP+%20CBL/product/SF-SFPP2EPASS/URvars/Items/Library/InfoManage/.htm?gclid=CNLe8JPZuMkCFQIHaQodo_8D qA)
5. There is only an 8% increase in cost to have the 0.8 mm pitch connector instead of the 1.27 mm pitch connector solution as reported in quote HDR-190421-01 on page 133 of BJ. However, the 0.8 mm pitch connector has many technical advantages including increased system performance because it allows four 0.8 mm pitch connectors to be placed on the front panel of a VME board and 8 connectors in the VXI and ATCA boards. This will reduce the size of the system and the power consumption and increase the performance while greatly reducing the overall cost.

In summary, to have the best price of the connector-cable assembly it is necessary to maximize the use of one type of connector-cable assembly to reach the rock-bottom price for 500 units.

What will drive the universities and laboratories to order one type or the other is which form factor has been chosen. This will depend on whether the funding agencies will fund more electronics using large boards such as VXI and ATCA or smaller boards such as VME.
a. Implementing FIG. 59 with eight Micro Twinax 16×2 ribbon cables assembled all on one side of the small PCB is best for the larger VXI and ATCA boards. This assembly requires a deeper mounting frame occupying more space in front of the crate to support the weight of the cables as shown in FIG. 4. The maximum performance of this connector-cable assembly used at 1.28 Gbps per channel for the full use of the 12 slots of a VXI crate with 8 connectors per board would be 15.7 Tbps (calculated as 1024×1.28 Gbps×12=15.7 Tbps), or when used at 10 Gbps, the overall performance would be 122.8 Tbps (calculated as 1024×10 Gbps×12=122.8 Tbps). The price of all connector-cable assemblies will be the same—approximately $16,320.
6. Implementing FIG. 58 with four Micro Twinax 16×2 ribbon cables assembled on both sides of the small PCB is be the best for the smaller VME, VPX boards. This assembly requires a smaller mounting frame occupying less space in front of the crate to support the weight of the cables as shown in FIG. 6. The maximum performance of this connector-cable assembly used at 1.28 Gbps per channel for the full use of the 20 slots of a VME crate with 4 connectors per board will be 13.1 Tbps (calculated as 512×1.28 Gbps×20=13.1 Tbps), or when used at 10 Gbps, the overall performance will be 102.4 Tbps (calculated as 512×10 Gbps×20=102.4 Tbps). The price of all connector-cable assemblies will be the same—approximately $13,600.

The estimated performance and cost when using FIG. 60, connector cable assembly, taking advantage of its full transfer rate capability between ATCA, VXI and VME crates, staggering two connectors, one at the edge and another 25 mm from the edge of the board, with the pinout recommended by SamTec (FIG.) is as follows:
a. The maximum data transfer rate achievable between 2 ATCA crates with 14 slots assuming 16 connector-cable assemblies per board, and each channel having a transfer rate of 10 Gbps will be 28 TB/sec (or 224 Tbps). This is a very conservative estimate because SamTec catalogue provides the performance of these connectors at 22 Gbps and 0.5 m Twinax can provide >11 MHz at −3 dB insertion loss. In fact the transfer rate can be tested from 28 TB/sec up to 49 TB/sec to see when it fails. The price of all connector-cable assemblies will be approximately 538,000.
b. The maximum data transfer rate achievable between 2 VXI crates with 13 slots assuming 18 connector-cable assemblies per board, and each channel having a transfer rate of 10 Gbps will be 29 TB/sec (or 234 Tbps). This is a very conservative estimate because SamTec catalogue provides the performance of these connectors at 22 Gbps and 0.5 m Twinax can provide >11 MHz at −3 dB insertion loss. In fact the transfer rate can be tested from 29 TB/sec up to 51 TB/sec to see when it fails. The price of all connector-cable assemblies will be approximately $39,000.
c. The maximum data transfer rate achievable between 2 VME crates with 21 slots assuming 8 connector-cable assemblies per board, and each channel having a transfer rate of 10 Gbps will be 21 TB/sec (or 168 Tbps). This is a very conservative estimate because SamTec catalogue provides the performance of these connectors at 22 Gbps and 0.5 m Twinax can provide >11 MHz at −3 dB insertion loss. In fact the transfer rate can be tested from 21 TB/sec up to 37 TB/sec to see when it fails. The price of all connector-cable assemblies will be approximately $28,000.

2. Likelihood of Achieving Valuable Results

Because the 3D-Flow invention has been proven to be feasible and functional in hardware FPGA it can be stated that results can be achieved.

Simulation of large 3D-Flow system in C++ down to the RTL level with VHDL and synthesized in standard cell of a 350 nanometer technology is an additional proof that valuable results can be achieved.

Errors in developing the electronic boards and ASIC can only delay the results in requiring two to three spins (or versions) of the prototypes, however, my past experience in developing very complex boards with a high density of components in a small PCB area such as the FDPP board and the 3D-Flow board with 68×3D-Flow processors and successfully working at the first version produced makes me feel confident that the 3D-Flow system (boards and chip) with the new ASIC with 64×3D-Flop processors per chip would work at the first version built or in worst case at the second version.

3. How Results of the Proposed Work Might Impact the Direction, Progress, and Thinking in Relent Scientific Fields of Research

Data from the World Health Organization show that cancer as the most deadly premature (less than 70 years of age) calamity. National Geographic shows that we were aware since 1946 that Early Detection can save over 50% of lives. U.S. NIH-NCI-SEER (Surveillance Epidemiology End Results) shows experimental data that when cancer is detected early, 49.5% of lung cancer patients survive . . . 100% of prostate cancer patients survive, but when detected late only 2.8% of lung cancer and 31% of prostate cancer patients survive (right column). The ultra-sensitive 3D-CBS can provide an effective Early Cancer Detection, saving lives.

Doctors and journalists who claim that screening can cause unnecessary risky procedures should work to identify the incompetent doctors who diagnose false positives and help to improve training of medical personnel. When an astronaut or engineer at NASA makes a mistake and a Shuttle crashes we do not abolish NASA. When a pilot makes a mistake, journalists not write articles to ban air transportation. When drivers make an error and cause an accident, journalists do not write articles to ban motor vehicles traveling faster than 20 mph but we all help to improve car safety and better train drivers. We cannot stop advancement in science that will provide more accurate medical instrumentation to doctors because they do not know how to use their information; we should all help to better train doctors, so they will avoid to make mistakes of false positives.

4. How the Proposed Project Compares to Other Efforts in the Field, in Terms of Scientific and Technical Merits and Originality

a. In HEP applications it replaces many crates of electronics with a single crate of electronics containing a much more powerful system

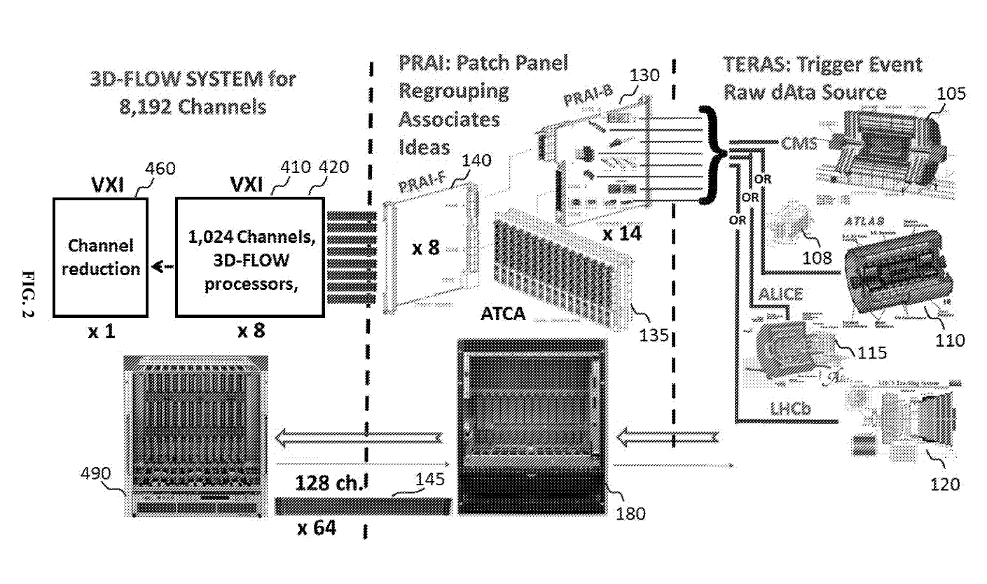

I have summarized in one picture (See FIG. 3) the importance to address this invention more in-depth.

b. In Medical Imaging applications the 3D-CBS is hundreds of times more efficient than current PET and PET/CT enabling early cancer detection, using a low radiation dose at an affordable examination cost. It has shown to be superior at the public review during the Leonardo da Vinci competition

M. Appropriateness of the Proposed Approach

1. How the Research Approaches are Logical and Feasible

I started my investigation in this field of applications by typing some key words into the Google search Engine such as “Trigger HEP”, “Trigger LHC experiments” “Trigger CMS”, “rigger Atlas”, etc. and among the first on the list was an article by Wesley Smith (2013), one by Philipp Grobs (2014) and several others.

I read both and several others. Wesley Smith's article http://arxiv.org/ftp/arxiv/papers/1307/1307.0706.pdf after the first chapter “Findings” reporting the requirements (some requirements are also found in the Chapter “Executive Summary”), which are also found in several CERN documents, I read some very confusing statements in the Chapter “Recommendations” and “Executive Summary”.

By listing all components, technologies, form factors, CPUs, protocols, links at different speeds, making a big salad without describing what is important for this application in processing power, in link speed vs. price, etc., it clearly reveals that the author does not have a clue which analytical path to follow to find the most cost effective solution to capture and accurately measure the characteristics of new particles (objects).

An analytical thinker would start by looking at the number of channels from different subdetectors participating to Level-1 Trigger (calorimeter, tracker, muon . . . , etc.), the timing relation between them, and whether those with a larger number of channels can be funneled to 8,000 channels of the calorimeter Trigger Tower. They should then ask themselves how many bits are necessary for each subdetector Trigger Tower or generally speaking, for all detectors within a certain view angle, and what is the total size of the words per channel needing to be transferred to the Trigger Processor every 25 ns; is 16-bit sufficient, or is 32-bit or 64-bit necessary? What kind of operation is necessary to perform on those bits or group of bits? Do we need to exchange data with neighboring processors to execute object pattern recognition? How efficient is FPGA to perform these operations?

Instead, in Chapter 2 and Chapter 3, Smith lists FPGA, ATCA, Micro-TCA, Advanced Mezzanine Cards standard, GPUs, ARM, Xeon Phi, TCA for backplanes GbE, SATA/SAS, PCIe, SRIO, High Performance Computing (HPC) interconnect, Network Interface Cards (NICs), 40 Gbps, 100 Gbps, 100 Tbps which should be achievable with today's switches at 32 Tbps . . . Why? If I can transfer 234 Tbps across 23,400 Twinax cables between two VXI crates with $39,000, as detailed on page 57, why do we need to use expensive telecommunication equipment designed to communicate in both directions when we need only to transfer data in one direction? Perhaps to complete the panorama of the long list mentioned before we can also add Hypercube. However, which one of all those components listed by Smith finds the desired particle more cost-effectively?

I am not surprised to read these statements by Smith. I have been trying to help him follow analytical thinking since 1992 when he was in charge of the Trigger of the SDC experiment at the Superconducting Super Collider, but when I invented the 3D-Flow architecture offering programmable object pattern recognition at Level-1 Trigger, he was one of a handful of people who stated that “we do not need programmability at Level-1 trigger”. I have been trying to help him understand for many years. In 1998, I attended a workshop on the electronics for LHC at Snowmass (CO), and experiencing a closed door from him as Co-Chairman and Co-chairman Peter Sharp, who would not let me present my invention and my analytical thinking. The last time I paid him a visit was in his office at CERN in 2008 when he recanted many statements and positions he had taken before; however, he continues to be closed to analytical thinking, following instead his original ideas, which at the time of the Super Collider was his cluster finding ASIC, protecting his own idea instead of openly comparing its effectiveness with other approaches, and supporting and funding the author with the better idea.

His approach precludes the birth and raising of innovative ideas; he does not see how his approach which crushes analytical thinking is detrimental to the scientific community and to taxpayers; he does not see the evidence of the failure of the Level-1 Trigger that could not and cannot identify particles with the characteristics defined by experimenters. Yet it is clear from the results of experiments that could not find even a small percentage of the 100,000 Higgs boson-like particles predicted to be present, and from the admission by CMS collaboration that Level-1 Trigger must be trashed and a new one built, that the current Level-1 Trigger is a failure.

Luckily the LHC collider worked, producing particles at a higher energy than the Tevatron at FERMILab, allowing us to go one step further in the understanding of our universe with a more powerful tool, and despite the trigger being ineffective, the data acquisition of the LHC experiments was able to casually record some events different than others. Now, however, it is necessary to build a very effective trigger that can nail down the nature of these Higgs boson-like particles. As I mentioned in one of my previous documents, the Nobel Prize should have been assigned to those who built the LHC collider working at an energy level never before achieved.

Ignoring, refusing, boycotting or blocking open public forums which would exercise analytical thinking should not be allowed because it is damaging the scientific community and the public who trust scientific procedures are being followed. The decisions of one person with funding power but who is not supporting the best approach, as in the case of Wesley Smith, is driving into the ditch thousands of scientists who worked for 20 years to develop software and hardware to analyze noise data because Level-1 Trigger did not work, as it happened for CMS and the collaboration decided to trash it. The same thing happened for Atlas who also decided to trash their Level-1 Trigger.

Wesley Smith is not the only scientist unwilling to have an analytical discussion where everyone would gain. For example, over the years I have tried having an analytical discussion on the Level-1 Trigger with Patrick LeDu, a senior organizer of the IEEE-NSS conferences. Our relation has always been cordial; however, the typical course of events at the IEEE conferences is on day one I ask him for a meeting; he schedules our meeting during a coffee break but then never shows up; we set another meeting, and again he does not show up. He never answers email on technical-scientific issues. When he has spoken a few words to me, it is to lament that the government does give him enough funds to keep the bright students at the university after graduation, or that he had some bright idea like mine for the trigger; but he never wants to discuss and compare his idea with my invention, providing the excuse that he is very busy attending several meetings to plan the future.

At the 2013 conference in Seoul, I noticed that he was the convener at the Trigger Session. Hoping to be able to address analytically trigger issues, I went to the session and asked the presenters questions. They clearly stated that Level-1 Trigger was not fully programmable nor did it operate with zero dead-time. LeDu did not chair his session because he was attending meetings to plan the future which he apparently believed is more important than discussing analytically the essence of the future in HEP with his colleagues, presenting different trigger ideas and hearing comments from other participants.

Another leader, Ugo Amaldi, told me before the keynote speaker's lecture at the 2012 International Conference on Translational Research in Radiation Oncology in Geneva, not to ask the speaker any questions and not to contact newspapers. This surprised me as I believed a renowned physicist like Amaldi would be eager to hear an exchange of questions and answers and to share new inventions with the public, just as the Chairman of the ICATPP conference (Pier Giorgio Rancoita) had in 2005 when he called for a Press Conference to announce my 3D-Flow and 3D-CBS inventions that create a revolution in the field.

Or Amaldi could have organized a public debate where physicists that he knows could challenge my analytical thinking by asking questions; the outcome would only help to advance science. Others who denied analytical, scientific discussion and transparency in science are: Ingrid-Maria Gregor, Chair, and Adam Bernstein, Deputy Chair, of the 2014 IEEE-NSS (Nuclear Science Symposium Conference), who denied analytical discussion and transparency in science when they rejected my proposed workshop on transparency (see details at: http://blog.u2ec.org/wordpress/?p:=150); Anthony Lavietes, General Chairman of the 2014 IEEE-NSS-MIC-RTSD conference, interrupted my legitimate question to the 2014 IEEE-NSS Keynote speaker, provided a statement inconsistent with science, and then walked away.

In the field of Medical Imaging, Alberto del Guerra is considered one of the leaders. However, I have never been able to discuss analytically his review of my book, or why his reasoning that we do not need a PET device with a FOV longer than the size of the largest organ (16 cm) is flawed. He has been invited several times by organizers of workshops on my 3D-CBS technology from the University and San Matteo hospital in Pavia to be a member of the panel of experts or reviewers to publicly express his evaluation regarding my invention which would allow me the opportunity to answer his questions, doubts and/or concerns, but has never accepted. I would also like to discuss with him the rejection of my papers, but he repeats they were rejected for their low score in scientific merit without wanting to discuss what they consider as “scientific merits”. (See his latest rejection based on a score that he cannot support http://blog.u2ec.org/wordpress/?p=1363). Others who denied analytical, scientific discussion and transparency in science regarding medical imaging are: George Alfakhri & Katia Parodi, Chair and Deputy Chair of the 2014 IEEE-MIC (Medical Imaging Conference), who denied analytical discussion and transparency in science by rejecting a workshop (see details at: http://blog.u2ec.org/wordpress/?p=956); Craig Levin, Deputy Chair of the 2013 IEEE-MIC conference, who built and published PET with very low sensitivity and high spatial resolution but rejected my papers for a very high sensitivity and high spatial resolution.

When I had to choose the form factor to implement the ER/DSU and the 3D-Flow ASIC boards, I was attracted at first by VPX in place of VME because I read articles that CERN was moving toward VPX standard and I did not want to stay behind. I had similar considerations toward ATCA compared to VXI; however, a deeper investigation made me realize that there are no advantages, and in fact it costs more.

The Telecommunication Industry is doing an excellent job defining new standards such as ATCA, VPX, Micro-ATCA, etc. However, HEP's needs are different. It needs unidirectional communication, and because SERDES Ethernet, cables, components, connectors, are bi-directional, either a transmitter is going to waste on one side or a receiver on the other side. In addition, the price of VME connectors compared to VPX connectors is considerably lower.

I asked colleagues and professionals in the industry to point out the differences or advantages of VPX compared to VME. I asked professionals to provide names of people at CERN who were advocates of VPX, and was directed to read this article http://iopsscience.iop.org/article/10.1088/1748-0221/10/01/C01008/pdf. The article did not provide a comparison in price or features that showed great advantages for CERN to use VPX instead of VME; in fact, for my application I found advantageous in using VME.

We should focus on the objective of creating very powerful tools that allow experimenters to discover new particles at the lowest cost per experiment. We should be free to choose the more cost-effective form factor and transfer speed: ATCA, VXI, VME, VPX, 1.28 Gbps, 10 Gbps, 40 Gbps, 100 Gbps, etc. in each specific application as I did in this proposal. I used ATCA form factor for the PRAI crate (Patch-Panel Regrouping Associates Ideas), VME for small 3D-Flow systems, and VXI for large systems. Today it is more cost-effective to use 8,192 Twinax cables at 1.28 Gbps (up to 10 Gbps) to reduce power consumption in the 3D-Flow boards that do not need a de-serializer because their input port speed is 1.28 Gbps. In the future, when it becomes cost-effective to build a 3D-Flow chip with Top and Bottom port I/O speed at 10 Gbps, then using 10 Gbps all the way from the detector to the 3D-Flow electronics will be most cost-effective, and eliminate the cost of serializers and de-serializers.

N. Significant Potential Problems and Alternative Strategies to Resolve them

Potential problems are not underestimated and are anticipated as well is anticipated their solution.

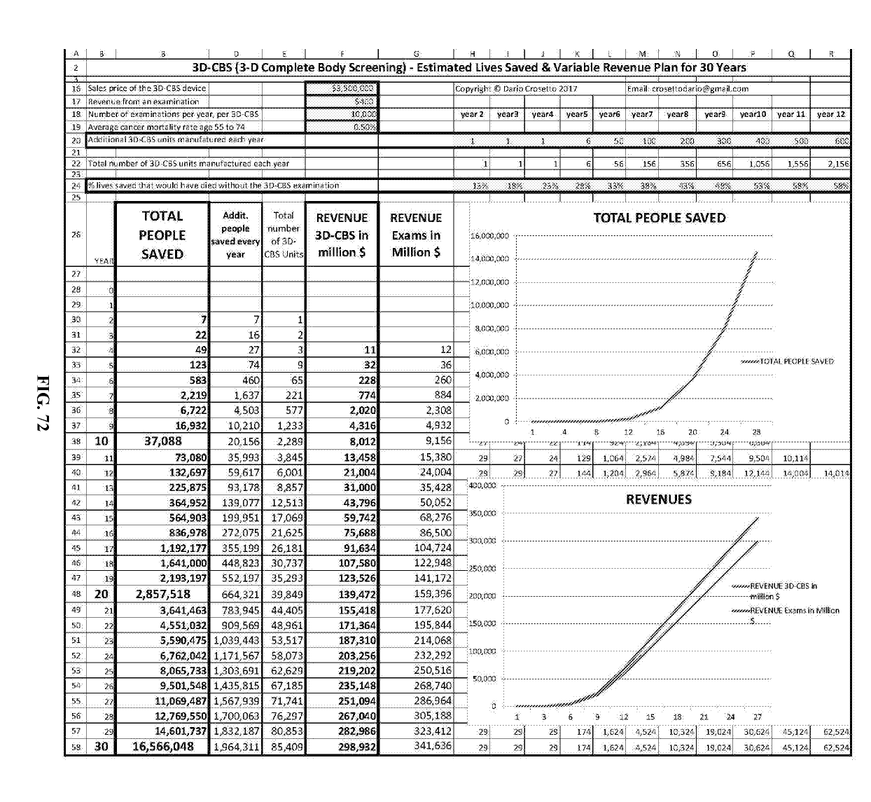

As you can see in the Timeline for the development of the 3D-Flow and 3D-CBS systems of FIG. 71, I have planned the development of the VME 25 ASICs and VXI 68 ASICSs after the final calculation of the power consumption of the 3D-Flow ASIC with 64×3D-Flow processors. In the event it is greater than 4 Watt the design of the boards will be modified to accommodate fewer ASICs to be able to handle boards and crate power dissipation.

Instead for the possible failure of connections on connectors or cables or failures on some processors, I had already developed with the previous DOE $906,000 grant in 1997 a fault tolerant program that is identifying the faulty cable, connector, ASIC or component and is isolating the offending components permitting to continue data acquisition with one or a few dead-channels.

### The 3D-Flow System is Fault Tolerant

During operation the entire 3D-Flow system made of thousands of 3D-Flow processors is monitored in real-time through the USB port at each chip that is accessing the status bits of each of the 64×3D-Flow processors in the chip.

Fault tolerant programs have been already developed that can detect broken processors or cables.

When a problem is detected, such as a non-responsive processor or a broken cable, this fault-tolerant monitoring program loads a modified program into each neighboring processor where there is a broken cable or broken processor.

This will allow the system to continue operation with one dead channel out of 8000 channels until the next shutdown of the data acquisition system when the maintenance team can access and repair the faulty component or cable.

This fault tolerant monitoring program also saves time diagnosing the problem because it can point out the chip ID or location of the broken cable.

O. Examples of the 3D-CBS Detector Implementation

The 3D-Flow OPRA has the capability to extract and optimize cost-effectively the measuring of all characteristics of the sought objects analyzing signals from detectors of different shape (circular, elliptical, etc.) and of different types (different crystals such as LSO, BGO, LYSO, solid state detectors, etc.) from detectors of the 3D-CBS (3-D Complete Body Screening).

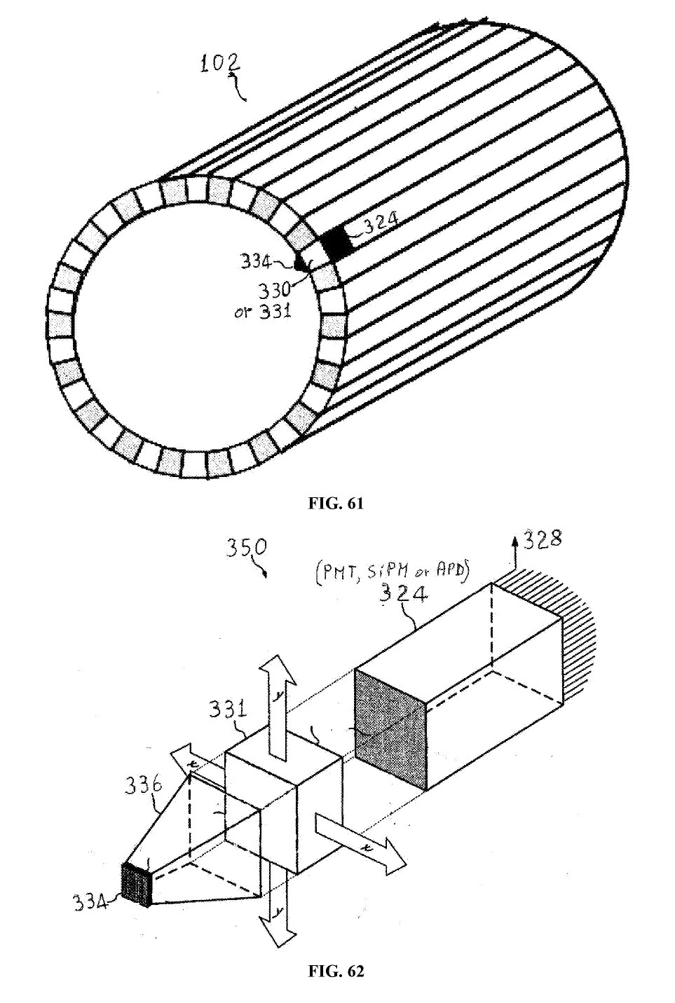

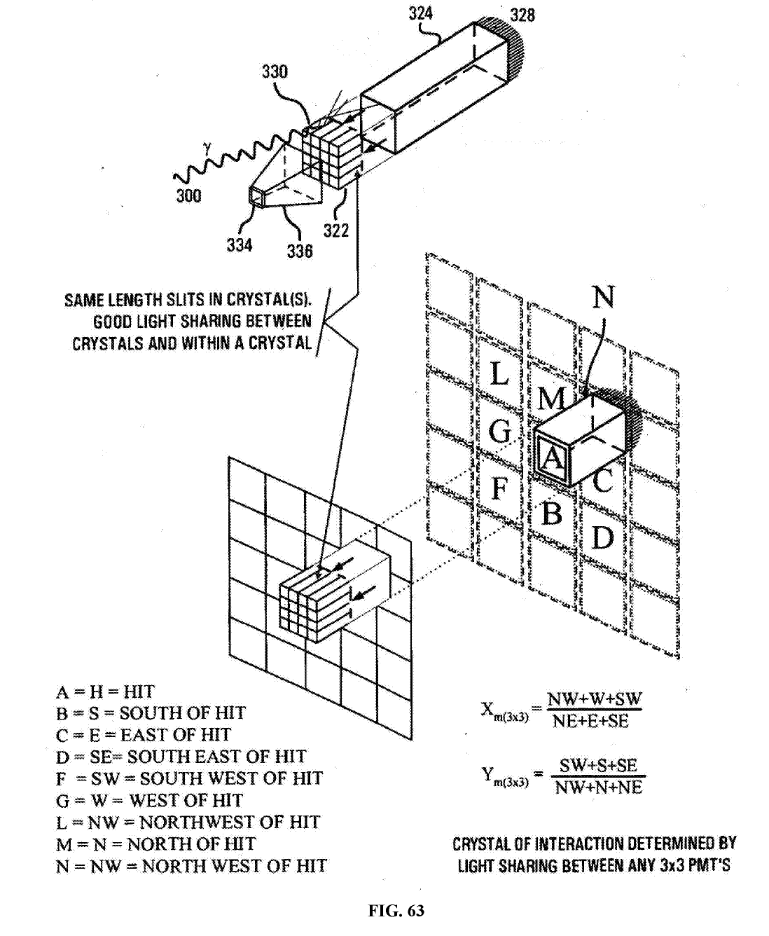

FIG. 61 shows a simple implementation of a cylindrical detector 102 made of crystals (e.g. BGO) 330 with slits (cuts between crystals) of equal length as shown in FIG. 63 with light-sharing for the portion of the crystals without slits, or made of a solid crystal 331 with light sharing with adjacent crystals as shown in FIG. 62.

The crystal can have a dimension larger than the larger sensor 324 PMT, SiPM or APD, coupled at the external surface of the cylinder. The shape of the crystal can be rectangular 6 cm×2.5 cm×20 cm, or other dimensions or trapezoidal of dimensions up to 6 cm×20 cm on the outer face, 4 cm×20 on the inner face and 2.5 cm thick, or other dimensions.

The outer sensors can be APD or PMT 1″, 1½″ or 2″, or other dimensions. Or SiPM single sensor or array of sensors (for example from Hamamatsu MPPC S13361-3050NE-04, or similar series with 4×4 number of channels, each channel area 3 mm×3 mm, or 6 mm×6 mm, or other dimensions and/or other number of channels.

The inner sensor 334 of FIG. 61, FIG. 62, FIG. 63, must be a SiPM or APD with a single sensor with a dimension smaller that the corresponding outer sensor to limit the cost. The inner sensor is coupled to the inner face of the crystal with a light-guide.

The selection of each component will be determined experimentally with measurements aimed to find the best compromise between price and efficiency, among which, efficiency has higher priority.

The function of the outer sensor 324 is to provide the best photon arrival time, energy and signals to be interpolated with signals from adjacent sensors to calculate the “x”, “y”, and “z” coordinates of the photon's impact point in the crystal.

The function of the inner sensor 334 is to provide the signal to the 3D-Flow processor to calculate the Depth of Interaction. It should be thin and not very large to minimize the obstruction of the path of the photon toward the crystal.

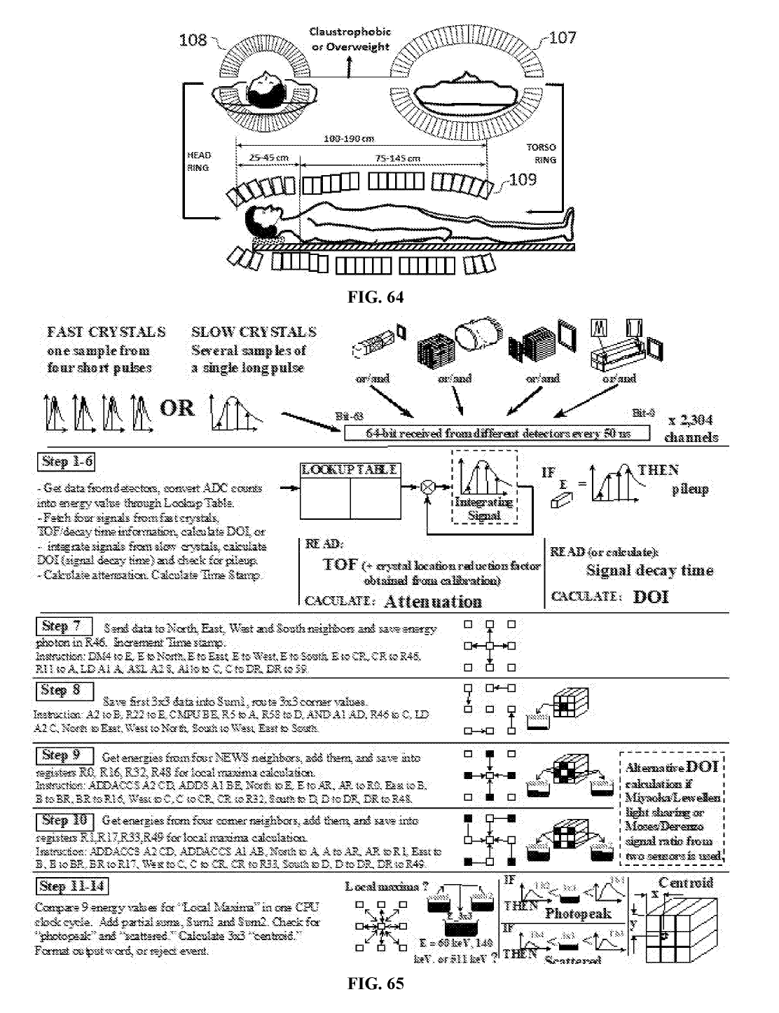

After having built, characterized and measured the efficiency and performance of at least three 3D-CBS devices with a cylindrical detector with BGO crystals, the detector shape will be modified for a smaller cylinder 108 for the head and an elliptical shape-like detector of FIG. 64 with an elliptical detector 107 for the torso of the body, having the crystals 109 at the edge of the head and at the legs, more inclined pointing toward the center of the cylinder-detector.

These modifications of the 3D-CBS detector shape are aimed to improve efficiency, by reducing the path travelled by the photons before hitting the crystal which contributes to reduce Randoms and Multiple events and also to reduce cost in a smaller volume of crystals and a smaller number of sensors.

Optimizations are obtained experimentally using a gantry that the inventor has already built that allows to move each detector block independently at a different distance from the patient's body.

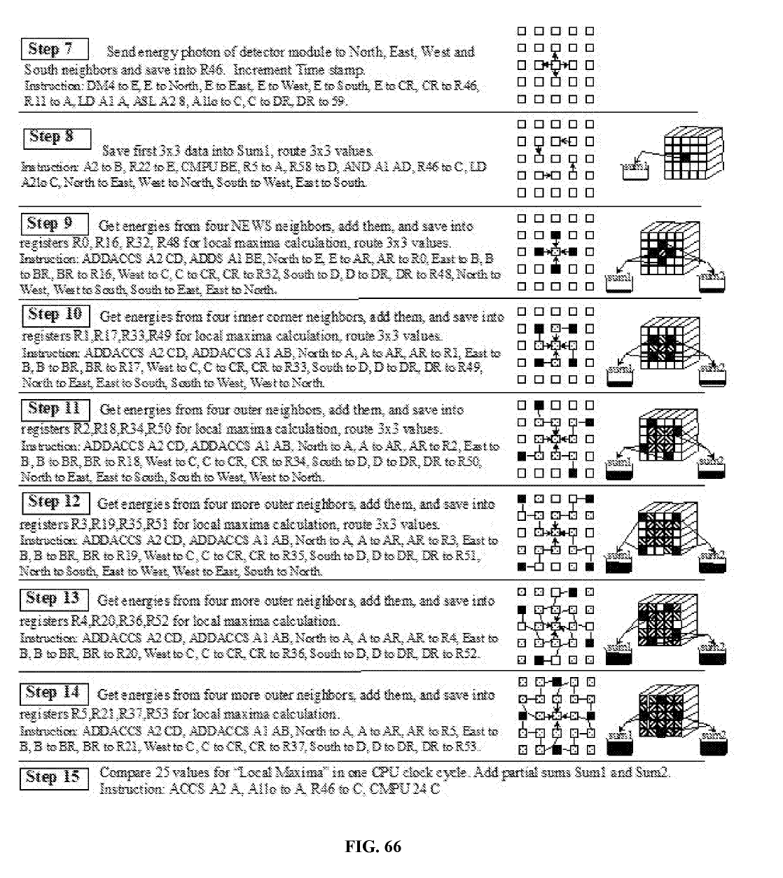

FIG. 65 illustrates some examples of real-time Object Pattern Recognition Algorithms executed by each 3D-Flow processor. The specific example is suitable for particle and photon detection and measurement on a 3×3 sensors array. FIG. 66 is describing a real-time 3D-Flow algorithm receiving and processing information from a 5×5 sensors array.

The inventive step of this invention is not limited to the current instruction set of the 3D-Flow processor capable to execute up to 26 operations in 3 nanoseconds such as add, subtract, compare with 24 values, etc. but this instruction set can be changed to more efficiently execute specific instructions needed for finding specific objects (for example the 3D-Flow processor, in addition to the normal arithmetic and logic instructions of a standard computer, has an instruction to compare 24 values in one cycle which is specific for finding a center of gravity in particle detection.

P. Timeline of Major Activities

The detailed timeline for the development of the 3D-Flow OPRA and the 3D-CBS is reported in FIG. 71. It is based on 59 quotes of different components received from industries (for each component I received two to three quotes) The phases of the work plan synchronizing the delivery of the different components assembled by a team of 12 people, estimates it will take 2 years.

At the end of the 2 years, the following units are planned to be fully tested:
The 3D-Flow ASIC with 64×3D-Flow processors
Two ER/DSU units each with 8,192 channels at 320 MHz per channel to replace the functionality of the current LHC power allowing to test the efficiency of the CMS, Atlas, 3D-Flow OPRA and other Level-1 trigger systems, as well as the 3D-CBS and other devices for medical imaging.
Two ER/DSU units each with 4,096 channels at 640 MHz per channel to replace the functionality of the future LHC at higher power and luminosity allowing to test the efficiency of the CMS, Atlas, 3D-Flow OPRA and other Level-1 Trigger systems with more data from additional sub-detectors.
One 3D-Flow unit in 16×VXI boards with 8,192 channels capable of sustaining 1.3 TB/sec input data rate for Level-1 Trigger applications in physics.
One 3D-Flow unit in 16×VME boards with 4,096 channels capable to sustain 0.65 TB/sec for Level-1 Trigger applications in physics or by using only 9×VME boards with 2,304 channels capable of sustaining 368 GB/sec for the 3D-CBS medical imaging application.
With an additional team of 4 people working with the team of 12 people planned in the timeline of FIG. 71, the parts of the detector of the 3D-CBS of FIG. 61, FIG. 62 and FIG. 63 outsourced to several companies could be assembled and three 3D-CBS units tested. The first at the end of the second year, the second at the end of the third year and the third at the end of the fourth year.

Q. Plans that Cannot Defeat Cancer

A Google search of the words “cancer breakthrough” returns 50 million hits in 0.65 seconds and 14.5 million hits in 0.74 seconds for “cancer breakthrough 2017”. (goo.gl/vmKBqC).

1. Comparison Between the 3D-CBS Technological Innovation and the Explorer PET Project with a Long Detector.

The 3D-CBS (3-D Compete Body Screening) outperforms the Explorer which was funded by NIH for $15.5 million in October 2015. However, the 3D-CBS intense computation (goo.gl/XqgnNf) capability at the front-end using 3D-Flow OPRA, provides more accurate measurements with less than 3,000 economical BGO crystals compared to 500,000 expensive LSO crystals used by the Explorer project.

The Explorer consumes 60 KW compared to 3D-CBS's 4 KW, can process only 40 TB of data in one day by a farm of computers compared to 40,000 TB per day by just 9 3D-Flow OPRA boards; plus the Explorer is ten times more expensive than the 3D-CBS.

The Explorer less efficient that the 3D-CBS, costing 25 to 35 times the current PET cannot create a paradigm change in biomedical imaging because it cannot implement a plan to save millions of lives in 30 years as described in the table of FIG. 70 and at goo.gl/4 vEEW6. There is nothing new in the Explorer, just more of everything as it was commented by several top experts in the field.

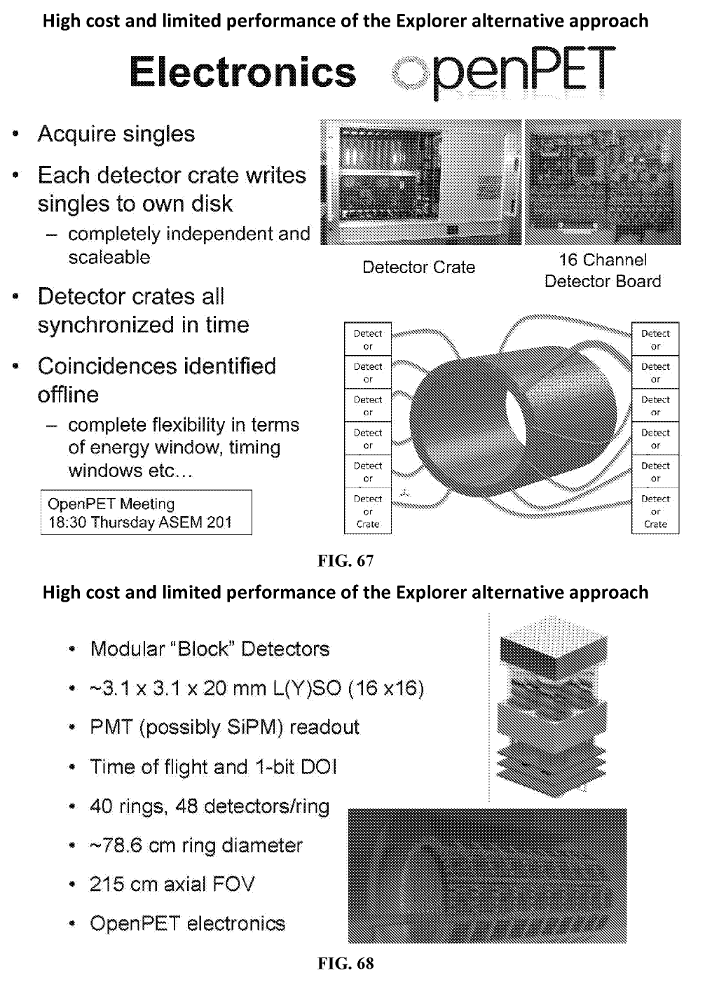

FIG. 67 is reporting the characteristics of the electronics and FIG. 68 of the detector of the Explorer project published by their authors which was also the design submitted to the National Institute of Health that granted $15.5 million for its construction.

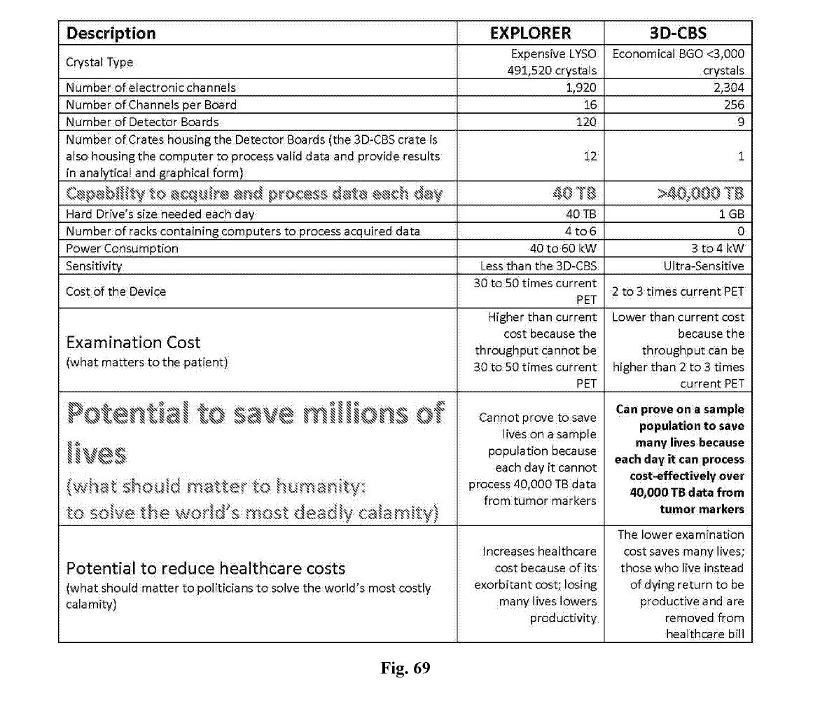

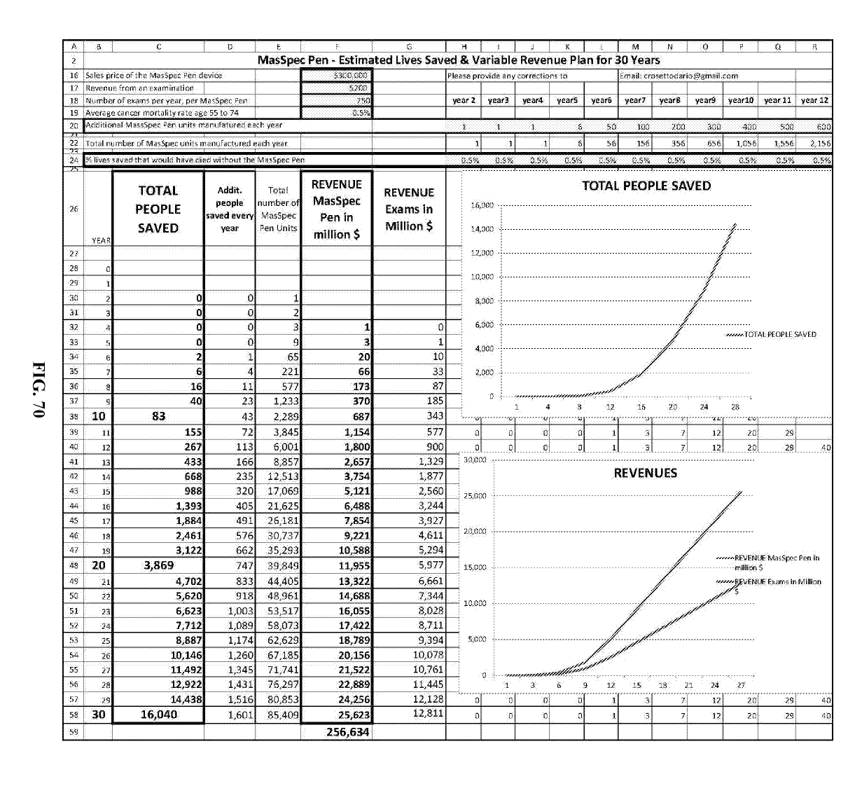

The table reported in FIG. 69 compares the features of the Explorer with the 3D-CBS features.

Data for the Explorer reported in the above table are derived from publications, slide (goo.gl/BpqjAj) presentations and several (goo.gl/RG8COf) press (goo.gl/ovMZ5j) releases (goo.gl/T195NN) made (goo.gl/NpNNNr) by the authors (goo.gl/xcBe0Q) of the (goo.gl/W6cZ9Y) Explorer and from calculations based on the data reported in the articles.

Data and feasibility (goo.gl/6DS5oy) of the 3D-CBS (goo.gl/YGg04E) (3D-Complete Body Screening) is proven by the 3D-Flow (goo.gl/5EUkYe) innovative basic concept proven feasible and functional in hardware in two modular boards (goo.gl/ymgnXz) each with 68×3D-Flow processors, and recently the 3D-Flow OPRA (goo.gl/goYPv9) was proven feasible and cost-effective by 59 quotes from reputable industries

2. The MasSpec Pen Cannot Defeat Cancer

Out of 50 million hits on cancer breakthrough in 0.65 seconds from Google search, several people asked me how to select the cancer breakthrough that would provide the highest return in premature cancer death reduction and save the most money when invested or paid through their taxes. They were particularly interested because when on Sep. 6 and 7, 2017, the MasSpec Pen (goo.gl/51C1Nd) was announced by NBC, TIME, BBC, Forbes, CBS, Today, Wired, Medscape, WebMd RAI3, and other media, I was asked which criteria could help them estimate the number of lives saved and the business revenue forecast for 30 years as I did in my table published the previous week.

Journalist should ask inventors/authors an estimate of lives and money saved by their project.

The MasSpec Pen is a useful accessory to have during surgery, however, they cannot defeat cancer.

I trust that Dr. Eberlin will provide her professional information to make a fair comparison of cost-benefits between her MasSpec Pen and other devices by updating the table of FIG. 70 with more accurate numbers as she continues testing the MasSpec Pen.

The estimate of the number of lives saved of 16,040 in 30 years, 1,601 thereafter every year assuming 85,409 total MasSpec Pen sold for a market of $85 billion in 30 years in the table may be overestimated because there are only 40,000 surgery rooms in the U.S. (See Table interactively in Excel at: goo.gl/tjBFEU, and in a static pdf document at goo.gl/vf7EXp)

R. Competitive Technologies to Save Lives

Although there have been advances in understanding and curing some specific cancers, like leukemia, results show that cures for the majority of cancers detected early work in most cases, while cures for cancers detected at a late stage usually are unsuccessful and the 50 million hits in 0.65 seconds for a Google search of “cancer breakthrough” is a lot of advertisement.

In some cases drugs for late detection, with a global cost of trillion of dollars to develop, showed initial encouraging results, but then the cancer would develop a resistance to the drug. Now many hopes are placed on Genomics, immunotherapy, CICD (Caspase Independent Cell Death), etc. These studies must be continued.

We can study the most recent cancer breakthroughs, but they either benefit only a small number of types of cancers or do not provide cost-effective advantages that would have an immediate impact that can be presented in a table similar to the table of FIG. 70.

S. The Plan that Defeats Cancer

The 3D-CBS: The First True Paradigm Change in Biomedical Imaging and the Most Competitive Technology that can Defeat Cancer

Experimental results show that early cancer detection combined with surgery, radiation therapy and chemotherapy works. Colon cancer caught early has a 91% survival rate versus 11% if caught late, breast cancer 98% vs. 27%, etc.

What is missing is a device effective for early cancer detection covering all organs of the body with a single safe examination.

This device is the 3D-CBS (3-D Complete Body Screening), an advanced PET with a 1.5 m detector covering all organs of the body (see trifold at “goo.gl/YcAJDy”, more details at “goo.gl/JMKvek” and video at goo.gl/tKGUjw) that shows competitiveness in results not only with all other technologies such as drugs for late detection, Genomic, Immunotherapy, CICD, etc., but also within all diagnostic medical imaging devices such as MRI, CT, Ultrasound, which measure tissue density and require many normal cells to change into cancerous cells before tissue morphological changes can be detected.

It is also hundreds of times more efficient and more cost effective than the existing over 10,000 PET (Positron Emission Tomography) working with the principle of detecting signals which show anomalies in biological processes before a morphological change occurs.

The 3D-CBS benefits cancer patients by ensuring all cancer cells are removed surgically, with radiation or chemotherapy and detects cancer early and effectively on asymptomatic people.

I have invented a revolutionary electronic technology-independent architecture/instrument proven feasible by 59 quotes from reputable industries to build using 2015 technology a 3D-Flow OPRA (Object Pattern real-time Recognition Algorithms) system with 8,192 channels, in 36 cm (15 inch) cube of electronics capable of sustaining over 13 TB/sec input data rate, and execute with zero dead-time on the input data, complex programmable real-time Object Pattern Recognition Algorithms (OPRA) at a production cost of approximately $100,000 (compared to the cost of over $100 million of the less efficient CMS Level-1 Trigger at CERN).

In the same volume of 36 cm cube of electronics, it is feasible to build a 3D-Flow OPRA system with 20,000 channels capable of sustaining over 20 TB/sec input data rate. Larger systems can be built by linking several similar cubes of electronics.

It overcomes the limitations of Moore's law

It can provide disruptive advantages in several applications in making computers more capable overcoming since 1992 the limits of Moore's laws that is now dead (goo.gl/2CLTsP).

Computers are made more capable by the 3D-Flow basic invention at goo.gl/NQ8Cck
1. extracting all valuable information from radiation to identify rare particles from ultra-high data rate that cannot be stored on hard drives because in one day will fill all hard drives of the planet,
2. extracting all valuable information from radiation to identify all valuable signals related to tumor markers for an effective early cancer detection requiring a very-low radiation to the patient,
3. in industrial quality control, etc.

Proven feasible by a major public scientific review and by 59 quotes from reputable industries.

My basic 3D-Flow parallel-processing architecture summarized in one page at ‘goo.gl/NQ8Cck’, recognized valuable by academia, industry and research centers at a major official, formal, international scientific review, held at Fermilab in 1993 (goo.gl/zP76Tc), proven feasible and functional in FPGA hardware, when in synergy with the implementation in its different parts using 2015 technology is summarized in two pages at ‘goo.gl/AoszvQ’ and detailed at ‘goo.gl/w3XIZ1’.

The combination of these elements provided the inventive step of the new 3D-Flow OPRA technology with unprecedented advantages. (See details about the essence of the advantages of the 3D-Flow OPRA at goo.gl/dYGusD).

My 3D-Flow invention could have replaced hundreds of crates containing 4,000 electronic boards of CERN CMS L-1 Trigger Experiment at a fraction of the cost, while providing more performance, and could have replaced the trigger at many other experiments at CERN.

For example the 3D-Flow system could have replaced the hundreds of crates containing 4,000 electronic boards of CERN CMS L-1 Trigger Experiment:
a) in 1994 with a cylinder of electronics 1.8 m tall×1 m in diameter,
b) in 1999 with 6×9U VME crates as described in the 45-page peer-reviewed article published by Nuclear Instruments and Methods in Physics Research, Sec. A, vol. 436, (1999) pp. 341-385.

Using 2015 technology can replace hundreds of crates of electronics (goo.gl/mPHw5Y) containing 4,000 electronic boards with one crate containing 9 of my 3D-Flow OPRA boards, while providing higher performance at one thousandth the cost (summarized in two pages at goo.gl/AoszvQ detailed at goo.gl/w3XIZ1).

### Results can be Measured Using the ER/DSU Unit

These claims can be proven analytically and ultimately by experimental results showing which system can find more sought particles within real LHC data at a very high luminosity in a controlled environment where it is known how many sought particles are memorized in the ER/DSU unit, will prove which system is the best.

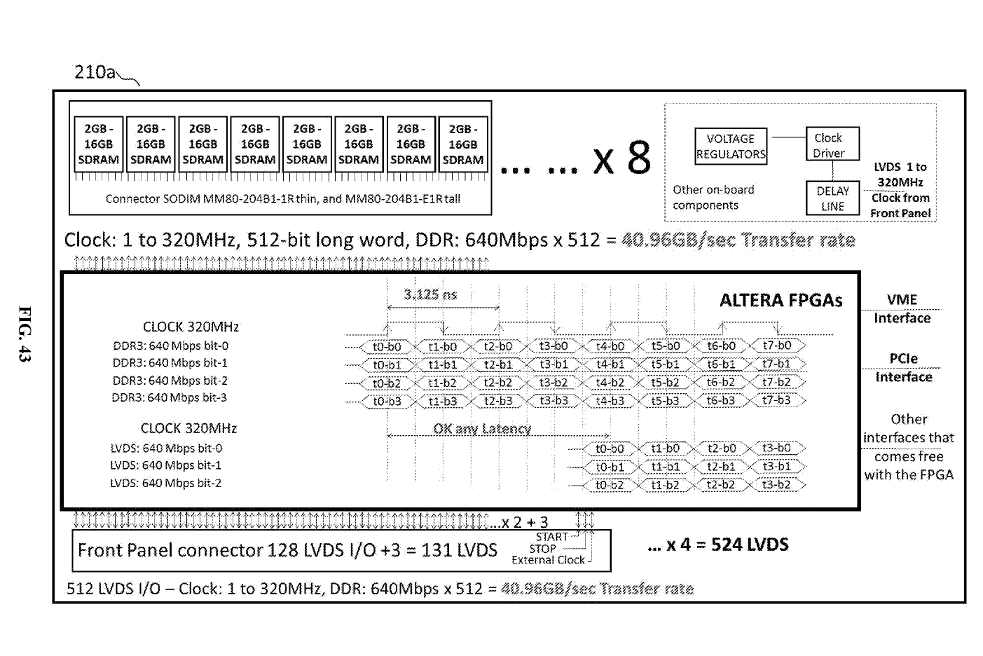

The ER/DSU unit FIG. 39, FIG. 40, FIG. 41, FIG. 42, FIG. 43, FIG. 44, FIG. 45, FIG. 46, FIG. 47 costing only $40,000 would be able to record raw data from the LHC (Large Hadron Collider) apparatus and then replay the same data to the 3D-Flow OPRA system and to the CMS Level-1 Trigger (or other trigger system) proving their enormous difference in efficiency and cost-effectiveness.

### Used in the 3D-CBS, Provides the First True Paradigm Change in Biomedical Imaging

When the 3D-Flow OPRA invention is used in the 3D-CBS (3-D Complete Body Screening) technology, it makes it hundreds of times more efficient than the over 10,000 PET (Positron Emission Tomography) devices used in hospitals providing for the first time a true paradigm change in biomedical imaging.

(See the 2000 book: “400⁺ times improved PET efficiency for lower-dose radiation, lower-cost cancer screening at ‘goo.gl/ggGGwF’, see trifold at “goo.gl/YcAJDy”, more details at “goo.gl/JMKyek”, the five-page 2003 article at ‘goo.gl/RiIn0B’, the 32-page 2013 article at “goo.gl/qpnNxd”, one-page innovations at “goo.gl/3AFCWM”, one-page benefits at ‘goo.gl/Zx1p9Q’, two-page 2016 summary and comparison with the Explorer at: ‘goo.gl/OLuA1n’ and the source information from the authors of the “Explorer Project” at ‘goo.gl/T195NN’ or at ‘goo.gl/ovMZ5j’, which was funded by NIH for $15.5 million although less efficient, incapable of saving many lives and more than ten times expensive as the 3D-CBS).

The purpose of this proposal/invention/project is to create a general purpose 3D-Flow OPRA (Object Pattern Real-Time Algorithm) instrumentation that can identify objects in real-time, display their characteristics, and provide flexible triggering features using fast neighboring data exchange and by analyzing data for a time longer than the time interval between two consecutive input data which arrive in parallel from a matrix of thousands of transducers at ultra-high speed.

The 3D-Flow OPRA instrument can also solve multiple applications in different fields where the requirements are to identify among millions of other non-relevant signals cluttering the good information, the signals related to specific objects such as pairs of 511 keV photons, the profile and details of a face among thousands of faces, the signals characterizing the Higgs boson particle, etc.

By allowing the user to define a complex real-time algorithm which analyzes signals arriving from a matrix of transducers that respond to physical stimuli it allows to measure all kinds of phenomena and identify all kinds of objects in 3-D.

The 3D-Flow OPRA instrument for example can recognize a shape of different colors, a shape of different levels of heat, a shape of different levels of sound volume and frequencies, a shape of different energies, a shape of different mechanical stress, a shape of different pressure, a shape of different light, the characteristics of a specific subatomic particle measured from signals generated by CCD, APD, PMT, SiPM, PADs, silicon strip detectors, wire-chambers, drift-chambers, etc.

In addition, this proposal/project is also providing the method and apparatus to build an instrument, the ER/DSU (Event Recorder and Detector Simulator Unit) with the capability to record in a memory, raw data, and replay them at the real-time speed to the 3D-Flow OPRA or 3D-CBS systems under test to measure their performance in finding unknown objects.

3. Protecting the Investment and Saving Lives

My patents are protecting investors who care to make a difference in the world. My basic inventions have been proven feasible and functional in hardware. My latest improvements give an additional advantage in lower cost and higher efficiency compared to my original design of the 3D-CBS from the year 2000.

My patented invention has the capability to accurately capture all possible signals from the tumor markers at the lowest cost per valid signal captured compared to alternative approaches.

Fifty-nine quotes from reputable industries prove enormous advantages of the 3D-CBS compared to the most advance PET with a long 2 m detector, the Explorer.

The differences are detailed in a comparison table of FIG. 69 with references to documents written by the respective authors.

My patent protects the investor with a large margin in competitivity which goes from the component costs to build my 3D-CBS to the component cost for the competitors to build their Explorer.

For example, the cost of the components for the 3D-CBS is less than $2 million, while for the Explorer cost is more than $20 million.

In the table of FIG. 72 I selected a selling price for the 3D-CBS of $3.5 million, which gives a reasonable profit to the investor; however, for a quicker return of the investment, the selling price can be set anywhere below the Explorer's price which they would have to be set at over $20 million—their component cost—if they want to make a profit. The same criteria can be used for the examination cost that I selected as $400 but could be increased to over $1000 and still remain competitive.

These are good incentives to start a new market with the 3D-CBS to save lives and later the competition will lower the price of the goods.

A diligent work in determining the other values in the yellow cells of the spreadsheet will provide an estimate of revenues and lives saved.

Three 3D-CBS units located in different countries (USA4, Canada, Italy) will constantly measure performance to confirm or modify the estimates in this table as follows: each unit screening 10,000 people in the age group 55 to 74 taken from a location where, in the previous 20 years, the mortality rate was constant (e.g. 0.5%) and reporting every year changes in the death rate.

See interactive Table in Excel at: goo.gl/BnrUdc and in pdf at: goo.gl/mgCJFq

There are no technical or economic reasons preventing the defeat of cancer, saving 16 million lives in 30 years and 50% thereafter.

Let's be united against cancer, cooperate to enforce the rule of law and transparency in science which will remove roadblocks to receive benefits in a significant premature cancer deaths reduction that could already have been put into action 17 years ago.

### THE INVENTIVE STEPS THAT CAN REPLACE 4,000 ELECTRONIC BOARDS OF THE CMS L-1 TRIGGER AT CERN WITH 9 OF THIS 3D-FLOW OPRA BOARD WHICH PROVIDE ENORMOUS PERFORMANCE IMPROVEMENT AT ONE THOUSANDTH THE COST AND THAT ALLOWS TO IMPLEMENT A 30 YEARS PLAN TO SAVE OVER 16 MILLION PEOPLE FROM DYING PREMATURELY FROM CANCER

The inventive steps of the method and apparatus of this invention is the synergy and combinations of several inventions I made in the past with new ones which together allow to achieve enormous performance increase in several fields of applications. Described herein are for high energy physics experiment applications and for medical imaging.

Following are listed a few of the elements that when placed together in a certain way as described in this invention provide global advantages and benefit in many fields.

All components concur to keep short distances which is important in executing pattern recognition algorithm were there is the need to exchange data between neighboring elements, processors, etc. The latency to exchange data with neighbors should be as short as possible in time. The transfer of 8,192-bit word where each bit should arrive at the same time is more cost-effective to transfer them over 8,192 cables at 1 MHz rather than through 1/100 cables at 100 times the speed but with the need of transmitters/receivers and an overhead time by SERTA to serialize and de-serialize with error recovery. The economical BGO crystal available in abundance in the market is a better plan than using the more expensive LSO which needs an element (lu), which is rare in nature. The multiplexing of the signals to the adjacent processors (North, East, West, South ports of the 3D-Flow processors) have been optimized for best compromise in number of I/O, cost of the package, speed, power consumption, etc.

Each component in this method and apparatus has been chosen and will continue to be chosen and optimize for the best results from the table at FIG. 72 that should maximize reduction of cancer death in a 30 years period and beyond. For high energy physics the choice of the components has been selected to give the most powerful instrument to experimental physicists to discover the unknown.

### SEQUENCE LISTING (IF ANY)

No sequential list is provided for this application

What is claimed is:

1. A technology-independent, modular, scalable, ultra-high performance, low cost 3D-Flow OPRA system, implementable in different hardware form factors such as VXI, VME, VPX, ATCA, etc. for processing data signals arriving from a matrix of transducers or from an array of memory modules at several Terabytes per second per each crate, executing programmable complex Object Pattern Recognition Algorithms (OPRA) in real-time and capable of executing several functions when the desired trigger condition on the object sought is met. The innovative steps of this invention provide disruptive advantages compared to the state of the art. A 3D-Flow OPRA system for approximately 10,000 channels, sustaining an input data rated of a few Terabyte per second can be built in a VXI crate at a component and manufacturing cost of the order of $100,000. In comparison, the state of the art at CERN Research Laboratory in Geneva built a system with fewer number of channels, less performant at a cost of over one hundred million dollars.

2. The system of claim 1 comprising:
(a) A 3D-Flow OPRA processor with the capability of bidirectional communication in four directions and unidirectional communication in additional two directions. With the capability to execute uninterruptable algorithm for a time longer than the time interval between two consecutive input data
(b) A set of different electronic boards with different features in terms of number of inputs, the complexity of the OPRA algorithms can execute in real-time, the maximum speed of the input data rate it can sustain and the features provided when the desired trigger (or matching) condition is met.
(c) A set of different features such as saving data signals when the condition is met, saving data signals before and after the condition is met, taking an immediate action when the condition is met (such as in a closed-loop controlled system)

3. The system of claim 1 implemented in VXI 9U boards

4. The system of claim 1 implemented in VME 6U boards

5. A technology-independent, modular, scalable, high-speed, low cost data recorder and generator ER/DSU (Event Recorder and Detector Simulator Unit) implemented in several hardware form factors capable of reaching a transfer rate in/out of the order of a few Terabyte per second on a 9U VXI crate. A ER/DSU unit for approximately 10,000 channels, sustaining an input data rated of a few Terabyte per second can be built in a VXI crate at a component and manufacturing cost for less than $100,000. Commercially available instrumentation with less performance cost over ten times.
